(19) United States
(12) Patent Application Publication
(10) Pub. No.: US 2023/0271770 A1
(43) Pub. Date: Aug. 31, 2023

(54) Anti-Viral Pharmaceutical Formulations Administered Via Devices for Lung Targeted Delivery

(71) Applicant: Amphastar Pharmaceuticals, Inc., Rancho Cucamonga, CA (US)

(72) Inventors: Jack Yongfeng Zhang, Rancho Cucamonga, CA (US); Mary Zi-ping Luo, Rancho Cucamonga, CA (US); Ying Wang, Rancho Cucamonga, CA (US); Yi Xia, Rancho Cucamonga, CA (US); Jie Fei Ding, Rancho Cucamonga, CA (US); Selina Su, Rancho Cucamonga, CA (US); Qingxia Han, Rancho Cucamonga, CA (US)

(21) Appl. No.: 17/910,405

(22) PCT Filed: Apr. 21, 2021
(86) PCT No.: PCT/US2021/028490

§ 371 Date: Sep. 9, 2022

Related U.S. Application Data
(60) Provisional application No. 63/019,997, filed on May 4, 2020.
(60) Provisional application No. 63/019,981, filed on May 4, 2020.
(60) Provisional application No. 63/019,978, filed on May 4, 2020.
(60) Provisional application No. 63/019,974, filed on May 4, 2020.
(60) Provisional application No. 63/013,405, filed on Apr. 21, 2020.

(51) Int. Cl. B65D 83/54; A61M 15/00; B65D 83/38; B65D 83/30; B65D 83/14; B65D 83/36

(52) U.S. Cl. CPC B65D 83/54; A61M 15/009; B65D 83/386; B65D 83/303; B65D 83/7532; B65D 83/36; B65D 2583/005

(57) ABSTRACT

The present disclosure generally pertains to a medical device and more particularly, a metered-dose inhaler (“MDI”) actuator capable of a targeted delivery of fine API particles having particle diameters of about 1.1 μm or less to a portion of a patient's lungs where alveoli are located.

### CROSS-REFERENCE TO RELATED APPLICATIONS

The present application claims the benefit of U.S. Provisional Application Nos. 63/013,405, filed on Apr. 21, 2020; 63/019,974, filed on May 4, 2020; 63/019,978, filed on May 4, 2020; 63/019,997, filed on May 4, 2020; and 63/019,981, filed on May 4, 2020, all of which are incorporated by reference herein in their entireties.

### FIELD

Embodiments described herein generally relate to pharmaceutical formulations delivered by metered-dose inhalers (“MDI's”) to the respiratory tract, including the deep lung, and methods for targeted delivery of pharmaceutical formulations for antiviral treatment. Specifically, embodiments described herein relate to pharmaceutical formulations and MDI's capable of delivering size-controlled HCQ particles to a portion of a patient's lungs where alveoli are located to treat a pulmonary disease such as COVID-19. Embodiments described herein show the safety, effectiveness, the absorption, and pharmacokinetics of HCQ, which are demonstrated by analyzing the HCQ concentrations in lungs of mice.

### BACKGROUND

COVID-19 is an infectious disease caused by a virus known as SARS-CoV-2 (hereinafter, referred to as “CoV2”) CoV2 can infect and damage multiple human organs; however, the damage CoV2 can cause to the lungs is often the most critical and detrimental. CoV2 typically enters the human body through the nose and/or mouth, then travels along the airway tract into the lungs. Once in the alveoli, CoV2 uses its distinctive spike-shaped proteins to “hijack” cells. When CoV2's RNA has entered a hijacked cell, new copies of CoV2 are made. This replication process kills the hijacked cells, which allows for the new copies of CoV2 to be released out of the hijacked cell to infect neighboring cells in the alveolus. CoV2's process of hijacking cells to reproduce causes inflammation in the lungs, which triggers an immune response. As this process unfolds, fluid begins to accumulate in the alveoli, causing a dry cough and making breathing difficult. This process can also cause severe alveolar damage, which is a major cause of morbidity and mortality in affected COVID-19 patients.

Both hydroxychloroquine (“HCQ”) and chloroquine (“CQ”) oral tablets have been used as an off-label oral treatment for combating CoV2. However, the effectiveness of HCQ oral tablets in treating COVID-19 has not been proven, and the tablets may have significant efficacy and safety limitations. For example, high doses of HCQ can result in serious cardiovascular complications. Further, only a low concentration, contributed by 0.07% of HCQ oral tablet dose, is distributed to the plasma, and ends up in alveoli. As a result, with this extremely low concentration via an HCQ oral dose, it may be ineffective in fighting against CoV2. Consequently, this results in insufficient efficacy in treating CoV2, and other pulmonary viral diseases. Furthermore, the oral dose delivery distributes the drug systemically, i.e., throughout the body, and spreads thin. As a result, the drug particle cannot reach the effective concentration in the infected alveolar cells within the lungs to combat CoV2.

Accordingly, there is a need for a method to safely administer HCQ to a patient in a manner that targets the alveoli. By delivering the drug directly to the alveoli, a lower dose of HCQ may be sufficient to be administered while drastically increasing the efficacy of the drug within the lung tissue that has been infected by CoV2 in order to treat the disease.

### BRIEF SUMMARY

The present disclosure is directed to targeted delivery of HCQ pharmaceutical formulations for antiviral treatment within the respiratory tract, including the deep lung area. The targeted delivery may be achieved via MDI actuators, which may be configure for stand-alone use, such as handheld, self-administrable actuators, or may be configured for use with an auxiliary delivery component, for example a ventilator.

Some embodiments are directed to a metered-dose inhaler (“MDI”) actuator for self-administration of pharmaceutical formulations. The MDI actuator may be a handheld actuator for dispensing, via actuation, a pharmaceutical formulation from an MDI into a patient, the pharmaceutical formulation having at least one active pharmaceutical ingredient (API), where the MDI is capable of administering a portion of the at least one API to a portion of a lung where a plurality of alveoli are located, and where the MDI actuator includes a nozzle having an inner diameter of 0.15 mm to 0.3 mm.

In some embodiments, an inner diameter of the nozzle according to the previous embodiment is about 0.18-0.25 mm. In some embodiments, the inner diameter of the nozzle is about 0.20-0.23 mm.

In some embodiments, the portion of the lung where the plurality of alveoli are located according to either of the previous two embodiments includes at least Stage 6 based on a Cascade Impactor particle size distribution of a respiratory tract, where Stage 6 has a particle diameter size of about 1.1 μm or less.

In some embodiments, the MDI actuator according to any of the previous embodiments is capable of providing a delivery efficiency rate of at least 25.0%, where the delivery efficiency rate is determined by dividing (i) a total amount, per actuation, of an API having a particle diameter of less than 1.1 μm, by (ii) an expected API metered dose per actuation.

In some embodiments, the MDI actuator according to any of the previous embodiments is capable of providing a delivery efficiency rate of at least 25.0%, where the delivery efficiency rate is determined by dividing (i) a total amount, per actuation, of an API having a particle diameter of less than 1.1 μm, by (ii) an expected API metered dose per actuation, where the API is hydroxychloroquine (HCQ), and the API dose strength per actuation is 400 μg.

In some embodiments, the pharmaceutical formulation according to any of the previous embodiments includes a pharmaceutical formulation suitable for inhalation.

In some embodiments, the pharmaceutical formulation according to any of the previous embodiments includes a pharmaceutical formulation suitable for inhalation, and further includes an API including an anti-viral therapeutic agent, where the anti-viral therapeutic agent includes HCQ, a free base thereof, or a pharmaceutically acceptable salt thereof.

In some embodiments, the pharmaceutical formulation according to any of the previous embodiments is indicated for the treatment of a pulmonary disease.

In some embodiments, the pharmaceutical formulation according to any of the previous embodiments is indicated for the treatment or prophylaxis of COVID-19.

In some embodiments, the patient according to any of the previous embodiments has one or more pulmonary diseases. In some embodiments, the patient has one or more pulmonary diseases, including at least COVID-19.

In some embodiments, the MDI according to any of the previous embodiments includes a container, where the container is a pressurized canister for dispensing, per actuation, a metered dose of the pharmaceutical formulation.

In some embodiments, the nozzle according to any of the previous embodiments has a jet length of 0.5 mm to 1.0 mm. In some embodiments, the nozzle has a jet length of about 0.7 mm.

In some embodiments, the pharmaceutical formulation according to any of the previous embodiments further includes: an alcohol of about 5% (w/w) of the pharmaceutical formulation; and a propellant of about 95% (w/w) of the pharmaceutical formulation, where “w/w” denotes weight by weight.

In some embodiments, the pharmaceutical formulation according to any of the previous embodiments further includes: an alcohol of about 5% (w/w) of the pharmaceutical formulation, where the alcohol is ethanol alcohol (“EtOH”); a propellant of about 94.6% (w/w) of the pharmaceutical formulation, where the propellant is HFA-134a, where the HCQ is about 0.4% (w/w) of the pharmaceutical formulation, where the HCQ is free base, where the pharmaceutical formulation is a true solution, where the pharmaceutical formulation has a total weight of about 8-12.5 grams, and where “w/w” denotes weight by weight.

In some embodiments, the pharmaceutical formulation according to any of the previous embodiments includes an inhalable steroid.

In some embodiments, the inhalable steroid according to the previous embodiment is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

In some embodiments, the pharmaceutical formulation according to any of the previous embodiments includes a bronchodilator.

In some embodiments, the bronchodilator according to the previous embodiment is selected from the group consisting of albuterol, levosalbutamol, pirbuterol, epinephrine, racemic epinephrine, ephedrine, terbutaline, salmeterol, formoterol, bambuterol, indacaterol and pharmaceutically acceptable salts thereof.

In some embodiments, the pulmonary disease according to any of the previous embodiments is selected from the group consisting of asthma, chronic obstructive pulmonary disease (COPD), sarcoidosis, eosinophilic pneumonia, pneumonia, interstitial lung disease, bronchiolitis, bronchiectasis, and restrictive lung diseases.

Some embodiments are directed to a method for self-administration of a pharmaceutical formulation, the method including: dispensing, via actuation, using a self-administrable, handheld MDI actuator, a pharmaceutical formulation from a MDI into a patient, the pharmaceutical formulation having at least one API, where the MDI is capable of administering a portion of the at least one API to a portion of a lung where a plurality of alveoli are located, and where the MDI actuator includes an nozzle having an inner diameter of 0.15 mm to 0.3 mm.

In some embodiments, the inner diameter of the nozzle according to the previous embodiment is about 0.18-0.25 mm. In some embodiments, the inner diameter of the nozzle is about 0.20-0.23 mm.

In some embodiments, the portion of the lung where the plurality of alveoli are located according to either of the previous two embodiments includes at least Stage 6 based on a Cascade Impactor particle size distribution of a respiratory tract, where Stage 6 has a particle diameter size of about 1.1 μm or less.

In some embodiments, the MDI actuator according to any of the previous three embodiments is capable of providing a delivery efficiency rate of at least 25.0%, where the delivery efficiency rate is determined by dividing (i) a total amount, per actuation, of an API having a particle diameter of less than 1.1 μm, by (ii) an expected API metered dose per actuation.

In some embodiments, the MDI actuator according to any of the previous four embodiments is capable of providing a delivery efficiency rate of at least 25.0%, where the delivery efficiency rate is determined by dividing (i) a total amount, per actuation, of an API having a particle diameter of less than 1.1 μm, by (ii) an expected API metered dose per actuation, where the API is HCQ from an HCQ inhalation pharmaceutical formulation, and the API dose strength per actuation is 400 μg.

In some embodiments, the pharmaceutical formulation according to any of the previous five embodiments includes a pharmaceutical formulation suitable for inhalation.

In some embodiments, the pharmaceutical formulation according to any of the previous six embodiments includes a pharmaceutical formulation suitable for inhalation, and further includes an API comprising an anti-viral therapeutic agent, where the anti-viral therapeutic agent comprises HCQ, a free base thereof, or a pharmaceutically acceptable salt thereof.

In some embodiments, the pharmaceutical formulation according to any of the previous seven embodiments is indicated for the treatment of a pulmonary disease.

In some embodiments, the pharmaceutical formulation according to any of the previous eight embodiments is indicated for the treatment or prophylaxis of COVID-19.

In some embodiments, the patient according to any of the previous nine embodiments has one or more pulmonary diseases. In some embodiments, the patient has one or more pulmonary diseases, including at least COVID-19.

In some embodiments, the MDI according to any of the previous ten embodiments includes a container, where the container is a pressurized canister for dispensing, per actuation, a metered dose of the pharmaceutical formulation.

In some embodiments, the nozzle according to any of the previous eleven embodiments has a jet length of 0.5 mm to 1.0 mm. In some embodiments, the nozzle has a jet length of about 0.7 mm.

In some embodiments, the pharmaceutical formulation according to any of the previous twelve embodiments further includes an alcohol of about 5% (w/w) of the pharmaceutical formulation; and a propellant of about 95% (w/w) of the pharmaceutical formulation, where “w/w” denotes weight by weight.

In some embodiments, the pharmaceutical formulation according to any of the previous thirteen embodiments further includes: an alcohol of about 5% (w/w) of the pharmaceutical formulation, where the alcohol is ethanol alcohol (“EtOH”); a propellant of about 94.6% (w/w) of the pharmaceutical formulation, where the propellant is HFA-134a, where the HCQ is about 0.4% (w/w) of the pharmaceutical formulation, where the HCQ is free base, where the pharmaceutical formulation is a true solution, where the pharmaceutical formulation has a total weight of about 8-12.5 grams, and where “w/w” denotes weight by weight.

In some embodiments, the pharmaceutical formulation according to any of the previous fourteen embodiments includes an inhalable steroid.

In some embodiments, the inhalable steroid according to the previous embodiment is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

In some embodiments, the pharmaceutical formulation according to any of the previous sixteen embodiments includes a bronchodilator.

In some embodiments, the bronchodilator according to the previous embodiment is selected from the group consisting of albuterol, levosalbutamol, pirbuterol, epinephrine, racemic epinephrine, ephedrine, terbutaline, salmeterol, formoterol, bambuterol, indacaterol and pharmaceutically acceptable salts thereof.

In some embodiments, the pulmonary disease according to any of the previous embodiments is selected from the group consisting of asthma, chronic obstructive pulmonary disease (COPD), sarcoidosis, eosinophilic pneumonia, pneumonia, interstitial lung disease, bronchiolitis, bronchiectasis, and restrictive lung diseases.

Some embodiments are directed to an MDI actuator for ventilator-delivery of pharmaceutical formulations. The MDI actuator may be configured for dispensing, via actuation, a pharmaceutical formulation from an MDI into a ventilator connector, where the ventilator connector is capable of operatively connecting to a patient and a ventilator via ventilator circuitry. The MDI may include a container having the pharmaceutical formulation, and may be capable of dispensing a metered dose, per actuation, of the pharmaceutical formulation. The MDI actuator may include an insert having: a length of 10.0 mm to 20.0 mm, an inner diameter of 0.5 mm to 2.5 mm; an outer diameter of 4.0 mm to 5.0 mm; and a nozzle having an inner diameter of 0.15 mm to 0.25 mm and a jet length of 0.5 mm to 1.0 mm; a tapered stem block having an inner diameter of 2.5 mm to 3.5 mm towards its distal end and tapered outward towards its proximal end. The MDI actuator may be configured to produce a sump volume of 5.0 μL to 45.0 μL, and may include a body for aligning the MDI for dispense by the MDI actuator and a connector fitting for connecting to a corresponding connector fitting of the ventilator connector.

In some embodiments, the connector fitting according to the previous embodiment is a Luer-lock fitting for connecting to a corresponding Luer-lock fitting of the ventilator connector.

In some embodiments, the pharmaceutical formulation according to either of the previous two embodiments is a pharmaceutical formulation suitable for inhalation.

In some embodiments, the pharmaceutical formulation according to any of the previous three embodiments is a pharmaceutical formulation suitable for inhalation, and further includes an API comprising HCQ, chloroquine (“CQ”), epinephrine, beclomethasone, albuterol, ipratropium, a free base thereof, a pharmaceutically acceptable salt thereof, or any combination thereof.

In some embodiments, the pharmaceutical formulation according to any of the previous four embodiments is a pharmaceutical formulation suitable for inhalation, and further includes an API comprising an anti-viral therapeutic agent, wherein the anti-viral therapeutic agent comprises hydroxychloroquine (“HCQ”), a free base thereof, or a pharmaceutically acceptable salt thereof.

In some embodiments, the pharmaceutical formulation according to any of the previous five embodiments is indicated for the treatment of a pulmonary disease.

In some embodiments, the pharmaceutical formulation according to any of the previous six embodiments is indicated for the treatment or prophylaxis of COVID-19.

In some embodiments, the patient according to any of the previous seven embodiments has one or more pulmonary diseases. In some embodiments, the patient has one or more pulmonary diseases, including at least COVID-19.

In some embodiments, the pharmaceutical formulation according to any of the previous eight embodiments includes an inhalable steroid.

In some embodiments, the inhalable steroid according to the previous embodiment is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

In some embodiments, the pharmaceutical formulation according to any of the previous ten embodiments includes a bronchodilator.

In some embodiments, the bronchodilator according to the previous embodiment is selected from the group consisting of albuterol, levosalbutamol, pirbuterol, epinephrine, racemic epinephrine, ephedrine, terbutaline, salmeterol, formoterol, bambuterol, indacaterol and pharmaceutically acceptable salts thereof.

In some embodiments, the pulmonary disease according to any of the previous twelve embodiments is selected from the group consisting of asthma, chronic obstructive pulmonary disease (COPD), sarcoidosis, eosinophilic pneumonia, pneumonia, interstitial lung disease, bronchiolitis, bronchiectasis, and restrictive lung diseases.

In some embodiments, the container according to any of the previous thirteen embodiments is a pressurized canister.

In some embodiments, the inner diameter of the nozzle according to any of the previous fourteen embodiments is 0.20 mm to 0.25 mm. In some embodiments, the inner diameter of the nozzle is about 0.20 mm. In some embodiments, the inner diameter of the nozzle is about 0.22 mm.

In some embodiments, the jet length of the nozzle according to any of the previous fifteen embodiments is about 0.7 mm. In some embodiments, the jet length of the nozzle is 11.0 mm to 21.0 mm.

In some embodiments, the length of the insert according to any of the previous sixteen embodiments is about 12 mm. In some embodiments, the length of the insert is about 15 mm. In some embodiments, the length of the insert is about 17 mm. In some embodiments, the length of the insert is about 20 mm.

In some embodiments, the inner diameter of the tapered stem block according to any of the previous seventeen embodiments is 3.1 mm to 3.5 mm towards its distal end. In some embodiments, the inner diameter of the tapered stem block is about 3.16 mm towards its distal end. In some embodiments, the inner diameter of the tapered stem block is about 2.78 mm towards its distal end.

In some embodiments, the sump volume according to any of the previous eighteen embodiments is 8.0 μL to 30.0 μL. In some embodiments, the sump volume is about 9.6 μL, about 10.3 μL, about 11.9 μL, about 12.7 μL, about 25 μL, or about 40.7 μL.

In some embodiments, the inner diameter of the insert according to any of the previous nineteen embodiments is 1.0 mm to 2.0 mm. In some embodiments, the inner diameter of the insert is about 1.0 mm. In some embodiments, the inner diameter of the insert is about 2.0 mm.

In some embodiments, the outer diameter of the insert according to any of the previous twenty embodiments is 4.0 mm to 5.0 mm, and is tapered at a slope of about 3.44° inward towards its distal end. In some embodiments, the outer diameter of the insert is about 4.4 mm.

In some embodiments, the MDI actuator according to any of the previous twenty-one embodiments further includes at least one handle support, where the at least one handle support is for engaging with at least one finger of an individual to cooperatively actuate the pharmaceutical formulation from the container.

In some embodiments, the MDI actuator according to any of the previous twenty-two embodiments further includes at least two handle supports, where the at least two handle supports are for engaging with at least two fingers of an individual to cooperatively actuate the pharmaceutical formulation from the container.

In some embodiments, the MDI actuator according to any of the previous twenty-three embodiments is made of at least one of polypropylene, polycarbonate, or acrylonitrile butadiene styrene (“ABS”).

In some embodiments, the insert according to any of the previous twenty-four embodiments includes a crown having a configuration of (i) flat, (ii) ϕ1.6 plus 90° cone, (iii) ϕ1 plus 90° cone plus ϕ3, (iv) ϕ2.78 sphere, or (v) ϕ3.18 sphere.

In some embodiments, the insert according to any of the previous twenty-five embodiments further includes a crown having a depth of 0.5 mm to 3.0 mm. In some embodiments, the crown has a depth of about 0.5 mm. In some embodiments, the crown has a depth of about 1.5 mm.

In some embodiments, the ventilator connector according to any of the previous twenty-six embodiments is ventilator tubing.

In some embodiments, the MDI actuator according to any of the previous twenty-seven embodiments is capable of providing a delivery efficiency rate of at least 25.0%, where the delivery efficiency rate is determined by dividing (i) a total amount, per actuation, of an API having a certain particle diameter, by (ii) an expected API metered dose per actuation.

In some embodiments, the MDI actuator according to any of the previous twenty-eight embodiments is capable of providing a delivery efficiency rate of at least 35.0%, where the delivery efficiency rate is determined by dividing (i) a total amount, per actuation, of an API having a certain particle diameter, by (ii) an expected API metered dose per actuation.

In some embodiments, the MDI actuator according to any of the previous twenty-nine embodiments is capable of providing a delivery efficiency rate of at least 35.0%, where the delivery efficiency rate is determined by dividing (i) a total amount, per actuation, of an API having a particle diameter of less than 1.1 μm, by (ii) an expected API metered dose per actuation, wherein the API is HCQ from an HCQ inhalation pharmaceutical formulation, and the API dose strength per actuation is 400 μg.

In some embodiments, the MDI actuator according to any of the previous thirty embodiments is made as a one-piece assembly.

In some embodiments, the body according to any of the previous thirty-one embodiments further includes one or more ribs to accommodate the container.

In some embodiments, the ventilator connector according to any of the previous thirty-two embodiments has an elbow configuration.

In some embodiments, the ventilator connector according to any of the previous thirty-three embodiments has an elbow configuration, and does not include an inner channel in proximity to its connector fitting.

In some embodiments, the ventilator connector according to any of the previous thirty-four embodiments has an elbow configuration, does not include an inner channel in proximity to the connector fitting, and the connector fitting is a Luer-lock fitting.

In some embodiments, the pharmaceutical formulation according to any of the previous thirty-five embodiments further includes: an alcohol of about 5% (w/w) of the pharmaceutical formulation; a propellant of about 95% (w/w) of the pharmaceutical formulation, where “w/w” denotes weight by weight.

In some embodiments, the pharmaceutical formulation according to any of the previous thirty-six embodiments further includes: an alcohol of about 5% (w/w) of the pharmaceutical formulation, where the alcohol is ethanol alcohol (“EtOH”); a propellant of about 94.6% (w/w) of the pharmaceutical formulation, where the propellant is HFA-134a, the HCQ is about 0.4% (w/w) of the pharmaceutical formulation, the HCQ is free base, the pharmaceutical formulation is a true solution, and the pharmaceutical formulation has a total weight of about 11.7 grams, where “w/w” denotes weight by weight.

Some embodiments are directed to a method for ventilator-delivery of a pharmaceutical formulation to a patient operatively connected to a ventilator, the method including: connecting a connector fitting on a MDI actuator to a corresponding connector fitting of a ventilator connector operatively connected to a patient and a ventilator; dispensing, via actuation using the MDI actuator, a pharmaceutical formulation from a MDI and into the ventilator connector; wherein the pharmaceutical formulation has an API, where the dispense is capable of providing a delivery efficiency rate of at least 25.0%, where the delivery efficiency rate is determined by dividing (i) a total amount, per actuation, of an API having a certain particle diameter, by (ii) an expected API dose per actuation, and where the API having the certain particle diameter is able to reach a portion of a lung where a plurality of alveoli are located.

In some embodiments, the API having the certain particle diameter according to the previous embodiment has a particle diameter of less than about 1.1 μm.

In some embodiments, the portion of the lung where the plurality of alveoli are located according to either of the previous two embodiments includes at least Stage 6 based on a Cascade Impactor particle diameter distribution of a respiratory track, where Stage 6 has a particle diameter size of about 1.1 μm or less.

In some embodiments, the portion of the lung where the plurality of alveoli are located according to any of the previous three embodiments includes at least Stage 6 and Stage 7 based on a Cascade Impactor particle diameter distribution of a respiratory track, where Stage 6 and Stage 7 include a particle diameter size in a range of 0.4 μm to 1.1 μm.

In some embodiments, the delivery efficiency rate according to any of the previous four embodiments is at least 35.0%.

In some embodiments, the connector fitting of the MDI actuator according to any of the previous five embodiments is a Luer-lock fitting, and the corresponding connector fitting on the ventilator connector is a Luer-lock corresponding fitting, and such connection is achieved by rotation.

In some embodiments, the dispense into the ventilator connector according to any of the previous six embodiments is directed towards a direction of the patient.

In some embodiments, the ventilator connector according to any of the previous seven embodiments has an elbow configuration, and does not include an inner channel in proximity to its connector fitting.

In some embodiments, the patient according to any of the previous eight embodiments has a pulmonary disorder.

In some embodiments, the patient according to any of the previous nine embodiments has a pulmonary disorder, the pulmonary disorder includes COVID-19, the API comprises an anti-viral therapeutic agent for treating COVID-19, where the anti-viral therapeutic agent comprises HCQ, a free base thereof, or a pharmaceutically acceptable salt thereof.

In some embodiments, the pharmaceutical formulation according to any of the previous ten embodiments further includes: an alcohol of about 5% (w/w) of the pharmaceutical formulation; a propellant of about 95% (w/w) of the pharmaceutical formulation, where “w/w” denotes weight by weight.

In some embodiments, the pharmaceutical formulation according to any of the previous eleven embodiments further includes: an alcohol of about 5% (w/w) of the pharmaceutical formulation, where the alcohol is ethanol alcohol (“EtOH”); a propellant of about 94.6% (w/w) of the pharmaceutical formulation, where the propellant is HFA-134a, the HCQ is about 0.4% (w/w) of the pharmaceutical formulation, the HCQ is free base, the pharmaceutical formulation is a true solution, and the pharmaceutical formulation has a total weight of about 11.7 grams, and where “w/w” denotes weight by weight.

In some embodiments, the pharmaceutical formulation according to any of the previous twelve embodiments is an inhalable steroid.

In some embodiments, the inhalable steroid according to the previous embodiment is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

In some embodiments, the pharmaceutical formulation according to any of the previous fourteen embodiments includes a bronchodilator.

In same embodiments, the bronchodilator according to the previous embodiment is selected from the group consisting of albuterol, levosalbutamol, pirbuterol, epinephrine, racemic epinephrine, ephedrine, terbutaline, salmeterol, formoterol, bambuterol, indacaterol and pharmaceutically acceptable salts thereof.

In some embodiments, the pulmonary disorder according to any of the previous sixteen embodiments is selected from the group consisting of asthma, chronic obstructive pulmonary disease (COPD), sarcoidosis, eosinophilic pneumonia, pneumonia, interstitial lung disease, bronchiolitis, bronchiectasis, and restrictive lung diseases.

Some embodiments are directed to a pharmaceutical formulation for treating a pulmonary disease, including: an API for treating a pulmonary disease; a propellant, where the API is dissolved in the propellant at a pre-determined ratio, with or without a co-solvent, and wherein the pharmaceutical formulation is for administration by inhalation.

In some embodiments, the API according to the previous embodiment includes HCQ, a free base thereof, or a pharmaceutically acceptable salt thereof; and the propellant includes HFA 134a.

In some embodiments, the HCQ according to the previous embodiment is 0.25% to 1.50% (w/w); the propellant is 80.00% to 97.00% (w/w), where “w/w” denotes weight by weight, and is based on a total weight of the pharmaceutical formulation.

In some embodiments, the HCQ according to either of the previous two embodiments includes HCQ free base, and is 0.25% to 1.50% (w/w); the propellant includes HFA 134a, and is 80.00% to 97.00% (w/w), where “w/w” denotes weight by weight, and is based on a total weight of the pharmaceutical formulation; and the formulation is a true solution.

In some embodiments, the HCQ according to any of the previous three embodiments includes HCQ free base, and is 0.40% to 0.50% (w/w); the alcohol includes ethanol, and is 4.00% to 8.000% (w/w); the propellant includes HFA 134a, and is 93.00% to 96.00% (w/w); wherein “w/w” denotes weight by weight, and is based on a total weight of the pharmaceutical formulation; and the formulation is a true solution.

In some embodiments, the formulation according to any of the previous four embodiments further includes a co-solvent.

In some embodiments, the formulation according to any of the previous five embodiments further includes: a co-solvent; HCQ, a free base thereof, or a pharmaceutically acceptable salt thereof; and the propellant includes HFA 134a.

In some embodiments, the formulation according to any of the previous six embodiments includes: a co-solvent including alcohol, where the HCQ is 0.25% to 1.50% (w/w), where the alcohol is 3.00% to 15.00% (w/w), where the propellant is 80.00% to 97.00% (w/w), and where “w/w” denotes weight by weight, and is based on a total weight of the pharmaceutical formulation; and the formulation is a true solution.

In some embodiments, the formulation according to any of the previous seven embodiments includes: a co-solvent including alcohol, where the HCQ includes HCQ free base, where the HCQ free base is 0.25% to 1.50/o (w/w), where the alcohol includes ethanol, and the ethanol is 3.00% to 15.00% (w/w), where the propellant includes HFA 134a, and where the HFA 134a is 80.00% to 97.00% (w/w), where “w/w” denotes weight by weight, and is based on a total weight of the pharmaceutical formulation, and the formulation is a true solution.

In some embodiments, the formulation according to any of the previous eight embodiments further includes: a co-solvent including alcohol, where the HCQ includes HCQ free base, and is 0.40% to 0.50% (w/w), where the alcohol includes ethanol, and is 4.00% to 8.00% (w/w), and where the propellant includes HFA 134a, and is 93.00% to 96.00% (w/w), where “w/w” denotes weight by weight, and is based on a total weight of the pharmaceutical formulation; and the formulation is a true solution.

In some embodiments, the co-solvent according to any of the previous eight embodiments is about 4.00% (w/w), about 4.50% (w/w), about 5.00% (w/w), about 5.50% (w/w), about 6.00% (w/w), about 8.00% (w/w), or about 12.00% (w/w).

In some embodiments, the co-solvent according to any of the previous nine embodiments includes alcohol, the alcohol comprises ethanol, and ethanol is about 4.00% (w/w), about 4.50% (w/w), about 5.00% (w/w), about 5.50% (w/w), about 6.00% (w/w), about 8.00% (w/w), or about 12.00% (w/w).

In some embodiments, the co-solvent according to any of the previous ten includes alcohol, the alcohol includes ethanol, and ethanol is about 5.00% (w/w).

In some embodiments, the HCQ according to any of the previous eleven embodiments is about 0.38% (w/w), about 0.44% (w/w), about 0.54% (w/w), about 0.60% (w/w), about 0.76% (w/w), or about 1.08% (w/w).

In some embodiments, the HCQ according to any of the previous twelve embodiments includes HCQ free base.

In some embodiments, the HCQ according to any of the previous thirteen embodiments includes HCQ free base, and HCQ free base is about 0.43% (w/w).

In some embodiments, the propellant according to any of the previous fourteen embodiments is about 86.92% (w/w), about 91.24% (w/w), about 93.40% (w/w), about 94.06% (w/w), about 94.46% (w/w), about 94.56% (w/w), about 94.57% (w/w), about 94.62% (w/w), about 95.06% (w/w), or about 95.62% (w/w).

In some embodiments, the propellant according to any of the previous fifteen embodiments includes HFA 134a.

In some embodiments, the propellant according to any of the previous sixteen embodiments includes HFA 134a, and HFA 134s is about 94.57% (w/w).

In some embodiments, the pulmonary disease according to any of the previous seventeen embodiments includes a pulmonary disease capable of infecting a plurality of the alveoli in at least one lung of a patient.

In some embodiments, the pulmonary disease according to any of the previous eighteen embodiments includes COVID-19, and COVID-19 includes a pulmonary disease capable of infecting a plurality of the alveoli in at least one lung of a patient.

In some embodiments, the pharmaceutical formulation according to any of the previous nineteen embodiments is in a metered-dose inhaler (“MDI”).

In some embodiments, the MDI according to the previous embodiment is capable of dispensing, per actuation, a metered-dose of the anti-viral agent of 0.05 mg to 1.00 mg.

In some embodiments, the MDI according to the previous two embodiments is capable of dispensing, per actuation, a metered-dose of the anti-viral agent of about 0.175 mg, about 0.2 mg, about 0.205 mg, about 0.25 mg, about 0.275 mg, or about 0.5 mg.

In some embodiments, the MDI according to the previous three embodiments includes a metered-dose of the anti-viral agent of about 0.2 mg.

In some embodiments, the total weight of the pharmaceutical formulation according to any of the previous twenty-three embodiments is about 5-15.0 grams.

In some embodiments, the total weight of the pharmaceutical formulation according to any of the previous twenty-four embodiments is about 8-12 grams.

In some embodiments, the formulation according to any of the previous twenty-five embodiments includes an inhalable steroid.

In some embodiments, the inhalable steroid according to the previous embodiment is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

In some embodiments, the formulation according to any of the previous twenty-seven embodiments includes a bronchodilator.

In some embodiments, the bronchodilator according to the previous embodiment is selected from the group consisting of albuterol, levosalbutamol, pirbuterol, epinephrine, racemic epinephrine, ephedrine, terbutaline, salmeterol, formoterol, bambuterol, indacaterol and pharmaceutically acceptable salts thereof.

In some embodiments, the formulation according to any of the previous twenty-nine embodiments further includes a surfactant.

In some embodiments, the surfactant according to the previous embodiment includes one of polyethylene glycol, brij, polysorbate, polypropylene glycol, a poloxamer, polyvinyl pyrrolidone, polyvinyl alcohol, sodium dioctyl sulfosuccinate, oleic acid, oligolactic acid, lecithin, or span.

In some embodiments, wherein the surfactant according to either of the previous two embodiments includes a poloxamer.

Some embodiments are directed to an aerosol formulation capable of being delivered by an MDI, the formulation including: HCQ, a free base thereof, or a pharmaceutically acceptable salt thereof; a propellant including one or more HFAs, or a mixture thereof; and a co-solvent, where the co-solvent includes an alcohol, the alcohol includes ethanol, and the co-solvent is in an amount effective to solubilize the HCQ in the propellant.

In some embodiments, the HCQ according to the previous embodiment is about 0.30% (w/w) to about 0.75% (w/w), where w/w denotes weight by weight, and is based on a total weight of the formulation.

In some embodiments, the ethanol according to either of the previous two embodiments is about 2% (w/w) to about 12% (w/w), where w/w denotes weight by weight, and is based on a total weight of the formulation.

In some embodiments, the propellant according to any of the previous three embodiments is about 90% (w/w) to about 98% (w/w), where w/w denotes weight by weight, and is based on a total weight of the formulation.

In some embodiments, the propellant according to any of the previous four embodiments includes one or more HFAs, or a mixture thereof, wherein the one or more HFAs is selected from the group of HFA-134a and HFA-227.

In some embodiments, the HCQ according to any of the previous five embodiments is HCQ in free base, the formulation is a true solution, where HCQ is about 0.43% (w/w), where ethanol is about 5% (w/w), where the propellant includes HFA 134a, and the propellant is 94.57% (w/w), where w/w denotes weight by weight, and is based on a total weight of the formulation.

In some embodiments, formulation according to any of the previous six embodiments has particle distribution which allows delivery of an effective dose of the HCQ to the upper and lower respiratory tracts, including a significant amount of super-fine HCQ particles that are capable of reaching to a deep portion of a lung of a patient where a plurality of alveoli are located.

In some embodiments, the super-fine HCQ particles according to the previous embodiment has an appreciable portion delivered to Stages 6, 7 and filter, as those defined by a Cascade Impactor for a particle size distribution of a respiratory track.

In some embodiments, a nozzle of an MDI actuator for use for the MDI according to the previous eight embodiments has an inner diameter of 0.42 μm to 0.18 μm, thereby producing desired sizes of HCQ particles for effective delivery to a deep portion of a lung of a patient where a plurality of alveoli are located.

In some embodiments, an inner diameter of the nozzle according to the previous embodiment is from 0.25 mm to 0.18 mm.

Some embodiments are directed to a method for deep-lung targeted delivery of an anti-viral therapeutic agent for treating a pulmonary disease, the method including: administering, as an inhalation using a MDI actuator, one or more metered doses of a pharmaceutical formulation to a patient having a pulmonary disease, where a portion of the pharmaceutical formulation is administered to a deep portion of a lung of the patient where a plurality of alveoli are located, where the pharmaceutical formulation includes an API, where the API is for treating the pulmonary disease, and where a therapeutically effective amount of the API for treating the pulmonary disease is administered by one or more metered doses of the pharmaceutical formulation.

In some embodiments, the API according to the previous embodiment is capable of being delivered to a whole respiratory airway tract, including from an upper airway, a lower airway, and the plurality of alveoli in a deep portion of the patient's lungs in order to treat the pulmonary disease.

In some embodiments, the deep portion of the lung where the plurality of alveoli are located according to either of the previous two embodiments includes at least Stage 6 based on a Cascade Impactor particle size distribution of a respiratory track, where Stage 6 has a particle diameter of about 1.1 μm or less.

In some embodiments, the deep portion of the lung where the plurality of alveoli are located according to any of the previous three embodiments includes at least Stage 6 and Stage 7 based on a Cascade Impactor particle size distribution of a respiratory track, where Stage 6 and Stage 7 include a particle diameter of 0.4 μm to 1.1 μm.

In some embodiments, in a single metered dose according to any of the previous four embodiments, at least about 30% of the anti-viral therapeutic agent has a particle diameter of less than about 1.1 μm or less, and the at least about 30% of the anti-viral therapeutic agent is capable of being delivered to the deep portion of the lung where the plurality of alveoli and other portions of the patient's lung having a diameter of 1.1 μm to 4.7 μm.

In some embodiments, in a single metered dose according to any of the previous five embodiments, at least about 30% of the anti-viral therapeutic agent has a particle diameter of less than about 1.1 μm or less, and the at least about 30% of the anti-viral therapeutic agent is capable of being delivered as a dissolved API particle to a portion of an alveolar lining fluid, resulting in a relatively high local plasma concentration for treating the pulmonary disease.

In some embodiments, in a single metered dose according to any of the previous six embodiments, at least about 30% of the anti-viral therapeutic agent has a particle diameter of less than about 1.1 μm or less, and the at least about 30% of the anti-viral therapeutic agent is capable of being delivered to the deep portion of the lung where the plurality of alveoli and other portions of the patient's lung having a diameter of 1.1 μm to 4.7 μm, and capable of being delivered as dissolved API particles to a portion of an alveolar lining fluid, resulting in a relatively high local plasma concentration for treating the pulmonary disease.

In some embodiments, the administration according to any of the previous seven embodiments has a deep-lung delivery efficiency rate of at least 30.0% per actuation, wherein the deep-lung delivery efficiency rate is determined by dividing (i) a total amount, per actuation, of the anti-viral therapeutic agent having particles with a diameter of less than 1.1 μm, by (ii) a single metered dose of the anti-viral therapeutic agent, and the deep-lung delivery efficiency rate shows the delivery efficiency of API particles to be delivered to portions of the patient's lung having a diameter of 1.1 μm or less, and 1.1 μm to 4.7 μm.

In some embodiments, the therapeutically effective dose of the anti-viral therapeutic agent according to any of the previous eight embodiments is intended for substantially non-systemic delivery to lower systemic exposure of the anti-viral therapeutic agent, and cause less adverse drug events (“ADE”) compared to a same or a different anti-viral therapeutic agent using a different route of administration.

In some embodiments, the therapeutically effective dose of the anti-viral therapeutic agent according to any of the previous nine embodiments is intended for substantially non-systemic delivery to lower systemic exposure of the anti-viral therapeutic agent, and lower risk of overdose toxicity compared to a same or a different anti-viral therapeutic agent using a different route of administration.

In some embodiments, the lower systemic exposure of the anti-viral therapeutic agent according to either of the previous two embodiments is compared to an oral administration of a tablet comprising an API, wherein the API is HCQ or chloroquine (“C”).

In some embodiments, the anti-viral therapeutic agent according to any of the previous eleven embodiments is hydroxychloroquine (“HCQ”), in a free base thereof, or a pharmaceutically acceptable salt thereof.

In some embodiments, a single metered dose according to any of the previous twelve embodiments, per actuation, is 0.05 mg to 1.00 mg of the anti-viral therapeutic agent. In some embodiments, a single metered dose, per actuation, is about 0.20 mg of the anti-viral therapeutic agent.

In some embodiments, the pulmonary disease according to any of the previous thirteen embodiments is a pulmonary disease that is capable of infecting a plurality of alveoli in at least one lung of the patient.

In some embodiments, the pulmonary disease according to any of the previous fourteen embodiments includes COVID-19, where COVID-19, via a SARS-CoV-2 virus, is capable of infecting a plurality of alveoli in at least one lung of the patient.

In some embodiments, the patient according to any of the previous fifteen embodiments has at least mild COVID-19, and the therapeutically effective dose is 0.4 mg to 3.0 mg of the anti-viral therapeutic agent.

The method of claim 174, the patient according to any of the previous sixteen embodiments has at least mild COVID-19, and the pharmaceutical formulation can be self-administered using a handheld MDI actuator having an nozzle with an inner diameter of about 0.20-0.25 mm.

In some embodiments, the patient according to any of the previous seventeen embodiments has at least mild COVID-19, and the therapeutically effective dose is about 1.0 to 2.0 mg of the anti-viral therapeutic agent. In some embodiments, the patient has severe COVID-19, and the therapeutically effective dose is 0.8 mg to 4.0 mg of the anti-viral therapeutic agent. In some embodiments, the patient has severe COVID-19, and the therapeutically effective dose is about 1.0-3.0 mg of the anti-viral therapeutic agent.

In some embodiments, the patient according to any of the previous eighteen embodiments is treated with the claimed doses 2-6 times per day. In some embodiments, the patient is treated with the claimed 3 to 12 days.

In some embodiments, the patient according to any of the previous nineteen embodiments is operatively connected to a ventilator, and the MDI actuator is capable of ventilator-delivery of the anti-viral therapeutic agent to the patient via ventilator circuitry. In some embodiments, the patient has severe COVID-19 but is on non-invasive airway support, and the pharmaceutical formulation can be self-administered using a handheld MDI actuator having a nozzle with an inner diameter of about 0.20-0.25 mm.

In some embodiments, a closed ventilator circuitry system is maintained without disruption during administration of the one or more metered doses of the pharmaceutical formulation according to any of the previous nineteen embodiments to the patient operatively connected to the ventilator according to the previous embodiment.

In some embodiments, the pharmaceutical formulation according to any of the previous twenty-one embodiments further includes: HCQ that is 0.25% to 1.50% (w/w); an alcohol of 3.00% to 15.00% (w/w); a propellant of 80.00% to 97.00% (w/w), where “w/w” denotes weight by weight.

In some embodiments, the pharmaceutical formulation according to any of the previous twenty-two embodiments further includes: HCQ that is 0.25% to 1.50% (w/w); an alcohol of 3.00% to 15.00% (w/w), the alcohol is ethanol; a propellant of 80.00% to 97.00% (w/w), the propellant is HFA 134a, where “w/w” denotes weight by weight.

In some embodiments, the pharmaceutical formulation according to any of the previous twenty-three embodiments further includes: HCQ that is HCQ free base and is 0.35% to 0.60% (w/w), where the alcohol is ethanol, and is 4.00% to 8.00% (w/w), where the propellant is HFA 134a, and is 93.00% to 96.00% (w/w), and where “w/w” denotes weight by weight and the formulation is a true solution.

In some embodiments, the pharmaceutical formulation according to any of the previous twenty-four embodiments further includes: a propellant, where the propellant is HFA 134a, and where the HCQ is dissolved in the HFA 134a at a pre-determined ratio, with or without a co-solvent.

In some embodiments, the pharmaceutical formulation according to any of the previous twenty-five embodiments includes an inhalable steroid.

In some embodiments, the inhalable steroid according to the previous embodiment is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

In some embodiments, the pharmaceutical formulation according to any of the previous twenty-seven embodiments includes a bronchodilator.

In some embodiments, the bronchodilator according to the previous embodiment is selected from the group consisting of albuterol, levosalbutamol, pirbuterol, epinephrine, racemic epinephrine, ephedrine, terbutaline, salmeterol, formoterol, bambuterol, indacaterol and pharmaceutically acceptable salts thereof.

In some embodiments, the pulmonary disease according to any of the previous twenty-eight embodiments is selected from the group consisting of asthma, chronic obstructive pulmonary disease (COPD), sarcoidosis, eosinophilic pneumonia, pneumonia, interstitial lung disease, bronchiolitis, bronchiectasis, and restrictive lung diseases.

Some embodiments are directed to an aerosol drug delivery device having a dual role as a MDI actuator and an adaptor to a ventilator circuit for administering inhalation pharmaceutical medications to a mechanically ventilated patient and provides particle size control of the aerosol product to enable delivery of the medication to a desired target site with airtight connection and virus mitigating capability.

In some embodiments, the device according to the previous embodiment includes a housing with cylindrical “cup” for containing an MDI and two finger grips to be hand-held by a user.

In some embodiments, the device according to either of the previous two embodiments includes a stem extruded from both side of the “cup” floor, of which the inward extrusion has recess to mate with valve stem of the MDI, and the outward extrusion tip tapered out and has an actuator nozzle in the center.

In some embodiments, the device according to any of the previous three embodiments includes an adaptor having a Luer-lock connector extruded from outward of the “cup” floor for an airtight connecting to the ventilator circuit.

In some embodiments, the device according to any of the previous four embodiments eliminates the aerosolization of a virus through the connection between the device and the ventilator circuit due to the Luer-lock connection providing an airtight, virus mitigating connection.

In some embodiments, the inhalation pharmaceutical medication according to any of the previous five embodiments is for combating COVID-19 virus and/or other viral infectious diseases.

In some embodiments, the API of the inhalation pharmaceutical medication according to any of the previous six embodiments is (i) hydroxychloroquine (“HCQ”), (ii) HCQ free base, or (iii) a pharmaceutically acceptable salt of HCQ.

In some embodiments, the inhalation pharmaceutical medication according to any of the previous seven embodiments is toxic, including oncology, cytotoxic medications, and chemotherapeutic medications, which may be harmful to ambient environment and health care professionals who is administering the medication to mechanically ventilated patients.

In some embodiments, the device according to any of the previous eight embodiments can maintain a target-site delivery efficiency up to 80% via ventilator delivery as compared to that of using a MDI without a ventilator.

In some embodiments, an add-on dose counter can be used in order to predict a quantity of remaining metered-doses of the inhalation pharmaceutical medication in the MDI unit according to any of the previous nine embodiments.

In some embodiments, the device according to any of the previous ten embodiments provides the particle size control of the aerosol product by producing fine particles having particle diameter of less than 4.7 μm, and extra-fine particles having particle diameter of less than 1.1 μm.

In some embodiments, the device according to any of the previous eleven embodiments provides a highly efficient delivery comprising: a delivery efficiency of no less than 60% of the fine API particles to the respiratory tract; and a delivery efficiency of no less than 30% of the extra-fine API particles to the deep, peripheral lungs, alveoli, or alveoli lining fluid.

In some embodiments, the MDI actuator/adaptor according to any of the previous twelve embodiments possesses a structure which is capable of sealing the gap between MDI canister and the actuator/adaptor, which seamlessly blocks the aerosol that mixes the virus or bacteria particles exhaled by patients and the pharmaceutical product aerosol escaped from the transfer hole on MDI valve stem.

In some embodiments, the sealing structure according to the previous embodiment is any materials in any shape that is capable of sealing the gap between MDI canister and the actuator/adaptor, such that the leaking limit is controlled to under the desired limit, which depends on the size of the virus to be protected against.

In some embodiments, the sealing structure according to either of the previous two embodiments is a single elastic ring made of Silicone rubber (SiR), Nitrile rubber (NBR, Buna-N), Ethylene propylene diene monomer (EPDM), Ethylene propylene rubber (EPR), Polychloroprene (neoprene), Polytetrafluoroethylene (PTFE), Polyisoprene (IR), Butyl rubber (IIR), Polyacrylate rubber (ACM), Butadiene rubber (BR), Sanifluor (FEPM), Fluoroelastomer (FKM), Fluoroelastomer (FKM), Perfluoroelastomer (FFKM), Polysulfide rubber (PSR), Styrene-butadiene rubber (SBR), chlorosulfonated polyethylene (CSM), or blends thereof. In some embodiments, the sealing structure is a washer shaped elastic film made of Silicone rubber (SiR), Nitrile rubber (NBR, Buna-N), Ethylene propylene diene monomer (EPDM), Ethylene propylene rubber (EPR), Polychloroprene (neoprene), Polytetrafluoroethylene (PTFE), Polyisoprene (IR), Butyl rubber (IIR), Polyacrylate rubber (ACM), Butadiene rubber (BR), Sanifluor (FEPM), Fluoroelastomer (FKM), Fluoroelastomer (FKM), Perfluoroelastomer (FFKM), Polysulfide rubber (PSR), Styrene-butadiene rubber (SBR), chlorosulfonated polyethylene (CSM), or blends thereof.

In some embodiments, the MDI actuator/adaptor according to any of the previous fifteen embodiments possesses the leak proof protection that prevent toxic medications from escaping to ambient environment and protect health care professionals who is administering the medication to mechanically ventilated patients.

In some embodiments, the MDI actuator/adaptor according to any of the previous sixteen embodiments possesses the virus mitigating protection to the medical professionals taking care of mechanically ventilated patients who have highly contagious viral infection diseases, such as COVID-19.

### BRIEF DESCRIPTION OF THE DRAWINGS

The accompanying figures, which are incorporated herein, form part of the specification and illustrate embodiments of the present disclosure. Together with the description, the figures further serve to explain the principles of and to enable a person skilled in the relevant art(s) to make and use the disclosed embodiments. These figures are intended to be illustrative, not limiting. Although the disclosure is generally described in the context of these embodiments, it should be understood that it is not intended to limit the scope of the disclosure to these particular embodiments. In the drawings, like reference numbers indicate identical or functionally similar elements.

FIG. 1 is a graph depicting the plasma concentration of HCQ from an HCQ oral tablet treatment regimen as a function of time.

FIG. 2 is an illustration demonstrating the stages for Cascade Impactor mass distribution along the human respiratory airway.

FIG. 3 is an illustration demonstrating the approximate fluid volume in a lung.

FIG. 4 is an illustration depicting a process of combating a virus within the alveoli according to some embodiments.

FIG. 5A is a side view of an MDI actuator configured for stand-alone use according to some embodiments.

FIGS. 5B and 5C are exploded views of the MDI actuator of FIG. 5A.

FIG. 5D is a cross-sectional view of the MDI actuator of FIG. 5A.

FIG. 6A is a side view of an MDI actuator configured for use with an auxiliary delivery component according to some embodiments.

FIG. 6B is another side view of the MDI actuator of FIG. 6A.

FIG. 6C is an exploded view of the MDI actuator of FIG. 6A.

FIG. 7A is a cross-sectional side view of an MDI actuator configured for use with an auxiliary delivery component according to some embodiments.

FIG. 7B is a zoomed-in view of the MDI actuator body of FIG. 7A.

FIG. 7C is a bottom view of the MDI actuator body of FIG. 7A.

FIG. 7D is a bottom perspective view of the MDI actuator body of FIG. 7A.

FIG. 8A is a side view of an MDI actuator and ventilator device accordingly to some embodiments.

FIG. 8B is a cross-sectional side view of an MDI actuator and ventilator device according to some embodiments.

FIG. 9A is a side view of an MDI actuator including a pharmaceutical formulation according to some embodiments.

FIG. 9B is a zoomed-in view of the MDI actuator of FIG. 9A shown in an actuated state.

FIG. 9C is a zoomed-in view of the MDT actuator of FIG. 9A.

FIG. 10 shows side view of various MDI actuator inserts according to some embodiments.

FIG. 11 is a graph depicting the percentage of API particle diameter sizes having about 1.1 μm or less as a function of the inner diameter of a nozzle of MDI actuators according to some embodiments.

FIG. 12 is a graph depicting the HCQ concentration in alveoli fluid from administration of HCQ oral tablets versus the HCQ concentration in alveoli fluid from administration of a pharmaceutical composition via a MDI actuator configured for stand-alone use according to some embodiments.

FIG. 13 is a graph depicting the HCQ concentration in alveoli fluid from administration of HCQ oral tablets versus the HCQ concentration in alveoli fluid from administration of a pharmaceutical composition via a MDI actuator for use with an auxiliary delivery component according to some embodiments.

FIGS. 14A-14D are bar charts depicting the efficacy of MDI actuators configured for stand-alone use according to some embodiments.

FIGS. 15A-15F are bar charts depicting the efficacy of MDI actuators configured for use with an auxiliary delivery component according to some embodiments.

FIG. 16 is an illustration depicting the leak path in some ventilators utilizing MDI's.

FIG. 17A is a side view of an MDI actuator for use with an auxiliary delivery component according to some embodiments.

FIG. 17B is a side view of an MDI actuator for use with an auxiliary delivery component according to some embodiments.

FIG. 18 is a side view of a stainless steel breathing tank for a mouse study.

FIG. 19A is a table demonstrating an amount of HCQ in a mouse's lungs vs. time.

FIG. 19B is a graph demonstrating an amount of HCQ in a mouse's lungs vs. time.

FIG. 20A is a table demonstrating Andersen test results of an MDI actuator configured for use with an auxiliary delivery component according to some embodiments.

FIG. 20B is a table demonstrating Andersen test results of an MDI actuator configured for use with an auxiliary delivery component according to some embodiments.

### DETAILED DESCRIPTION

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure. Throughout the drawings, like reference numerals will be understood to refer to like elements, features and structures.

### The COVID-19 Pandemic

As discussed above, COVID-19 is an infectious disease caused by SARS-CoV-2 (“CoV2”). CoV2 spreads from person to person through respiratory droplets produced when an infected person coughs, sneezes, or talks. In March of 2020, the World Health Organization announced that the widespread transmission of COVID-19 had become a pandemic. As of mid-April of 2021, there were more than 138 million COVID-19 cases reported globally, with more than 31.5 million cases in the United States alone, which caused more than 564,000 deaths. To mitigate the spread of COVID-19, the United States Center for Disease Control (CDC) recommends that people wear masks in public settings, and when around people outside of their household, especially when other social distancing measures are difficult to maintain. Social distancing, also called “physical distancing,” means maintaining a safe distance, for example a distance of at least 6 feet (about 2 arm's lengths), from other people. Social distancing should be practiced in combination with other everyday preventive actions to reduce the spread of COVID-19.

Approximately 80% of people infected by COVID-19 are considered to be mild or moderate. However, in about 15% of cases, the immune system's response to inflammation in the lungs can cause what is known as a “cytokine storm” and such a reaction is considered to be severe. The common symptoms of COVID-19 include dry cough, difficulty breathing (e.g. shortness of breath), fever (e.g. body temperature of 100.4° Fahrenheit or higher), and fatigue. More severe cases of COVID-19 can cause patients to require a ventilator assistance, though, and in extreme cases, COVID-19 infections may result in death. Alternatively, some individuals infected with COVID-19 may be asymptomatic (e.g. displays no symptoms of COVID-19), but can still spread COVID-19 to others who may be more susceptible to infection.

### How CoV2 Enters the Human Body and Replicates

CoV2 typically enters the human body through the nose and/or mouth, and travels along the airway tract into the lungs. The inhaled Virus can bind to epithelial cells in the nasal cavity, where it begins to replicate. Once it reaches the lungs, CoV2 uses its distinctive spike-shaped proteins to “hijack” cells in the alveoli. When CoV2's RNA has entered a hijacked cell, new copies of CoV2 are made. This replication process kills the hijacked cell, which allows for the new copies of CoV2 to be released out of the hijacked cell to infect neighboring cells. CoV2's process of hijacking cells to reproduce causes inflammation in the lungs, which triggers an immune response. As this process unfolds, fluid begins to accumulate in the alveoli, causing a dry cough and making breathing difficult. This process can also cause severe alveolar damage, which is a major cause of morbidity and mortality in affected COVID-19 patients.

### Treatment of COVID-19 by HCQ/CQ Oral Tablets

Both HCQ and CQ oral tablets have been used for many years in the treatment and prevention of malaria as well as for chronic inflammatory diseases such as rheumatoid arthritis and systemic lupus erythematosus. Recently, HCQ and CQ oral tablets have also received much attention as potential therapies of COVID-19. Optimism for repurposing these drugs stems from two lines of evidence: inhibition of Coronaviridae (including SARS and SARS-CoV-2) in vitro, and preliminary off-label clinical data from studies conducted in the United States, China, and France. However, the effectiveness of HCQ oral tablets in treating COVID-19 has not been proven, and the tablets may have only limited effectiveness and may also present potential safety concerns.

First, with respect to effectiveness, the recommended HCQ dose using oral tablet treatment for COVID-19 is: Day-1 2×400 mg, Day-2 to 5, 400 mg, for a 5 day dose total dose of 2,400 mg. It has been reported that an oral HCQ tablet dose of 200 mg results in the Cmax=50.3 ng/mL in plasma. This Cmax corresponds to 0.113 μM in plasma, which is only 0.07% of the HCQ tablet dose.

In fact, according to this study, the majority (99.93%) of the HCQ tablet dose is distributed as follows: (i) approximately 0.25% is distributed to red blood cells or other protein in blood; (ii) approximately 73.7% is distributed to the tissues of the human body; and (iii) approximately 26% is not absorbed or initially metabolized in liver during absorption and initially passes through liver (BA=74%).

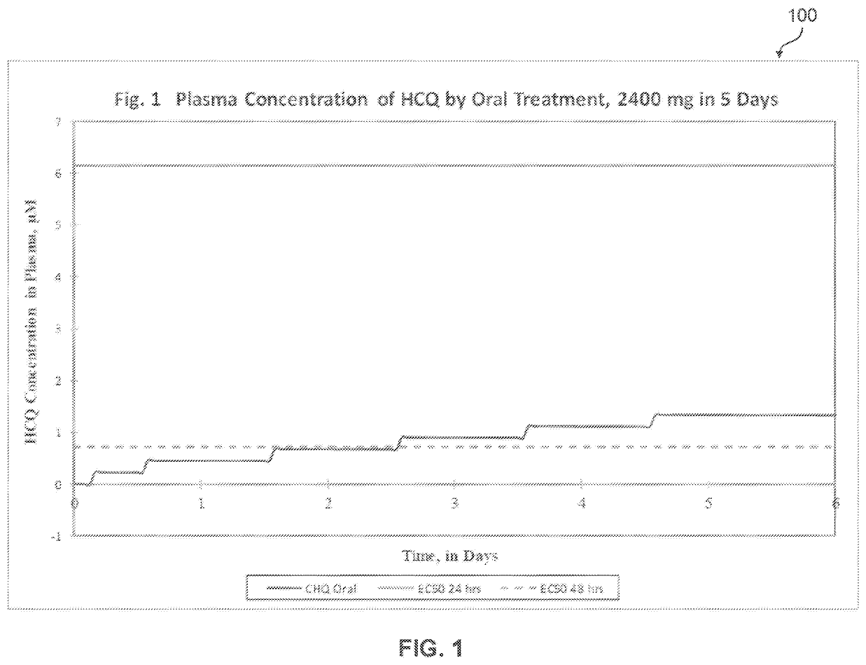

Therefore, only a low concentration, contributed by 0.07% of HCQ oral tablet dose, is generally distributed to the alveolar fluid via the plasma. This is illustrated, for example, in plot 100, shown in FIG. 1, which shows the concentration of HCQ in the plasma for 2400 mg of HCQ administered via oral tablet over a period of 5 days.

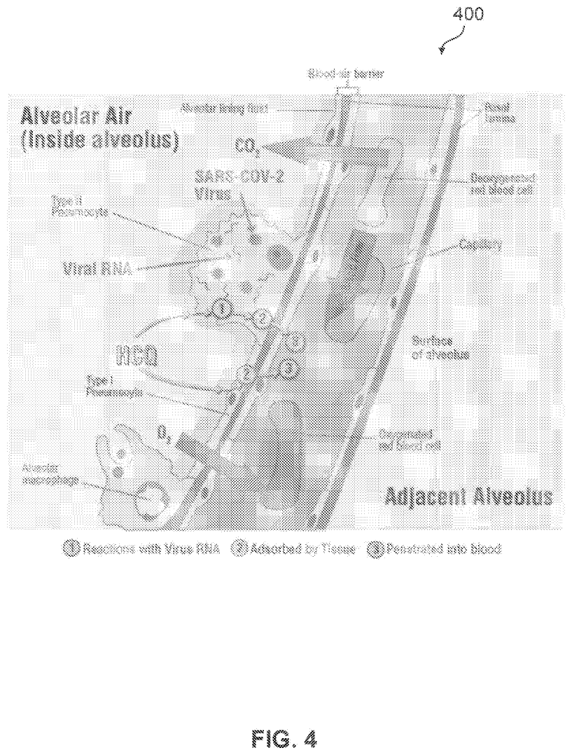

The HCQ molecules in the plasma can penetrate capillaries outside the alveolar membrane to reach the alveolar lining fluid (“ALF”). However, due to strong hydrogen bonds, a large percentage of HCQ molecules are held by the red blood cells and by tissues, and are not available to reach the liquid phase of the ALF, or the inside of the alveoli, for example as shown in illustration 400 in FIG. 4. Accordingly, the HCQ concentration in the ALF is typically no more than the HCQ concentration in the plasma. Thus, the HCQ concentration in the ALF is not expected to exceed the Cmax of HCQ in the plasma, because Cmax≥C(t) at any time t.

For example, over a 2400 mg oral dose of HCQ over a 5-day period, as shown in plot 100, the concentration of HCQ in the alveolar fluid is estimated to be 0.45 μM at Day-1 (800 mg dose) and 1.3 μM at Day-5 (total dose of 2,400 mg). The estimated curve in plot 100 for HCQ concentration in human plasma is based on (i) Cmax in plasma of HCQ with 200 mg oral tablet dose [30], (ii) the corresponding tmax, (iii) HCQ's half-life in human plasma [30], and (iv) dose used by the oral tablet treatment for COVID-19 in 5 days (2,400 mg). The known HCQ EC50 for inhibition of CoV2 is 6.14 μM for 24 hrs and 0.72 μM for 48 hrs. However, in first two treatment days, the HCQ concentrations (Day-1 0.23 and 0.45 μM after the 1st and 2nd 400 mg dose in Day-1, respectively, and Day-2 0.67 μM) are below the EC50s. This explains why the low HCQ concentration in alveolar fluid provided by HCQ oral tablets may be insufficient for effectively treating CoV2, and therefore likely suboptimal for anti-viral treatment against this Virus.

### Limitations and Disadvantages of Current Methods for Administering Inhaled HCQ

HCQ may be administered in other forms aside from oral tablets. For example, asthma has been treated through inhalation of HCQ particles. Disadvantageously, though, the HCQ particles that are typically inhaled to treat asthma are unable to travel to a deep portion of a patient's lungs, where a large quantity of the alveoli are located, because their particle size is too large (ranging from 2.1 μm to 3.3 μm).

The particle size of an inhalation drug may be measured by an instrument called a Cascade Impactor, which consists of multiple discs. The size of the discs is graduated to properly determine the size of the particulate matter at various stages of the Cascade Impactor, which each represent the drug delivery to different portions of the entire respiratory tract. The Cascade Impactor collects samples of the drug in a graduated manner on each disc such that the average particle size of the collected drugs can be measured for each stage.

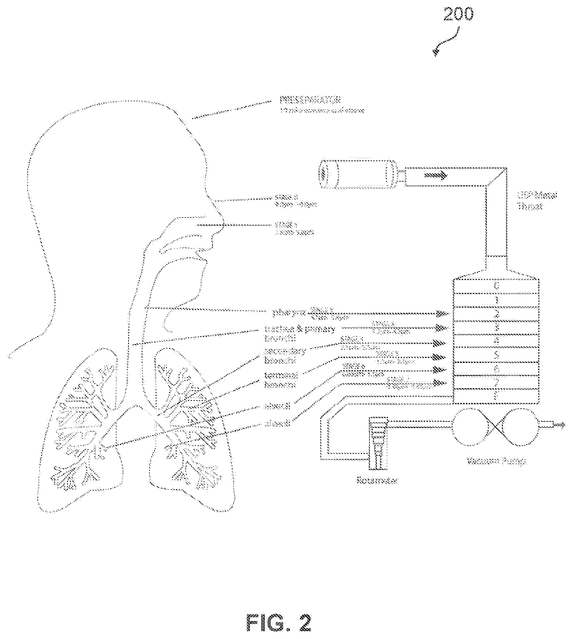

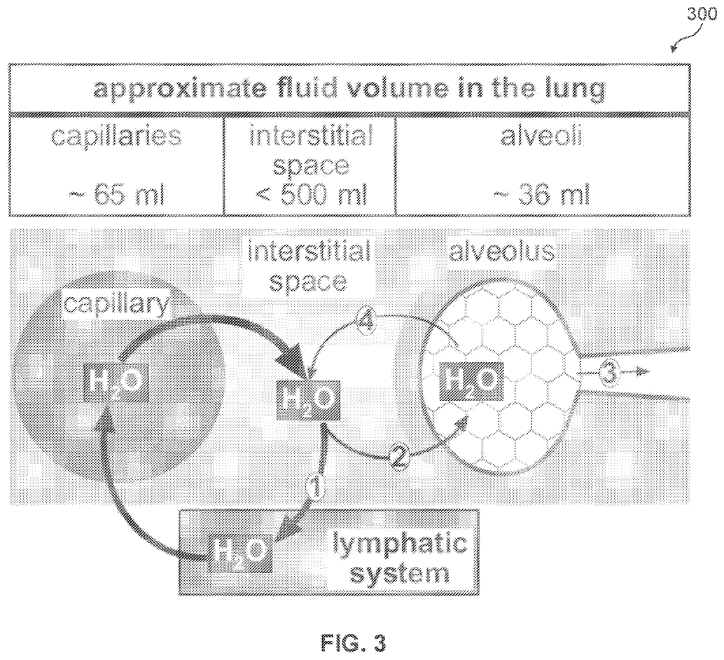

FIG. 2 shows an illustration 200 depicting Cascade Impactor results showing the maximum particle size measurements of various particles that may enter each portion of the respiratory tract. As shown in illustration 200, only particles having a particle size of about 1.1 μm or less can travel to Stage 6, which corresponds to a bottom portion of the lungs.

Thus, the inhaled HCQ particles that are typically used to treat asthma can only travel into the secondary bronchi, which corresponds to Stage 4. However, because of the close proximity of their mean of 3.2 μm HCQ particle size and the upper limit of Stage 4 being 3.3 μm, it is conceivable that some or even most of their HCQ particles are limited to Stage 3, where the trachea and primary bronchi are located. Thus, for pulmonary diseases that are capable of infecting the alveoli, such as COVID-19, this particle size is likely insufficient and ineffective.

Therefore, there is a critical unmet medical need to develop drug formulations and drug delivery products that overcome the aforementioned technical limitations and disadvantages of HCQ and CQ oral tablets and inhaled HCQ particles for treatment pulmonary diseases, such as COVID-19.

### Targeted Delivery MDI Configured for Stand-Alone Use for Administration of Pharmaceutical Formulations

As discussed above, there are limitations to oral administration of HCQ, making inhaled pharmaceutical formulations of the drug an appealing option for treatment of COVID-19. However, to reach the bottom portion of the lungs, the particle size of an inhaled HCQ formulation should not exceed about 1.1 μm. To achieve such a particle size, a handheld MDI actuator may be used to administer a spray of fine particles to achieve a drug delivery efficiency rate to the alveoli of at least 25.0%, wherein the delivery efficiency rate is determined by dividing (i) a total amount, per actuation, of an API having a certain particle size, such as less than about 1.1 μm, by (ii) an expected API dose per actuation.

Accordingly, disclosed herein are embodiments of MDI actuators configured to administer a spray of fine particles to achieve a drug delivery efficiency rate of at least 25.0%.

An MDI is a device that may deliver a metered dose of a pharmaceutical formulation, containing the dosage amount of an API per actuation (or per spray), into a patient's mouth, which may be inhaled into the patient's lungs. The MDI may administer the API in the form of a short burst of aerosolized spray. In an MDI, the pharmaceutical formulation is typically contained in a pressurized canister, such as an aluminum canister. The pharmaceutical formulation may include a propellant, for example CFC-free propellant hydrofluoroalkane (“HFA”), in order to drive the pharmaceutical formulation from the canister and dispense, per actuation, as an aerosolized spray suitable for inhalation. As used herein HFA may include HFA-134a, HFA-227, or any other pharmaceutically acceptable hydrofluoroalkane suitable for inhalation administration. The canister can be configured to dispense, per actuation or per spray, a metered dose of the pharmaceutical formulation. The metering function of the MDI may be configured to track the number of doses dispensed from the MDI, or the number of doses left in the MDI.

MDI's are commonly designed to allow for self-administration of an API through use of a handheld MDI actuator. Such self-administrable, handheld MDI actuators are often used as delivery systems for treating asthma, chronic obstructive pulmonary disease (“COPD”), and other respiratory diseases. The medications typically used in MDI's may be bronchodilator, corticosteroid or a combination of both for the treatment of asthma and COPD. Other medications less commonly used but also administered by MDI are mast cell stabilizers, such as cromoglicate or nedocromil. Thus, a pharmaceutical formulation for treatment of COVID-19 can also be self-administered using a handheld MDI actuator.

In some embodiments of the MDI actuators disclosed herein, the MDI actuator is capable of providing a highly efficient delivery of a pharmaceutical formulation to a portion of the patient's lung where a plurality of alveoli located. More particularly, the MDI actuators may be capable of providing a highly efficient delivery of the API particles, such as HCQ particles, having a particle diameter of about 1.1 μm or less, to a portion of the patient's lung where a plurality of alveoli located. Thus, in some embodiments, the portion of the lung where the plurality of alveoli are located may be in at least Stage 6 based on a Cascade Impactor particle size distribution of a respiratory tract, for example as outlined in FIG. 2, where Stage 6 has a particle diameter size of about 1.1 μm or less. In some embodiments, the portion of the lung where the plurality of alveoli are located includes at least Stage 6 and Stage 7, where Stage 6 and Stage 7 include a particle diameter size in a range of 0.43 μm to 1.1 μm.

Accordingly, in some embodiments, the disclosed MDI actuators are capable of providing a delivery efficiency rate of at least 25.0%, wherein the delivery efficiency rate is determined by dividing (i) a total amount, per actuation, of an API having a certain particle size, by (ii) an expected API metered dose per actuation. In some embodiments, the delivery efficiency rate is at least 30.0%, at least 35.0%, at least 40.0%, at least 45.0%, at least 50.0%, or more.

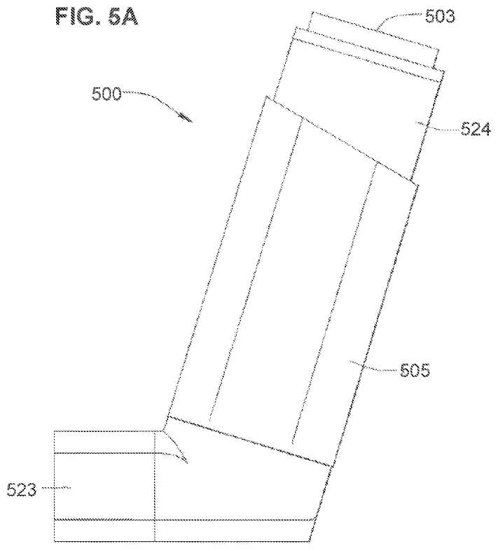

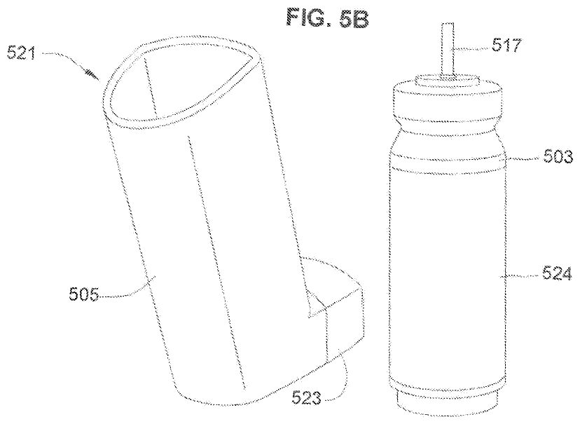

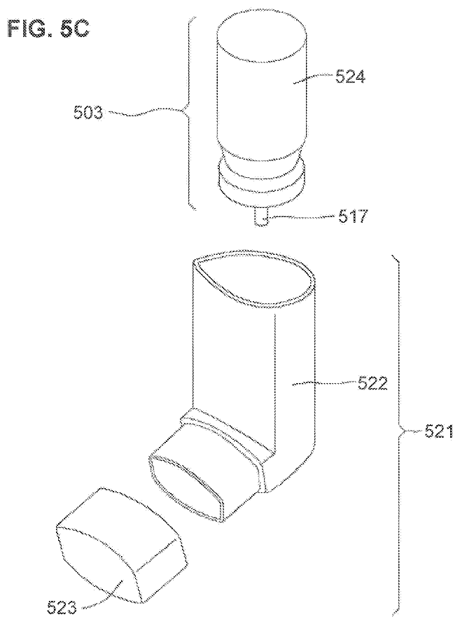

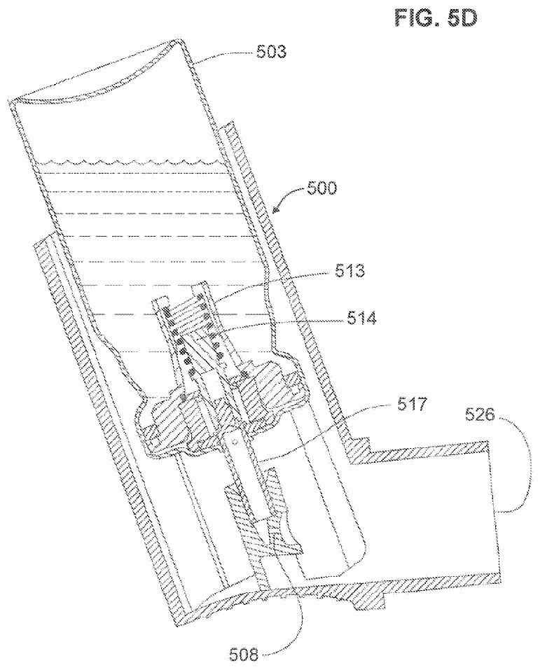

FIGS. 5A-5D show an MDI actuator 500 for an MDI 503, which includes body 505 and cap 523, which may cover the mouthpiece of MDI actuator 500 and provide protection of the mouthpiece. MDI actuator 500 may be configured for stand-alone use, such as a self-administrable, handheld MDI actuator.

In some embodiments, MDI 503 includes a canister 524 and a stem 517. Canister 524 may be a pressurized aluminum canister capable of storing a pharmaceutical formulation, for example HCQ, and may be capable of dispensing, per actuation (e.g. per spray) using MDI actuator 500, a metered-dose of the pharmaceutical formulation.

A pharmaceutical formulation is a formulation that includes at least one active pharmaceutical ingredient (“API”). In some embodiments, the pharmaceutical formulation is suitable for inhalation. Pharmaceutical formulations suitable for inhalation are pharmaceutical formulations that are intended to be administered to a patient by inhalation, such as being inhaled through a patient's mouth and into the patient's respiratory tract. For brevity, a pharmaceutical formulation suitable for inhalation is referred to herein as “inhalation pharmaceutical formulation.” A pharmaceutical formulation suitable for inhalation may additionally include a propellant, such as hydrofluoroalkane (“HFA”).

The disclosed pharmaceutical formulations may include various pharmaceutically acceptable excipients, as described herein. “Pharmaceutically acceptable” refers to an ingredient in the pharmaceutical formulation that is compatible with the other ingredients in the formulation, and does not cause excess harm to the patient receiving the pharmaceutical formulation.

In some embodiments, the MDI actuator is suitable for use with pharmaceutical formulations in which the API is suitable for inhalation delivery, including, but not limited to, hydroxychloroquine (“HCQ”), chloroquine (“CQ”), epinephrine, beclomethasone, albuterol, ipratropium, in a free base of any of the foregoing, the pharmaceutically acceptable salts of any of the foregoing, or any combination thereof. In some embodiments, the MDI actuator is suitable for use with a pharmaceutical formulation that is indicated for the treatment or prophylaxis of a pulmonary disease, such as COVID-19. In some embodiments, the API includes an anti-viral therapeutic agent, such as HCQ, in a free base thereof, or the pharmaceutically acceptable salts thereof. In some embodiments, the anti-viral therapeutic agent is capable of being delivered throughout a respiratory tract, including the upper and lower respiratory tract, and peripheral, deep lungs where alveoli are located.

As shown in FIG. 5A, MDI 503 may be aligned within body 505 in order to effectuate a spray, using MDI actuator 500, of a metered dose of an API, for example HCQ, within MDI 503. More particularly, body 505 may be capable of aligning stem 517 of MDI 503 to the functional, mechanical components inside of MDI actuator 500 that may be configured to actuate the pharmaceutical formulation from MDI 503.

FIG. 5D is a cross-sectional view of actuator 500, which shows the various functional, mechanical components inside of MDI actuator 500. These components are generally known in the art and thus, do not need to be described in detail. Briefly, MDI actuator 500 includes nozzle 508, mouthpiece 526, stem 517, spring 513, and buffer 514. Pharmaceutical formulations are dispensed, as an actuation (or spray), out of the nozzle 508, through mouthpiece 523, into the patient's mouth, and eventually traveling through the respiratory tract to the patient's lungs.

In some embodiments, body 523 can accommodate and align an MDI 403 having a canister having an outer diameter in a range of 20.0 mm to 25.0 mm, such as from 22.0 mm to 23.0 mm, about 22.0 mm, about 22.5 mm, or about 23.0 mm.

In some embodiments, body 505 has an inner diameter that is substantially circular to cooperate with a substantially circular outer diameter of canister 524. In some embodiments, body 505 has a substantially circular inner diameter in the range of 20.0 mm to 25.0 mm, including subranges, such as 21.0 mm to 24.0 mm, or 22.0 mm to 23.0 mm. In some embodiments, body 505 has an inner diameter of about 22.0 mm, about 22.5 mm, or about 23.0 mm.

Additionally, in some embodiments body 505 has a vertical length that covers at least a portion of canister 524. For example, body 505 may have a vertical length that covers at least 25%, at least 30%, at least 35%, at least 40%, at least 45%, at least 50%, at least 55%, at least 60%, at least 65%, or at least 70% of canister 524 with respect to the vertical length of canister 524 while it is in a non-actuated state. When canister 524 is in an actuated state (e.g., when canister 524 is pushed down into the actuator 500 in order to administer the drug), then body 505 may cover more of the vertical length of canister 524, such as at least 1% more, at least 2% more, at least 3% more, at least 4% more, at least 5% more, at least 6% more, at least 7% more, at least 8% more, at least 9% more, at least 10% more, or higher.

In some embodiments, body 505 may has one or more ribs to accommodate canister 524. In some embodiments, body 505 includes 2, 3, 4, or more ribs. In some embodiments, the one or more ribs are in the shape of substantially vertical columns.

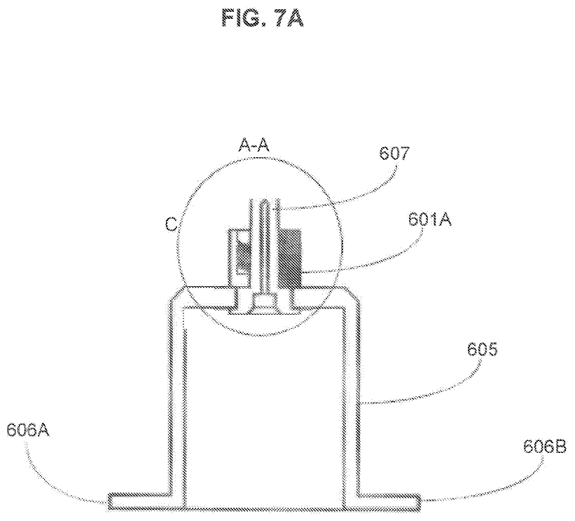

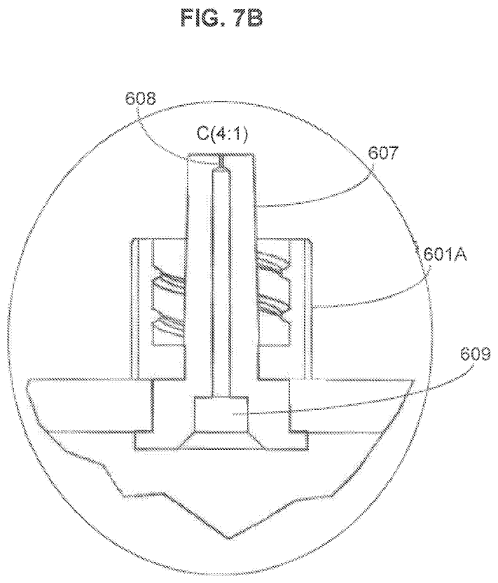

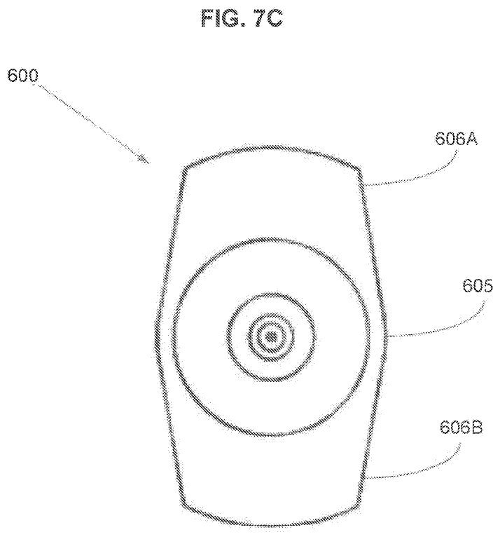

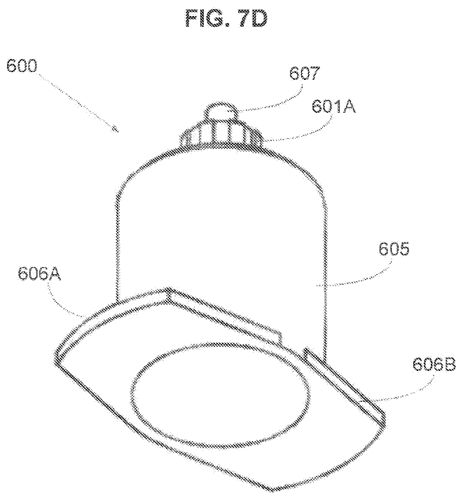

In some embodiments, MDI actuator 500 includes a nozzle, for example nozzle 508 shown in FIG. 7B, with an inner diameter of about 0.20 mm in order to dispense fine API particle sizes, such as API particles having a diameter of about 1.1 μm or less. In some embodiments, nozzle 508 has an inner diameter of 0.25 mm or less. For example, nozzle 508 may have an inner diameter in a range of 0.15 mm to 0.25 mm, including subranges, for example 0.16 mm to 0.24 mm, 0.17 mm to 0.23 mm, 0.18 mm to 0.22 mm, and 0.19 mm to 0.20 mm. In some embodiments, nozzle 508 has an inner diameter of about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, or 0.25 mm.

In some embodiments, in a single metered dose, at least about 40% of the API has a particle diameter of less than about 1.1 μm or less, and the at least about 40% of the API is capable of being delivered to a deep portion of the lung where a plurality of alveoli are located.

In some embodiments, in a single metered dose, at least about 40% of the API has a particle diameter of about 1.1 μm or less, and the at least about 40% of the API is capable of being delivered to as dissolved API particles to a portion of an alveolar lining fluid, resulting in a high local plasma concentration, which is beneficial in treating the pulmonary disease.

In some embodiments, the amount of API particles dispensed in a single metered does which have a particle diameter of less than about 1.1 μm or less is at least about 25.0%, about 27.5%, about 30.0%, about 32.5%, about 35.0%, about 37.5%, about 40.0%, about 42.5%, about 45.0%, about 47.5%, about 50.0%, about 52.5%, about 55.0%, about 57.5%, about 60.0%, about 65.0%, about 70.0%, about 75.0%, about 80.0%, about 85.0%, about 90.0%, about 95.0%, or more.

In some embodiments, nozzle 508 is configured to release a spray of the API particles for a certain distance or a “jet length.” As used herein, the term “jet length” may convey that the inhalation pharmaceutical formulation “jets” out of the distal end of the nozzle as an aerosol spray.

In some embodiments, nozzle 508 has a jet length in a range of 0.5 mm to 1.0 mm, including subranges, for example 0.6 mm to 0.9 mm and 0.7 mm to 0.8 mm. In some embodiments, the jet length is about 0.5 mm, about 0.6 mm, about 0.7 mm, about 0.8 mm, about 0.9 mm, or about 1.0 mm.

In some embodiments, the MDI actuator 500 includes a stem block, for example stem block 509, shown in FIG. 7B, In some embodiments, stem block has 509 an inner diameter in a range of 2.5 mm to 4.0 mm, including subranges, such as 2.6 mm to 3.9 mm, 2.7 mm to 3.8 mm, 2.8 mm to 3.7 mm, 2.9 mm to 3.6 mm, 3.0 mm to 3.5 mm, 3.1 mm to 3.5 mm, 3.1 mm to 3.4 mm, or 3.2 mm to 3.3 mm. In some embodiments, stem block 509 has an inner diameter of about 2.5 mm, about 2.6 mm, about 2.7 mm, about 2.78 mm, about 2.8 mm, about 2.9 mm, about 3.0 mm, about 3.1 mm, about 3.16 mm, about 3.2 mm, about 3.3 mm, about 3.4 mm, about 3.5 mm, about 3.6 mm, about 3.7 mm, about 3.8 mm, about 3.9 mm, or about 4.0 mm.

In some embodiments, stem block 509 is tapered outward towards its proximal end, and has an inner diameter toward its distal end in a range of 3.0 mm to 4.0 mm, including subranges, such as 3.1 mm to 3.9 mm, 3.1 mm to 3.5 mm, 3.2 mm to 3.8 mm, 3.3 mm to 3.7 mm, or 3.4 mm to 3.6 mm. In some embodiments, the tapered stem block 509 has an inner diameter of about 3.0 mm, about 3.1 mm, about 3.16 mm, about 3.2 mm, about 3.3 mm, about 3.4 mm, about 3.5 mm, about 3.6 mm, about 3.7 mm, about 3.8 mm, about 3.9 mm, or about 4.0 mm.

In some embodiments, MDI actuator 500 is configured to provide a sump volume of 5.0 μL to 45.0 μL, including subranges, for example 5.0 μL to 30.0 μL, 10.0 μL to 25.0 μL, or 15.0 μL to 20 μL. In some embodiments, MDI actuator 500 is configured to provide a sump volume of about 5.0 μL, about 6.0 μL, about 7.0 μL, about 8.0 μL, about 9.0 μL, about 9.6 μL, about 10.0 μL, about 10.3 μL, about 11.0 μL, about 11.9 μL, about 12.0 μL, about 12.7 μL, about 13.0 μL, about 14.0 μL, about 15.0 μL, about 16.0 μL, about 17.0 μL, about 18.0 μL, about 19.0 μL, about 20.0 μL, about 25.0 μL, about 30.0 μL, about 35.0 μL, about 40.0 μL, about 40.7 μL, or about 45.0 μL.

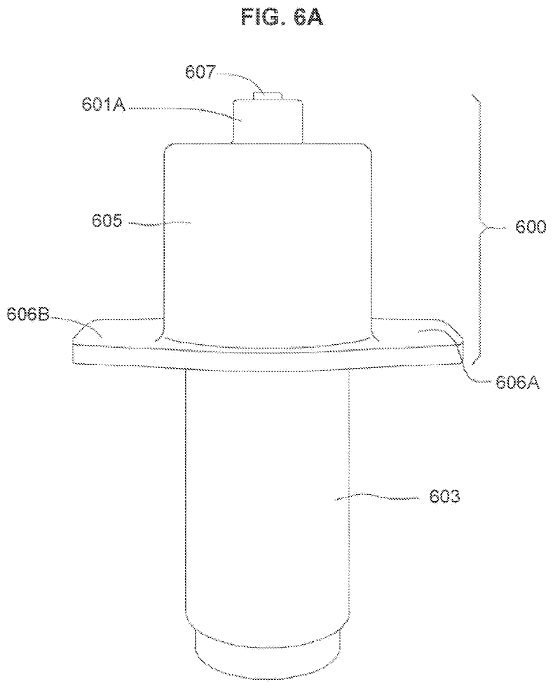

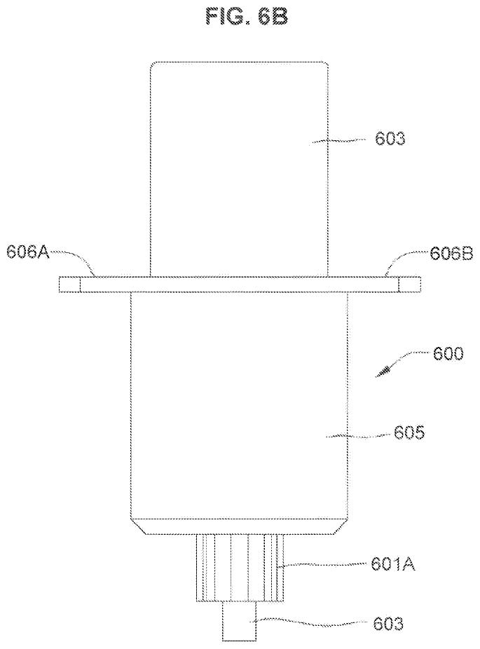

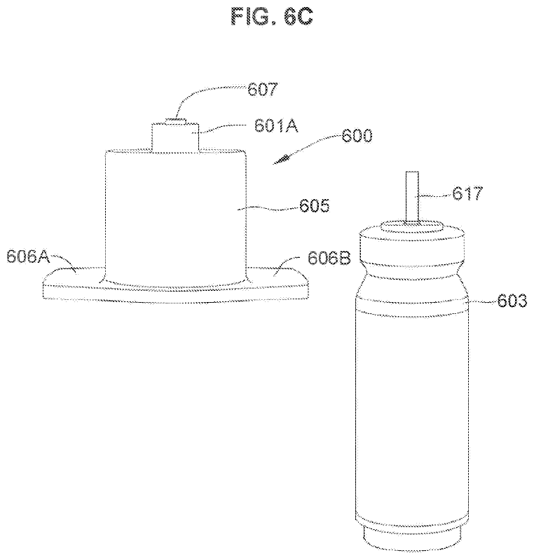

In some embodiments, MDI actuator 500 includes an insert 507, for example as shown in FIGS. 6A-6C. Insert 507 may have an outer diameter in a range of 4.0 mm to 5.0 mm, including subranges, such as 4.0 mm to 4.5 mm, 4.1 mm to 4.9 mm, 4.2 mm to 4.8 mm, 4.3 mm to 4.7 mm, or 4.4 mm to 4.6 mm. In some, the insert has an outer diameter of about 4.0 mm, about 4.1 mm, about 4.2 mm, about 4.3 mm, about 4.4 mm, about 4.5 mm, about 4.6 mm, about 4.7 mm, about 4.8 mm, about 4.9 mm, or about 5.0 mm. In some embodiments, insert 507 is tapered inward towards its distal end. In some embodiments, the insert has an outer diameter that adheres to an ISO standard, namely ISO 80369-7 2016, and thus, is about 4.4 mm and tapered inward, at a slope of about 3.44° or about 6%, towards its distal end.

In some embodiments, insert 507 has an inner diameter in the range of 0.5 mm to 2.5 mm, including subranges, such as from 0.6 mm to 2.4 mm, 0.7 mm to 2.3 mm, 0.8 mm to 2.2 mm, 0.9 mm to 2.1 mm, 1.0 mm to 2.0 mm, 1.1 mm to 1.9 mm, 1.2 mm to 1.8 mm, 1.3 mm to 1.7 mm, or 1.4 mm to 1.6 mm. In some embodiments, insert 507 has an inner diameter of about 1.0 mm or about 2.0 mm.

In some embodiments, MDI actuator 500 includes a crown having a one or more configurations. The cone configuration be: (i) flat; (ii) a ϕ1.6 plus 90° cone; (iii) a ϕ1 plus 90° cone plus ϕ3; (iv) a ϕ2.78 sphere; or (v) a ϕ3.18 sphere. In some embodiments, the cone angle can be in a range of 60° to 120°, including subranges such as 65° to 115°, 70° to 110°, 75° to 105°, 80° to 95°, 80° to 100°, or 85° to 95°. In some embodiments, the crown has a cone angle of about 60°, about 65°, about 70°, about 75°, about 80°, about 85°, about 90°, about 95°, about 100°, about 110°, or about 120°.

In some embodiments, the crown has a depth in a range of 0.4 mm to 3.0 mm, including subranges, for example 0.4 mm to 0.7 mm, 0.4 mm to 0.6 mm, 0.5 mm to 2.9 mm, 0.6 mm to 2.8 mm, 0.7 mm to 2.7 mm, 0.8 mm to 2.6 mm, 0.9 mm to 2.5 mm, 1.0 mm to 2.4 mm, 1.1 mm to 2.3 mm, 1.2 mm to 2.2 mm, 1.3 mm to 2.1 mm, 1.4 mm to 2.0 mm, 1.5 mm to 1.9 mm, or 1.6 mm to 1.8 mm. In some embodiments, the crown depth is about 0.5 mm, about 0.55 mm, about 0.6 mm, about 0.65 mm, about 0.7 mm, about 0.75 mm, about 0.80 mm, about 0.85 mm, about 0.90 mm, about 0.95 mm, about 1.0 mm, about 1.25 mm, about 1.50 mm, about 1.75 mm, about 2.00 mm, about 2.25 mm, about 2.50 mm, about 2.75 mm, or about 3.0 mm.

In some embodiments, actuator 500 further includes at least one handle support, for example handle supports 506A and 506B, shown in FIGS. 6A-6C, where the at least one handle support 506A is configure to engage with at least one finger of a patient to actuate the pharmaceutical formulation from the MDI. In some embodiments, actuator 500 includes at least two handle supports 506A and 506B,

To use the actuator 500 to dispense an API, for example HCQ, canister 524 may be pushed down, for example by a finger, into actuator 500 towards the distal end of the MDI, while another finger can engage the distal end of the MDI actuator by pushing upward to in order to administer the pharmaceutical formulation from canister 524 into the patient's throat such that it may travel through the respiratory tract into the patient's lungs.

In some embodiments, actuator 500 includes a connector fitting, for example Luer-lock fitting 501A, shown for example in FIGS. 6A-6C. In some embodiments, actuator 500, including connector fitting 501A and insert 507, is made as a one-piece assembly. However, in some embodiments, actuator 500, connector fitting 501A, and insert 507 are individual components that are configured to be assembled together. Additionally, in some embodiments, insert 507 is made as a one-piece assembly while actuator 500 and connector fitting 501A are made as a second one-piece assembly such that both insert 507 and the assembly of actuator 501 and connector fitting 501A may be operatively connected together.

In some embodiments, actuator 500, including connector fitting 501A and insert 507, is made of one of Delrin® material, polypropylene, polycarbonate, acrylonitrile butadiene styrene (“ABS”), or other suitable materials, or any combination thereof.

In some embodiments, actuator 500, including the nozzle, is made of, or made substantially of, polyoxymethylene (“POM”), polypropylene (“PP”), polycarbonate (“PC”), acrylonitrile butadiene styrene (“ABS”), high-density polyethylene (“HDPE”), or other suitable materials. In other embodiment, actuator 500 can be made of, or made substantially of, clear or transparent PC, or other suitable materials to enable viewing of an add-on dose-counter.

Targeted-Delivery MDI Having an Airtight Connector Fitting for Use with Auxiliary Delivery Components

As discussed above, patients with severe COVID-19, or other pulmonary viral diseases, are often placed on ventilators to assist with the patient's difficulty breathing or his or her inability to breathe. For example, more than 40% of infected COVID-19 patients develop acute respiratory distress syndrome (“ARDS”), a condition with a high mortality rate, or other serious respiratory ailments. ARDS often causes a buildup of fluid within the alveoli, which severely impairs breathing. As the gas transfer process within the lungs is impaired and oxygen levels fall, ventilators work to keep patients breathing.

In order for the ventilator to transport and exchange air, oxygen, and carbon dioxide to and from the patient's lungs, various ventilator connectors, such as ventilator tubing, are provided to connect the ventilator to the patient's mouth, leading into the trachea. Alternatively, the ventilator connector may lead directly into a patient's trachea via tracheostomy (e.g. a surgically made hole that goes through the front of a patient's neck and into the trachea), thus obviating the need to enter the trachea through the mouth. In either situation, the ventilator circuitry commonly includes several ventilator connectors operatively connected to the ventilator and the patient.

As used herein, the phrase “operatively connected” to a ventilator means that a ventilator is connected directly (e.g. in direct contact) or indirectly (e.g. through one or more ventilator circuitry having one or more ventilator connectors or ventilator tubing) to a patient, and through this connection, the ventilator may provide air exchange with the patient. A patient may be indirectly connected to the ventilator via a ventilator circuitry having one or more ventilator connectors, such as ventilator tubing. The ventilator circuitry may also include a humidifier and a water trip, which may be operatively connected to the patient and the ventilator through one or more ventilator connectors, such as ventilator tubing.

MDI's, for example the MDI's discussed above, may be used in conjunction with ventilators to deliver certain medications, for example HCQ. This method of drug delivery may provide an advantage over certain aerosol treatment procedures for COVID-19, which may be effective, but may also cause CoV2 to be release from the patient into the ambient air, thereby putting health care professionals at a greater risk for contracting COVID-19.

Unfortunately, the currently available ventilator adapters for MDI delivery are unable to effectively deliver certain inhalation pharmaceutical formulations. As background, inhalation pharmaceutical formulations are typically housed in a pressurized canister and are administered as a metered dose per actuation using a handheld aerosol MDI actuator device, for example actuator 500 described above. However, when a patient is on a ventilator, self-administrating the MDI is not practical. Instead, the inhalation pharmaceutical formulation, in aerosolized or nebulized form, is administered into the ventilator circuitry to ensure that the formulation travels properly through the patient's respiratory tract.

The current ventilator circuitry adapters do not provide a stable, secure connection between a ventilator connector and the MDI actuator. In particular, the current ventilator circuitry adapters rely primarily on the friction created between a stem of an MDI canister and the adapter cavity to create the “connection.” As a result, this “connection” is not stable, which is not practical when multiple MDI actuations (or sprays) are needed to administer the therapeutically effective dose. For instance, when multiple MDI actuations are needed, a pause of about 45 seconds to 1 minute between MDI actuations is typically needed in order for the patient to have time to sufficiently inhale each MDI actuation. Additionally, when the “connection” is not stable, it is difficult to accurately and consistently dispense the formulation. For instance, if the “connection” is angled, then the formulation may be dispensed towards the sides of the ventilator connector, thereby compromising the treatment and wasting valuable formulation.

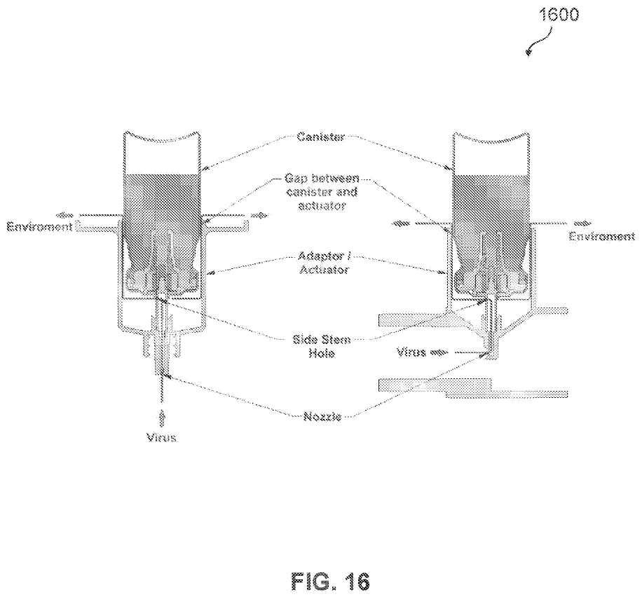

Moreover, due to the unavoidable “side stem hole” of a typical MDI structure, a possible “leak path” is created for the virus-contaminated air in the ventilator circuit to escape to the ambient environment. For example, the air exhaled from the patients is allowed to flow freely inside the ventilator circuit. However, it is noted that currently in MDI valve stems, there is a hole, having an diameter of about 0.5 mm, called “side stem hole” or “transfer hole,” which plays the role of transferring medication to the stem for dosing. This hole, which may have a diameter of about 100 nm, may allow for leakage of air that may be contaminated by CoV2 when it is at rest position. This is because CoV2 has a size of approximately 100 nm (60-120 nm), making it small enough to fit through the side stem hole. This “leak path” issue is outlined in diagram 1600, shown for example in FIG. 16. ISO standard 5367 dictates the design of anaesthetic and respiratory equipment breathing sets and connectors. One requirement of ISO 5367 is that, with respect to leakage from a complete breathing set or breathing tube supplied for use with a ventilator breathing system, the leakage should not exceed the limit of 70 ml/min for and adult at 60 hPa. However, with even a small leakage hole having a diameter of 0.2 mm or larger, the system ends up with a leakage of at least 130 ml/min at 60 hPa.

Furthermore, in order to help seriously ill patients who rely on a ventilator to breathe, it may be necessary to administer an API having a particle size of less than 5 μm in order to ensure the API reaches the entire upper and lower airway, including the lungs. Further, API particles that target to deep, peripheral lungs, alveoli, or alveoli lining fluid may have a particle size of less than 2 μm to enable effective treatment of diseases that cause infections and lesions in deep, peripheral lungs. Unfortunately, the MDI adaptors that are currently available may not be able to control the particle size distribution of the API's having such a small particle size. In fact, current MDI assemblies do not possess the functionality necessary to control particle size distribution, nor do they include air tight, leak proof, or virus mitigating features.

Additionally, the current ventilator circuitry adapters do not have sufficient guides to align the canister of the MDI to the center of the MDI actuator in order to accurately and consistently dispense the formulation towards the patient. Similar to the problem presented by a weak “connection,” described above, if the canister is not properly aligned, the formulation may be dispensed towards the sides of the ventilator connector, thereby compromising the treatment and wasting valuable formulation. Accordingly, this problem may be compounded when multiple MDI actuations are needed to arrive at the therapeutically effective dose.

Finally, current ventilator circuitry adapters do not have handles for easy and reliable dispensing, which can result in similar problems to those described above. Moreover, when all of these problems are combined, the synergic disadvantages are exasperated.

Because of these key technical limitations and disadvantages with the currently available devices and connectors in a ventilator circuitry for MDI administration, there is an unmet need for MDI actuators which can more efficiently and safely deliver inhalation pharmaceutical formulations to patients operatively connected to ventilators, and which may be designed to have leak-proof and virus mitigating features to help protect healthcare providers and meet ISO standard 5367.

Accordingly, described herein are embodiments of an airtight ventilator device designed to protect healthcare professionals from leakage of contagious air exhaled by mechanically ventilated patients during a course of treatment of MDI medications. The ventilator device is easy to use, which allows for quick, reliable, and effective administration of aerosolized medication.

In some embodiments, the ventilator device is configured to both act as an MDI actuator and an adaptor to connect an MDI to a ventilator circuit. The ventilator device may mitigate the transfer of viruses when delivering a medication into a ventilator circuit from an MDI by providing an airtight connection to the ventilator circuit and a leak-proof seal between the device and the MDI canister. These features may mitigate the risk of aerosolization of contaminated air from the ventilator circuit, such as a virus exhaled by a patient, from escaping to the ambient environment. In this way, the healthcare providers who work around the patients may be provided with protection from infection by an aerosolized virus, for example CoV2.

In some embodiments, the disclosed MDI's and methods produce aerosolized product particles that have a size distribution within a small range, including but not limited to fine drug particles (e.g., less than 4.7 μm particle diameter), and extra-fine drug particles (e.g., less than 1.1 μm particle diameter). Advantageously, this particle size control may enable the delivery of the drug to various targeted areas of the respiratory tract, for example the deep, peripheral lungs, alveoli, or alveoli lining fluid.

In some embodiments, the MDI actuators include a housing with a cylindrical “cup” for containing an MDI or an MDI with an add-on dose counter and two finger grips to be hand-held by a user, which may enable the user to use commercially available MDI units with or without add-on dose counters on mechanically ventilated patients.

In some embodiments, the MDI actuators configured for use with auxiliary delivery components that are disclosed herein are configured to provide ventilator-delivery of pharmaceutical formulations to a patient operatively connected to a ventilator via a connector fitting for connecting to a corresponding connector fitting of a ventilator connector, such as ventilator tubing. The ventilator connector may be capable of operatively connecting to both a patient and a ventilator via one or more ventilator circuitry components. In some embodiments, the connector fitting is a Luer-lock fitting configured to connect to a corresponding Luer-lock fitting of the ventilator connector. The Luer-lock fitting may provide a stable connection between the MDI actuator and the ventilator connector to enable an efficient and effective dispense, per actuation, of the formulation.

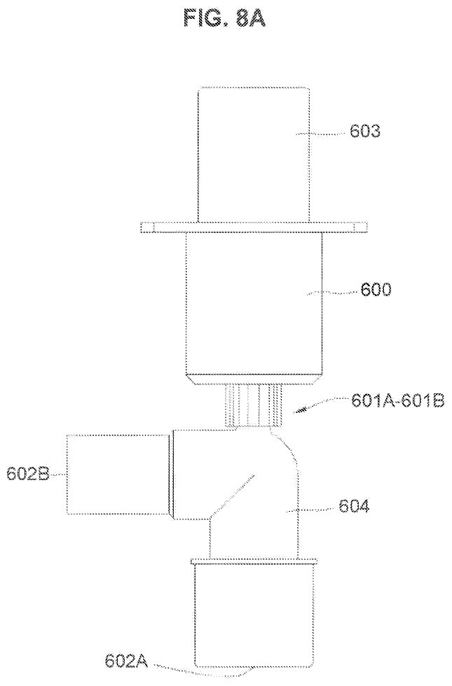

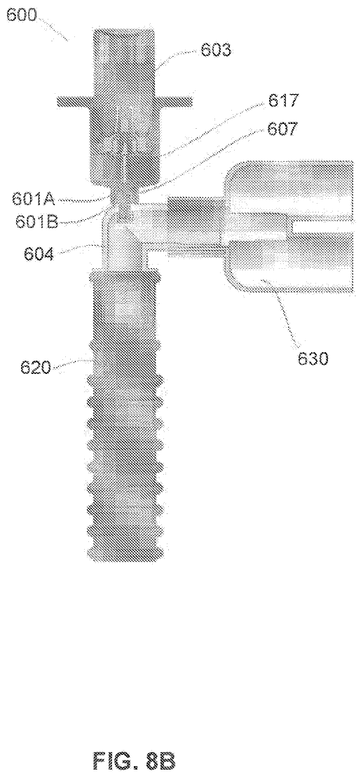

For example, as shown in FIG. 8A, MDI actuator 600 is configured to dispense, per actuation, inhalation pharmaceutical formulation from a MDI 603, such as a MDI canister, and into a ventilator connector, such as ventilator tubing, that is operatively connected to a ventilator and a patient via a ventilator circuitry. The disclosed MDI's may allow for a closed ventilator circuitry system to be maintained without disruption during administration of one or more metered doses of a pharmaceutical formulation.

Additionally, as shown in FIG. 8B, for example, MDI actuator 600 can be connected to a connector 604, which may have an elbow configuration, through a port 601B. Connector 604 may alternatively be one of a ventilator connector having a configuration other than an elbow configuration, an adaptor, tubing, a component, and the like. Connector 604 may be in the main ventilator circuit and may be used to connect a corrugated tube 620 and a Y-branch 630. Corrugated tube 620 may connect to an endotracheal tube for the patient, while Y-branch 630 may connect to inspiratory and expiratory tubes of a ventilator. The air exhaled from a patient may flow freely through the corrugated tube 620, connector 604, Y-branch 630, and to an expiratory tube inside the ventilator circuit.

In some embodiments, actuator 600 includes an insert, for example insert 607, shown in FIGS. 6A-6C, and a connector fitting 601A for connecting to a corresponding fitting of a ventilator connector. In particular, connector fitting 601A may be a Luer-lock fitting. In some embodiments, MDI actuator 600 is suitable for use with MDI 603, which includes a pressurized aluminum canister having inhalation pharmaceutical formulation disposed therein, and is capable of dispensing, per actuation or spray, a metered dose of the inhalation pharmaceutical formulation. MDI 603 may be actuated by MDI actuator 600.

Connector fitting 601A may be an industry standard Lueur-lock fitting, which may connect with a corresponding Luer-lock fitting on a ventilator connector, such as ventilator connector 604. Connector fitting 601A may connect with a corresponding Luer-lock fitting 601B by rotation with respect to one another. This connection may help stabilize the path of the inhalation pharmaceutical formulation into ventilator connector 604, which may facilitate drug delivery of the pharmaceutical formulation through the patient's respiratory tract into his or her lungs and alveoli.

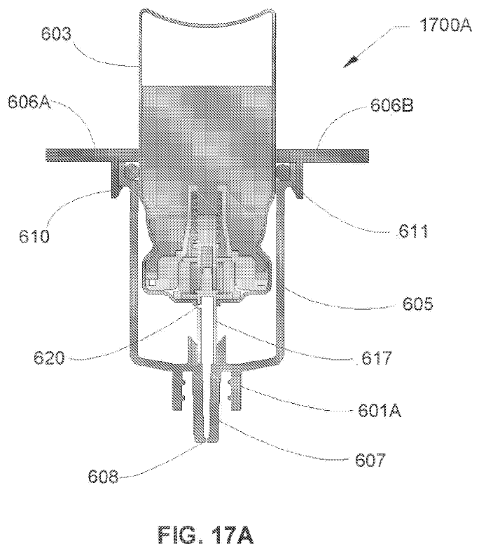

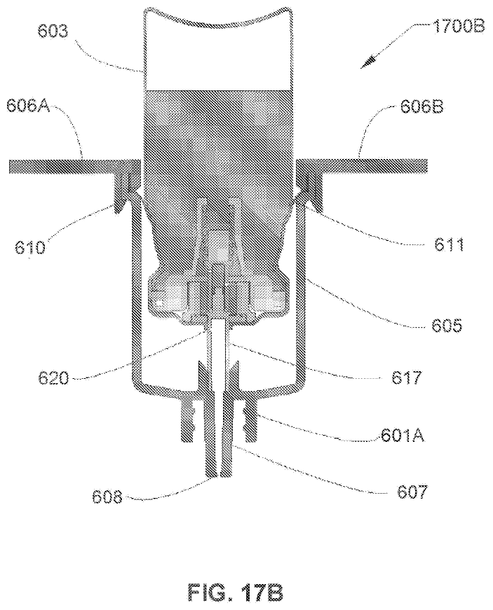

In some embodiments, for example as shown in FIGS. 17A and 17B, MDI actuator 600 includes a nozzle 608. Nozzle 608 may have a diameter in the range of 0.15 mm to 0.25 mm, including subranges, such as 0.15 mm to 0.20 mm, 0.18 mm to 0.22 mm, and 0.20 mm to 0.25 mm. In some embodiments, nozzle 608 has a diameter of about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, or 0.25 mm.

In some embodiments, nozzle 608 has a jet length in a range of 0.3 mm to 1.0 mm, including subranges, such as from 0.3 mm to 0.9 mm, 0.3 mm to 0.6 mm, 0.4 mm to 0.9 mm, 0.5 mm to 0.8 mm, 0.6 mm to 1.0 mm, or 0.6 mm to 0.7 mm. In some embodiments, nozzle 508 has a jet length of about 0.3 mm, about 0.5 mm, about 0.7 mm, and about 1.0 mm. In a preferred embodiment, the jet length is about 0.7 mm.

In some embodiments, towards the distal end of MDI actuator 600, insert 607 has a longer length than the length of connector 601A, which may have a standard length in order to cooperate with a corresponding Luer-lock connector on a ventilator circuit connector or adaptor on the ventilator circuit, such as an elbow adaptor. The longer length of insert 607 may aid in reducing or preventing aerosolized inhalation pharmaceutical formulation from sticking to the sides of the ventilator circuit, thereby improving delivery and treatment effectiveness. In some embodiments, the insert length is in a range of 10.0 mm to 22.0 mm, including subranges, such as from 11.0 mm to 21.0 mm, 12.0 mm to 20.0 mm, 13.0 mm to 19.0 mm, 14.0 mm to 18.0 mmm, 15.0 mm to 17.0 mm, or 15.0 mm to 19.0 mm. In some embodiments, the insert length is about 10.0 mm, about 11.0 mm, about 12.0 mm, about 13.0 mm, about 14.0 mm, about 15.0 mm, about 16.0 mm, about 17.0 mm, about 18.0 mm, about 19.0 mm, about 20.0 mm, about 21.0 mm, and about 22.0 mm.

Further, the insert 607 can have an inner diameter corresponding to the sump depth. Accordingly, in some embodiments an inner diameter of insert 607 is in a range of 0.5 mm to 2.5 mm, including subranges, such as 0.6 mm to 2.4 mm, 0.7 mm to 2.3 mm, 0.8 mm to 2.2 mm, 0.9 mm to 2.1 mm, 1.0 mm to 2.0 mm, 1.1 mm to 1.9 mm, 1.2 mm to 1.8 mm, 1.3 mm to 1.7 mm, or 1.4 mm to 1.6 mm. In some embodiments, insert 507 has an inner diameter of about 1.0 mm or about 2.0 mm, and an outer diameter of 4.0 mm to 5.0 mm, such as about 4.4 mm. The sump depth and corresponding inner diameter provides the sump volume. Thus, in some embodiments, MDI actuator 600 is configured to provide a sump volume in a range of 5.0 μL to 45.0 μL, as will be discussed below. In some embodiments, canister stem 617 provides the valve stem bore internal volume or “stem volume.”

With reference to FIGS. 7A-7D, MDI actuator 600 may include a connector fitting 601A, body 605, two support handles 606A and 606B, and insert 607. In particular, connector fitting 601A may be a Luer-lock fitting. A healthcare provider may hold support handles 606A and 606B and press down on the top of the MDI to actuate the medication into the ventilator circuit, synchronizing with inspiration, for direct medication delivery to the airway of a mechanically ventilated patient. This method of administration may be similar to that for parenteral injection.

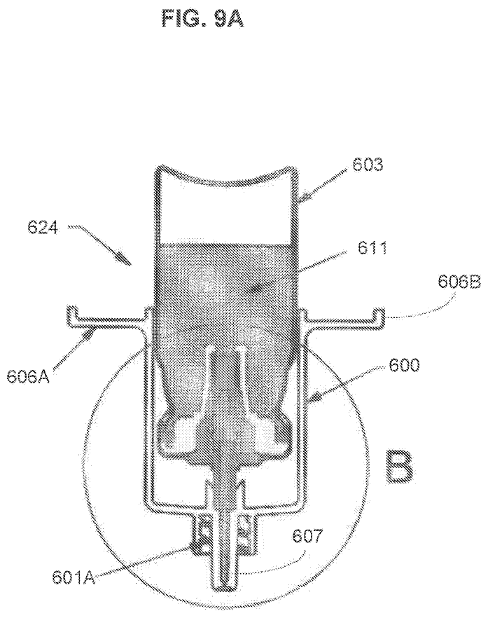

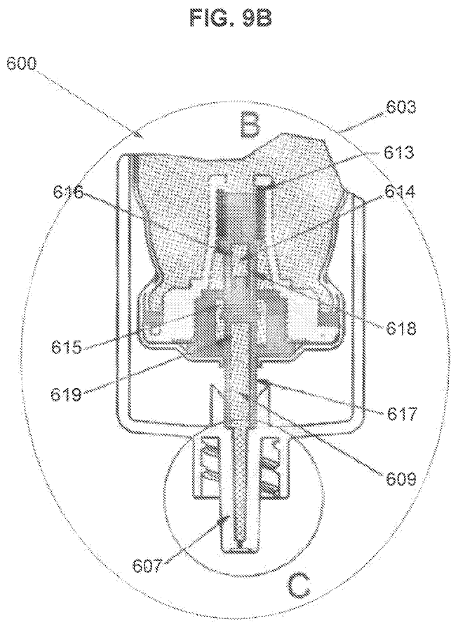

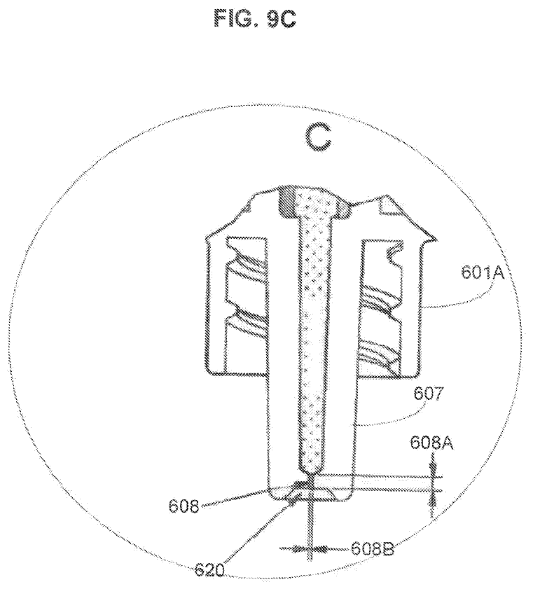

Body 605 may be configured to align a canister containing a pharmaceutical formulation, for example canister 624, shown in FIG. 9A. As shown in FIG. 7A, body 605 is substantially circular and may have an inner diameter. The inner diameter of body 605 may be about 22.0 mm to about 23.0 mm, and may correspond to an outer diameter of a MDI canister, such as canister 624.

Additionally, as shown in FIGS. 7A-7D, body 605 may be substantially hollow in order to accommodate and receive the canister. For example, FIGS. 7C and 7D show bottom and bottom perspective views, respectively, of MDI actuator 600. As shown, body 605 is substantially hollow and circular in order to accommodate, receive, align, and/or actuate the MDI canister to dispense the pharmaceutical formulation accurately.

Body 605 may not cover the entirety of the MDI canister, but may cover at least a portion of the MDI canister to allow for space between the canister and body 605, which may be necessary to enable the actuation of the pharmaceutical formulation from the canister when the canister is pushed downward toward the distal end of actuator 600.

As discussed above, in some embodiments, actuator 600 includes an insert 607. In some embodiments, towards the distal end of actuator 600, insert 607 has a longer length than the length of connector 601A, which may have a standard length in order to cooperate with a corresponding Luer-lock fitting on a ventilator connector. In some embodiments, insert 607 has a length in the range of 10.0 mm to 22.0 mm, including subranges, such as from 11.0 mm to 21.0 mm, 12.0 mm to 20.0 mm, 13.0 mm to 19.0 mm, 14.0 mm to 18.0 mm, or 15.0 mm to 17.0 mm. In some embodiments, insert 607 has a length of 12.0 mm, about 15.0 mm, about 17.0 mm, or about 20.0 mm, which may enable an efficient delivery of one or more actuations of the pharmaceutical formulation from the canister into the ventilator connector. Additional details regarding the dimensional relationships between insert 607 and connector 601A are shown, for example, in the circle (or identifier “C”) in FIG. 7A, and FIG. 7B, which is a zoomed-in view of the circle “C” shown in FIG. 7A.

As shown in FIG. 7D, for example, actuator 600 may include a tapered stem block 609. In some embodiments, stem block 609 has an inner diameter in a range of 3.0 mm to 4.0 mm towards its distal end, and may be and tapered outward towards its proximal end. The tapering of stem block 609 may allow for engagement of stem block 609 with the stem of the MDI canister in order to effectuate dispensing of the pharmaceutical formulation from the MDI canister.

In some embodiments, MDI actuator 600 is configured to provide a sump volume of 5.0 μL to 45.0 μL, including subranges, for example 5.0 μL to 30.0 μL, 10.0 μL to 25.0 μL, or 15.0 μL to 20 μL. In some embodiments, MDI actuator 500 is configured to provide a sump volume of about 5.0 μL, about 6.0 μL, about 7.0 μL, about 8.0 μL, about 9.0 μL, about 9.6 μL, about 10.0 μL, about 10.3 μL, about 11.0 μL, about 11.9 μL, about 12.0 μL, about 12.7 μL, about 13.0 μL, about 14.0 μL, about 15.0 μL, about 16.0 μL, about 17.0 μL, about 18.0 μLL, about 19.0 μL, about 20.0 μL, about 25.0 μL, about 30.0 μL, about 35.0 μL, about 40.0 μL, about 40.7 μL, or about 45.0 μL.

FIGS. 9A-9C show a detailed view of MDI actuator 600, which includes a canister 624, and an MDI 603, a connector 601A, a body 605, and support handles 606A and 606B. Connector 601A may be a Luer-lock connector. In some embodiments, actuator 600 includes an inhalation pharmaceutical formulation 611 disposed within a canister.

FIG. 9A shows MDI 603 in an actuated state, which is achieved when MDI 603 is pushed downward, for example using at least one finger, toward the distal end of actuator 621 while at least two other fingers push upward on support handles 606A-608B towards in the direction of the proximal end of actuator 600. In some embodiments, the distal end of actuator 600 is located towards insert 607, and the proximal end of actuator 600 is located towards the distal end of the canister of MDI 603.

In some embodiments, for example as shown in FIG. 9B, actuator 600 includes an insert 607, a sump 609 and a stem 617. Sump 609 may create a sump volume and stem 617 may create a stem volume. As will be described in Examples 4A-4I and Table 4, below, different configurations of the sump 609 and stem 617 can produce different sump volume values, for example sump volume values in a range of 5.0 μL to 45.0 μL, such as about 9.6 μL, about 10.3 μL, about 11.9 μL, about 12.7 μL, or about 40.7 μL.

In some embodiments, MDI 603 includes a compressed spring 613, a buffer 614, and a metered dose 615. MDI 603 may additionally include various passways, which may allow for an inhalation pharmaceutical formulation to travel from the canister of MDI 603 to actuator 600. In some embodiments, first passway 616 may allow for distribution of an inhalation pharmaceutical formulation from the canister of MDI 603 to buffer 614, while second passway 618 may allow for distribution of an inhalation pharmaceutical formulation from buffer 614 to metered dose 615. In some embodiments, metered dose 615 is capable of dispensing a metered dose, per actuation, of the inhalation pharmaceutical formulation, and third passway 619 may allow for distribution of the inhalation pharmaceutical formulation from metered dose 615 to sump 609.

In some embodiments, for example as shown in FIG. 9C, insert 607 includes a nozzle 608 and a nozzle crown 620. Nozzle 608 may have an inner diameter 608B and a jet length 608A. In some embodiments, an inner diameter 608B of jet length 608A may have be constant from the proximal end to the distal end of jet length 608A.

In some embodiments, the crown 620 has one of a flat configuration, a ϕ1.6 plus 90° cone configuration, a ϕ1 plus 90° cone plus ϕ3 configuration, a ϕ2.78 sphere configuration, or a ϕ3.18 sphere configuration.

### Controlling Leakage in MDI Actuator Assemblies Including Ventilators

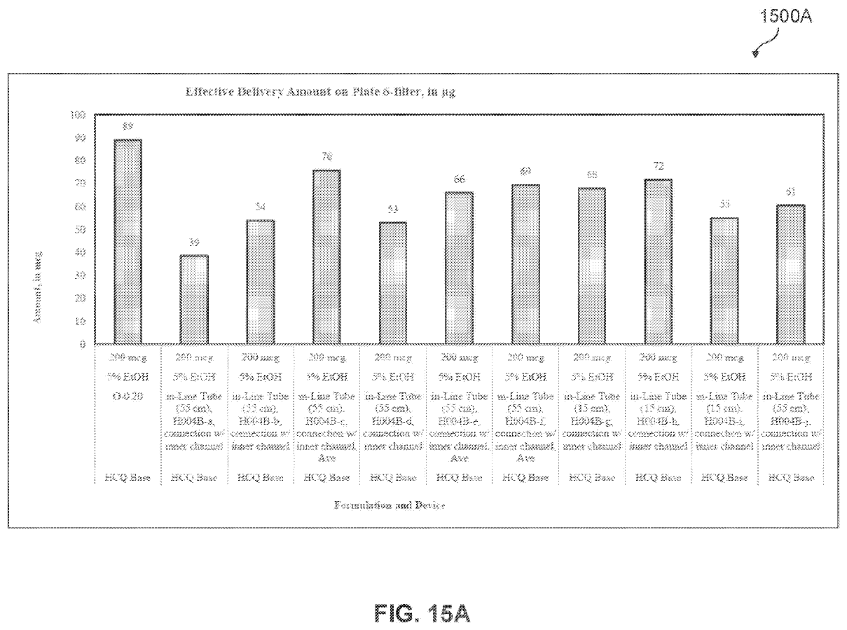

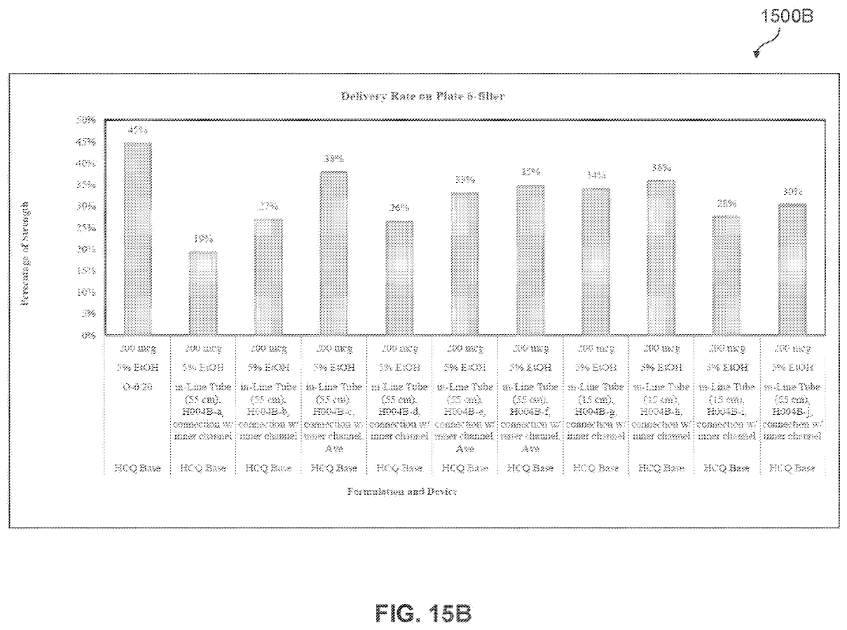

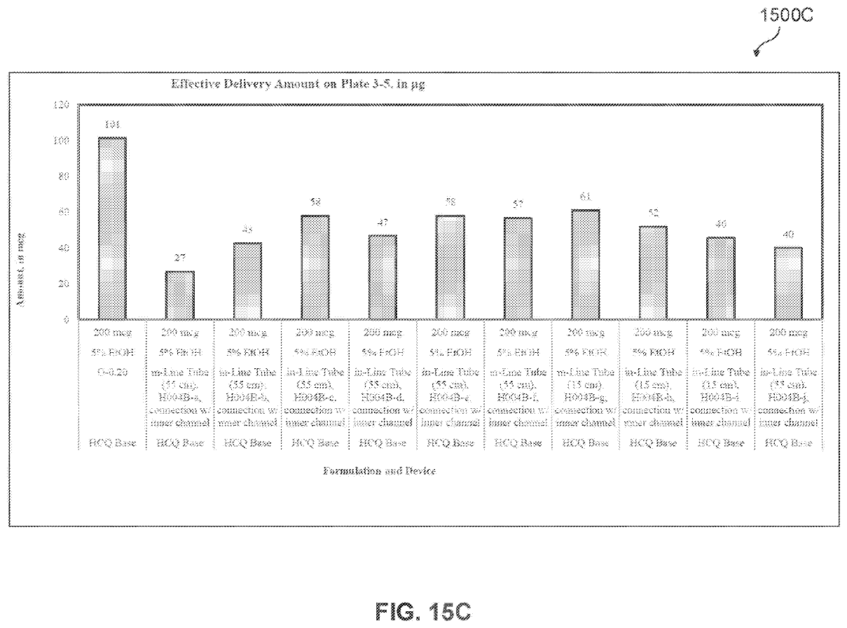

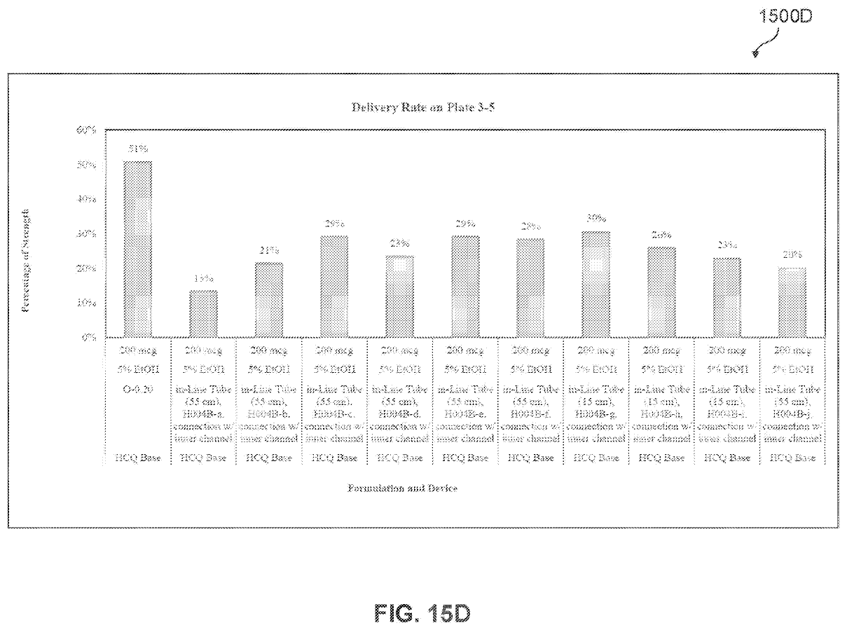

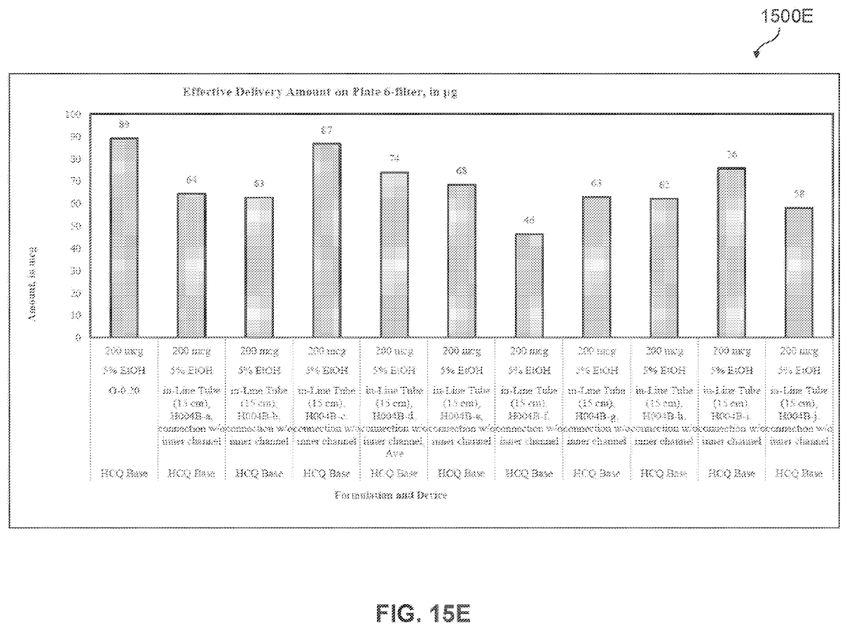

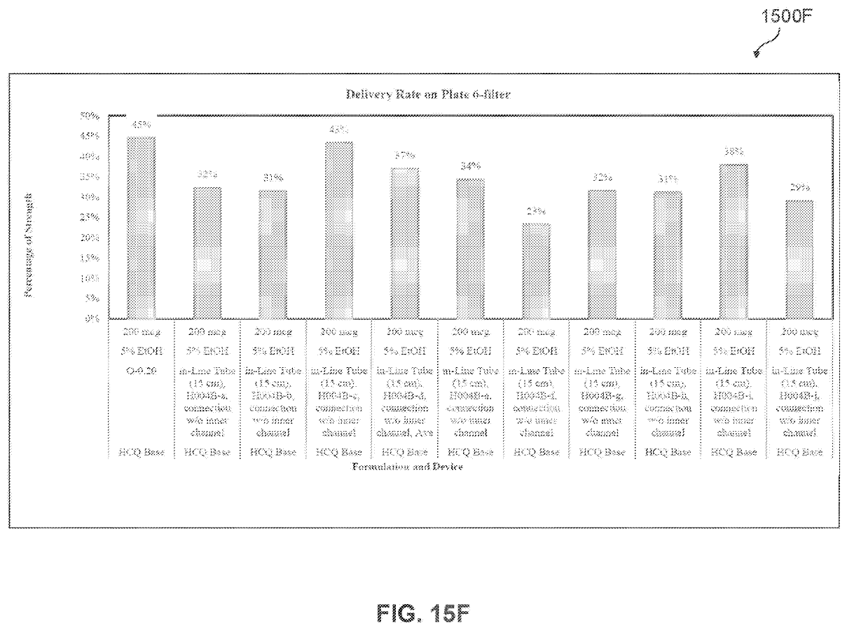

As discussed above, in the valve stem of many different MDI's, there may be a small hole called “side stem hole” or “transfer hole,” which may facilitate the transfer of medication to the stem for dosing. As shown in FIG. 15, for example, the moment a canister is pressed, side stem hole may retract into the metered dose chamber. At this point, the side stem hole may become a pathway which allows a drug formulation to flow out of the metered dose chamber to the stem for dosing.

As shown in FIG. 16, at rest position, the side stem hole may allow for leakage of air contaminated by virus, through a leak path, from the ventilator circuit, to the actuator nozzle, to the transfer hole, to the gap between the device and MDI canister, and finally to the ambient environment. This leakage may result in a risk of virus transmission to health care professionals.

Due to the transfer hole, the gap between the device and the MDI may create a path for potential leakage, resulting in non-compliance with ISO 5367 standard, as discussed above. To assess the impact of the described leakage from the “transfer hole”, 9 different currently commercially available metered-dose canisters were measured, and each had a transfer hole diameter in the range of 0.45 mm to 0.65 mm.

To calculate the air flow rate through a nozzle to assess leakage rate from the transfer hole, Bernoulli's equation (Equation 1, below) was used.

$${Q}_{a} = \frac{1}{60} \cdot 4.17 \cdot C \cdot {( \frac{{d}_{0}}{4.654} )}^{2} \cdot {p}_{1} \cdot ( 1 - \frac{\frac{{p}_{1} - {p}_{2}}{{p}_{1}}}{3 {F}_{γ} \cdot {x}_{T}} ) \cdot \sqrt{\frac{\frac{{p}_{1} - {p}_{2}}{{p}_{1}}}{{T}_{a} + 273.15}} . \qquad ( 1 )$$

### Where,

Tₐ: Air Temperature (° C.)
p₁: Primary Pressure (kPa abs)
p₂: Secondary Pressure (kPa abs)
d₀: Diameter of Nozzle (mm)
C: Discharge Coefficient (=0.7 for general non sharp edge nozzle)
dₐ: Air Flow Rate (Normal) (Nm³/min)
F_{γ}: Specific heat ratio factor (=Specific heat ratio/1.4)
x_{T}: Pressure differential ratio factor (=0.72)
   At 60 hPa and at 20° C., it can be shown from Eq (1) that for a “transfer hole” of 0.45 mm diameter, a leakage rate of 657 mL/min may obtained. Similarly, for a transfer hold having a 0.2 mm diameter, the leakage rate is 130 mL/min. Both theoretically calculated leakage rates indicate that a significant amount of contaminated air exhaled from a patient may escape from the gap to the ambient environment.

To block the gap between the device and the MDI canister, as shown in FIG. 17A, some embodiments of a ventilator device 1700A include elastic ring 611A, which may be an O-ring, X-ring, or any other elastic ring, may be implemented between canister and internal diameter of the actuator. Leak tests were conducted for devices having O-rings made from five different materials, including Silicone, Viton, Buna-N, Neoprene, and EPDM. The results showed that the leakage values for these devices were in the range of 3×10−7 to 1.2×10−6 l/min at 1 bar, which corresponds to a the leakage rate of a single, 140 nm to 90 nm-sized hole. Such a leakage rate is negligible compared to the ISO 5367 requirement of 70 mL/min at 60 hPa.

In some embodiments, for example as shown in FIG. 17B, instead of an elastic ring, an elastic film 611B, is used. Elastic 611B may provide a lower resistance between the canister and ring when sliding.

FIG. 17A shows a first embodiment of a ventilator device 1700A. As shown in FIG. 17A, connector 601A, which may be a Luer-lock connector, creates a leak proof connection when connected to a mating ventilator circuit port, thereby mitigating the risk of aerosolization of a virus. As discussed above, a virus may travel through nozzle 608 to the sump, inside of stem 607, through side stem hole 620, then to a gap between MDI 603 and actuator 600, and finally escape out of the ventilator circuit. To block this pathway, elastic ring 611A may be implemented between canister and internal diameter of the actuator.

FIG. 17B shows another embodiment of a ventilator device 1700B. In contrast to the embodiment shown in FIG. 17A, an elastic film 611B, is implemented rather than elastic ring 611A. Elastic film 611B may provide for lower resistance between MDI 603 and the actuator 600 during use.

In some embodiments, elastic ring 611A is made of at least one of silicone rubber (SiR), nitrile rubber (NBR, Buna-N), ethylene propylene diene monomer (EPDM), ethylene propylene rubber (EPR), polychloroprene (neoprene), polytetrafluoroethylene (PTFE), Polyisoprene (IR), butyl rubber (IIR), polyacrylate rubber (ACM), butadiene rubber (BR), sanifluor (FEPM), fluoroelastomer (FKM), fluoroelastomer (FKM), perfluoroelastomer (FFKM), polysulfide rubber (PSR), styrene-butadiene rubber (SBR), chlorosulfonated polyethylene (CSM), or blends thereof.

An example of a ventilator device, for example ventilator device 1700A, was tested and compared to a self-administrable, handheld MDI to determine the target site delivery efficiency in the ventilator circuit as compared to the MDI delivery efficiency without the ventilator circuit (e.g. no elbow connector or ventilator tubing). 200 mcg of HCQ formulation were administered in a single spray into an elbow connector, which was connected to a CI via 15 cm ventilator tubing. In this study, the HCQ formulation contained HCQ as the API, about 5% alcohol (EtOH), and about 95% propellant HFA-134a. As the control, 200 mcg of the HCQ formulation was also administered from the self-administrable, handheld MDI into the CI, without the elbow connector or 15 cm ventilator tubing. Delivery efficiency results are provided in Table 1, below. Advantageously, for the target particle size of 1.1 μm or less, as determined by EPM (6-filter) in Table 1, the ventilator device had a relatively high delivery efficiency of 34.8% compared to 44.5% of the self-administrable, handheld MDI. This result demonstrates that the target site delivery efficiency in the ventilator circuit is maintained over 78% of MDI delivery efficiency without the ventilator circuit.

TABLE 1
Ventilator Device Compared to Self-Administrable, Handheld MDI

| | | Self-Administrable | | In-Line MDI | |
|---|---|---|---|---|---|
| | | Handheld MDI | | elbow connection, | |
| | | (200 mcg) | | 15 cm tubing | |
| | | | Compared | | Compared |
| Items | | | to | | to |
| Stage # | Spec | Average | 200 mcg | Average | 200 mcg |
| Throat | | 16.2 | 8.1% | 2.2 | 1.1% |
| 0 | >9.0 | 0.7 | 0.4% | 0.5 | 0.3% |
| 1 | p1 5.8-9 | 1.0 | 0.5% | 0.6 | 0.3% |
| 2 | p2 4.7-5.8 | 1.9 | 1.0% | 1.1 | 0.5% |
| 3 | p3 3.3-4.7 | 10.3 | 5.2% | 4.4 | 2.2% |
| 4 | p4 2.1-3.3 | 32.7 | 16.3% | 15.7 | 7.9% |
| 5 | p5 1.1-2.1 | 58.4 | 29.2% | 41.2 | 20.6% |
| 6 | p6 0.65-1.1 | 26.4 | 13.2% | 19.8 | 9.9% |
| 7 | p7 0.43-0.65 | 8.4 | 4.2% | 7.2 | 3.6% |
| Filter | <0.43 | 54.2 | 27.1% | 42.7 | 21.3% |
| Total | [200 mcg] | 210.2 | 105.1% | 135.4 | 67.7% |
| % Recovery | 85~115% | 105% | NA | 67.7% | NA |
| Total 3~5 | NA | 101.3 | 50.7% | 61.3 | 30.6% |
| CPM (Throat-2) | | 19.9 | 9.9% | 4.5 | 2.2% |
| EPM (6-filter) | | 89.0 | 44.5% | 69.7 | 34.8% |
| ISM (0-filter) | | 194.0 | 97.0% | 133.2 | 66.6% |
| FPM (3-filter) | | 190.3 | 95.2% | 130.9 | 65.5% |

Examples of MDI Actuators for Use with an Auxiliary Delivery Component Having a Luer-Lock Fittings which Provides Highly Efficient Targeted Delivery of Inhalation Pharmaceutical Formulations

Examples 4A-4I, shown in Table 2, below, present non-limiting exemplary embodiments of MDI actuators, which may be configured for ventilator-delivery of inhalation pharmaceutical formulation to a patient having a pulmonary disease, for example COVID-19, who is operatively connected to a ventilator. In particular, each actuator shown in Examples 4A-4J may be configured for dispensing inhalation pharmaceutical formulation from a MDI container, such as a MDI canister, and into a ventilator connector, such as ventilator tubing, that is operatively connected to a ventilator and a patient. The MDI canister may be an aluminum canister having an inhalation pharmaceutical formulation, such as an HCQ pharmaceutical formulation, and may be capable of dispensing, per actuation (or spray), a metered dose of the API of the inhalation pharmaceutical formulation.

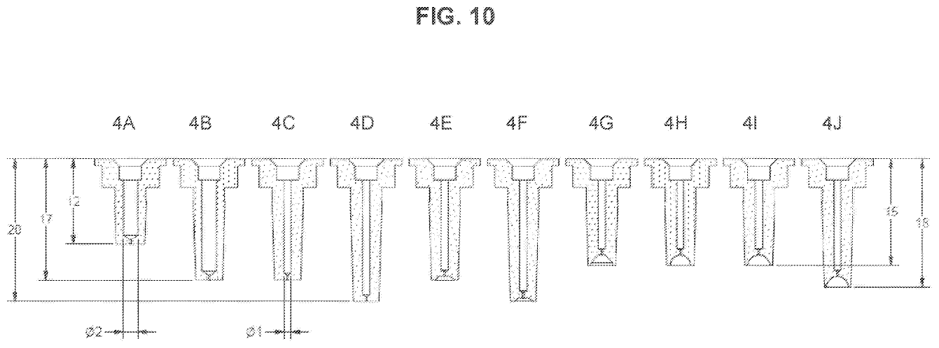

Each of the MDI actuators summarized in Table 2 were made substantially of Delrin® material. As shown in Table 2, each MDI actuator may have different sump and stem configurations that all may produce different sump volume minus stem volumes. The configurations of each of Examples 4A-4J are shown, for example, in FIG. 10.

TABLE 2
Exemplary Embodiments of MDI Actuators for Ventilator-Delivery

| | Nozzle | Nozzle | | Insert | | Insert | Insert | |
|---|---|---|---|---|---|---|---|---|
| | Inner | Jet | | Crown | Insert | Inner | Outer | Sump |
| | Diameter | Length | Insert Crown | Depth | Length | Diameter | Diameter | volume |
| Example | (mm) | (mm) | Configuration | (mm) | (mm) | (mm) | (mm) | (μL) |
| 4A | 0.2 | 0.7 | Flat | 0 | 12 | 2 | 4.4 | 25 |
| | | | | | | | tapered | |
| 4B | 0.2 | 0.7 | Flat | 0 | 17 | 2 | 4.4 | 40.7 |
| | | | | | | | tapered | |
| 4C | 0.2 | 0.7 | Flat | 0 | 17 | 1 | 4.4 | 10.3 |
| | | | | | | | tapered | |
| 4D | 0.2 | 0.7 | Flat | 0 | 20 | 1 | 4.4 | 12.7 |
| | | | | | | | tapered | |
| 4E | 0.2 | 0.7 | ϕ1.6 + 90° | 0.5 | 17 | 1 | 4.4 | 9.6 |
| | | | cone | | | | tapered | |
| 4F | 0.2 | 0.7 | ϕ1.6 + 90° | 0.5 | 20 | 1 | 4.4 | 11.9 |
| | | | cone | | | | tapered | |
| 4G | 0.2 | 0.7 | ϕ1 + 90° | 1.5 | 15 | 1 | 4.4 | 7.6 |
| | | | cone + ϕ3 | | | | tapered | |
| 4H | 0.2 | 0.7 | ϕ2.78 sphere | 1.5 | 15 | 1 | 4.4 | 7.6 |
| | | | | | | | tapered | |
| 4I | 0.2 | 0.7 | ϕ3.18 sphere | 1.5 | 15 | 1 | 4.4 | 7.6 |
| | | | | | | | tapered | |
| 4J | 0.2 | 0.7 | ϕ3.18 sphere | 1.5 | 18 | 1 | 4.4 | 9.9 |
| | | | | | | | tapered | |

The MDI actuators of Examples 4A-4J were tested with HCQ inhalation pharmaceutical formulations having HCQ as the API, about 5% alcohol (EtOH), and about 95% propellant HFA-134a. This HCQ inhalation pharmaceutical formulation was a true solution, and each spray dispensed about 200 μg, or 0.2 mg, of HCQ. The MDI actuators of Examples 4A-4J were each connected to a ventilator connector having 55-cm tubing, has an elbow configuration, and did not have an inner channel in proximity to its Luer-lock fitting.

For this study, Examples 4A-4J were compared to a control, which was an MDI configured for stand-alone use, using the same HCQ inhalation pharmaceutical formulation as that used with Examples 4A-4J. The delivery efficiency results are shown in Tables 3-5, below. The Delivery Efficiency Rate was determined by dividing the Total amount (μg) of HCQ Particle Diameter Less Than 1.1 μm per actuation by the HCQ Strength (μg) per actuation.

TABLE 3
HCQ Formulation (200 mcg HCQ base from actuator)

| | HCQ-base | EtOH | HFA | Total | HCQ-base | EtOH % | HFA % |
|---|---|---|---|---|---|---|---|
| Formulation | (g) | (g) | (g) | Weight-g | % w/w | w/w | w/w |
| Each | 0.0503 | 0.5850 | 11.065 | 11.7 | 0.4299% | 5.0000% | 94.5701% |
| Canister | | | | | | | |

TABLE 4
HCQB Delivery Efficiency for Alveoli (Elbow Connection with Inner Channel)

| | Formulation and Device | | | | | Delivered | Delivery |
|---|---|---|---|---|---|---|---|
| Example | API | Actuator | EtOH | Strength | Strength | <1.1 mcm | Rate |
| Hand-held | HCQ Base | O-0.20 | 5% EtOH | 200 mcg | 200 | 89 | 44.5% |
| 4A | HCQ Base | in-Line Tube (55 cm), | 5% EtOH | 200 mcg | 200 | 38.5 | 19.3% |
| | | HCQB-a, connection w/ | | | | | |
| | | inner channel | | | | | |
| 4B | HCQ Base | in-Line Tube (55 cm), | 5% EtOH | 200 mcg | 200 | 53.8 | 26.9% |
| | | HCQB-b, connection w/ | | | | | |
| | | inner channel | | | | | |
| 4C | HCQ Base | in-Line Tube (55 cm), | 5% EtOH | 200 mcg | 200 | 75.8 | 37.9% |
| | | HCQB-c, connection w/ | | | | | |
| | | inner channel, Ave | | | | | |
| 4D | HCQ Base | in-Line Tobe (55 cm), | 5% EtOH | 200 mcg | 200 | 52.8 | 26.4% |
| | | HCQB-d, connection w/ | | | | | |
| | | inner channel | | | | | |
| 4E | HCQ Base | in-Line Tube (55 cm), | 5% EtOH | 200 mcg | 200 | 66.0 | 33.0% |
| | | HCQB-e, connection w/ | | | | | |
| | | inner channel, Ave | | | | | |
| 4F | HCQ Base | in-Line Tube (55 cm), | 5% EtOH | 200 mcg | 200 | 69.4 | 34.7% |
| | | HCQB-f, connection w/ | | | | | |
| | | inner channel, Ave | | | | | |
| 4G | HCQ Base | in-Line Tube (15 cm), | 5% EtOH | 200 mcg | 200 | 68.0 | 34.0% |
| | | HCQB-g, connection w/ | | | | | |
| | | inner channel | | | | | |
| 4H | HCQ Base | in-Line Tube (15 cm), | 5% EtOH | 200 mcg | 200 | 71.7 | 35.9% |
| | | HCQB-h, connection w/ | | | | | |
| | | inner channel | | | | | |
| 4I | HCQ Base | in-Line Tube (15 cm), | 5% EtOH | 200 mcg | 200 | 55.0 | 27.5% |
| | | HCQB-i, connection w/ | | | | | |
| | | inner channel | | | | | |
| 4J | HCQ Base | in-Line Tube (55 cm), | 5% EtOH | 200 mcg | 200 | 60.5 | 30.3% |
| | | HCQB-j, connection w/ | | | | | |
| | | inner channel | | | | | |

TABLE 5
HCQB Delivery Efficiency on Plate 3~5 (Elbow Connection w/ Inner Channel)

| | Formulation and Device | | | | | Delivered | Delivery |
|---|---|---|---|---|---|---|---|
| Example | API | Actuator | EtOH | Strength | Strength | <4.7 mcm | Rate |
| Hand-held | HCQ Base | O-0.20 | 5% EtOH | 200 mcg | 200 | 101.3 | 50.7% |
| 4A | HCQ Base | in-Line Tube (55 cm), | 5% EtOH | 200 mcg | 200 | 26.5 | 13.3% |
| | | HCQB-a, connection w/ | | | | | |
| | | inner channel | | | | | |
| 4B | HCQ Base | in-Line Tube (55 cm), | 5% EtOH | 200 mcg | 200 | 42.6 | 21.3% |
| | | HCQB-b, connection w/ | | | | | |
| | | inner channel | | | | | |
| 4C | HCQ Base | in-Line Tube (55 cm), | 5% EtOH | 200 mcg | 200 | 57.9 | 29.0% |
| | | HCQB-c, connection w/ | | | | | |
| | | inner channel | | | | | |
| 4D | HCQ Base | in-Line Tube (55 cm), | 5% EtOH | 200 mcg | 200 | 46.6 | 23.3% |
| | | HCQB-d, connection w/ | | | | | |
| | | inner channel | | | | | |
| 4E | HCQ Base | in-Line Tube (55 cm), | 5% EtOH | 200 mcg | 200 | 57.9 | 29.0% |
| | | HCQB-e, connection w/ | | | | | |
| | | inner channel | | | | | |
| 4F | HCQ Base | in-Line Tube (55 cm), | 5% EtOH | 200 mcg | 200 | 56.5 | 28.3% |
| | | HCQB-f, connection w/ | | | | | |
| | | inner channel | | | | | |
| 4G | HCQ Base | in-Line Tube (15 cm), | 5% EtOH | 200 mcg | 200 | 60.9 | 30.5% |
| | | HCQB-g, connection w/ | | | | | |
| | | inner channel | | | | | |
| 4H | HCQ Base | in-Line Tube (15 cm), | 5% EtOH | 200 mcg | 200 | 51.6 | 25.8% |
| | | HCQB-h, connection w/ | | | | | |
| | | inner channel | | | | | |
| 4I | HCQ Base | in-Line Tube (15 cm), | 5%, EtOH | 200 mcg | 200 | 45.5 | 22.8% |
| | | HCQB-i, connection w/ | | | | | |
| | | inner channel | | | | | |
| 4J | HCQ Base | in-Line Tube (55 cm), | 5% EtOH | 200 mcg | 200 | 39.9 | 20.0% |
| | | HCQB-j, connection w/ | | | | | |
| | | inner channel | | | | | |

TABLE 6
HCQB Delivery Efficiency for Alveoli (Elbow Connection w/o Inner Channel)

| | Formulation and Device | | | | | Delivered | Delivery |
|---|---|---|---|---|---|---|---|
| Example | API | Actuator | EtOH | Strength | Strength | <1.1 mcm | Rate |
| Hand-held | HCQ Base | O-0.20 | 5% EtOH | 200 mcg | 200 | 89 | 44.5% |
| 4A | HCQ Base | in-Line Tube (15 cm), | 5% EtOH | 200 mcg | 200 | 64.4 | 32.2% |
| | | HCQB-a, connection w/o | | | | | |
| | | inner channel | | | | | |
| 4B | HCQ Base | in-Line Tube (15 cm), | 5% EtOH | 200 mcg | 200 | 62.6 | 31.3% |
| | | HCQB-b, connection w/o | | | | | |
| | | inner channel | | | | | |
| 4C | HCQ Base | in-Line Tube (15 cm), | 5% EtOH | 200 mcg | 200 | 86.6 | 43.3% |
| | | HCQB-c, connection w/o | | | | | |
| | | inner channel | | | | | |
| 4D | HCQ Base | in-Line Tube (15 cm), | 5% EtOH | 200 mcg | 200 | 73.8 | 36.9% |
| | | HCQB-d, connection w/o | | | | | |
| | | inner channel, Ave | | | | | |
| 4E | HCQ Base | in-Line Tube (15 cm), | 5% EtOH | 200 mcg | 200 | 68.4 | 34.2% |
| | | HCQB-e, connection w/o | | | | | |
| | | inner channel | | | | | |
| 4F | HCQ Base | in-Line Tube (15 cm), | 5% EtOH | 200 mcg | 200 | 46.3 | 23.2% |
| | | HCQB-f, connection w/o | | | | | |
| | | inner channel | | | | | |
| 4G | HCQ Base | in-Line Tube (15 cm), | 5% EtOH | 200 mcg | 200 | 63.0 | 31.5% |
| | | HCQB-g, connection w/o | | | | | |
| | | inner channel | | | | | |
| 4H | HCQ Base | in-Line Tube (15 cm), | 5% EtOH | 200 mcg | 200 | 62.1 | 31.1% |
| | | HCQB-h, connection w/o | | | | | |
| | | inner channel | | | | | |
| 4I | HCQ Base | in-Line Tube (15 cm), | 5% EtOH | 200 mcg | 200 | 75.7 | 37.9% |
| | | HCQB-i, connection w/o | | | | | |
| | | inner channel | | | | | |
| 4J | HCQ Base | in-Line Tube (15 cm), | 5% EtOH | 200 mcg | 200 | 58.1 | 29.1% |
| | | HCQB-j, connection w/o | | | | | |
| | | inner channel | | | | | |

As shown in Table 6, Example 4C provided the strongest results among all in-Line actuator with a total amount of 86.6 μg of HCQ particle diameter that are less than 1.1 μm per actuation, and a corresponding delivery efficiency rate of about 43.3%. These results are comparable to the control having a total amount of 89 μg of HCQ particle diameter that are less than 1.1 μm per actuation, and a corresponding delivery efficiency rate of about 44.5%, as shown in Table 6. A particle diameter of less than 1.1 μm is an important because, as discussed above, an alveolus cell has a size of about 0.43 μm to 1.1 μm. More particularly, as shown in the Cascade Impactor illustration of FIG. 2, Stage 6 alveoli have a size of about 0.65 μm to 1.1 μm, and Stage 7 alveoli have a size of about 0.43 μm to 0.65 μm.

Notably, based on these results, the length of the nozzle, and the sump volume are key factors in for a highly efficient delivery of extra-fine API particles. For example, if the nozzle length is too short, such as with Example 4A, then it will cause more API (e.g. HCQ) to be deposited on the elbow connection. By contrast, if the tip is too long, such as with Example 4D, it will cause more API to be deposited in the tubing of the ventilator connector. With respect to the sump volume, a smaller volume, for example with Examples 4B-4C, increases the delivery efficiency of the API.

As demonstrated by the aforementioned Examples and experimental data, the disclosed aerosol drug delivery devices advantageously provide particle size control and a highly efficient target site delivery of inhalation pharmaceutical formulations. In particular, the disclosed devices are configured to enable the production of fine API particle sizes having a particle diameter of less than 4.7 μm, and the extra-fine API particles having a particle diameter of less than 1.1 μm.

Further, by producing fine and extra-fine API particles, the disclosed devices can provide a highly efficient target site delivery. Specifically, the disclosed devices can deliver fine and extra-fine API particles to a respiratory track and into deep, peripheral lungs, alveoli, or alveoli lining fluid, thereby enabling the fine and extra-fine API particles to take effect right on one or more lesions in the respiratory track and into deep, peripheral lungs, alveoli, or alveoli lining fluid. This feature is advantageous because it allows the disclosed devices and methods to effectively treat a pulmonary disease that can affect a mechanically ventilated patient's lungs, especially a pulmonary disease that affects the deep, peripheral lungs, alveoli, or alveoli lining fluid, such as COVID-19.

Thus, in some embodiments, the disclosed devices provide a delivery efficiency of no less than 60% of fine API particles to the patient's respiratory track, and the respective delivery efficiency is determined by dividing (i) a total amount of the API having the respective particle diameter by (ii) an expected metered dose of the API. In other embodiments, the delivery efficiency rate is at least 50%, 55%, 65%, 70%, 75%, or more to the patient's respiratory track.

Further, in some embodiments, the disclosed devices provide a delivery efficiency of no less than 30% of the extra-fine API particles to the patient's deep, peripheral lungs, alveoli, or alveoli lining fluid, and the respective delivery efficiency is determined by dividing (i) a total amount of the API having the respective particle diameter by (ii) an expected metered dose of the API. In some embodiments, the delivery efficiency rate is at least 20%, 25%, 35%, 40%, 45%, 50%, or more to the patient's deep, peripheral lungs, alveoli, or alveoli lining fluid.

### Pharmaceutical Formulations for Providing High Targeted Delivery Efficiency Rates

As described above, it has been determined that inhaled API's are effective in the treatment of COVID-19. As used herein, the terms “treating” or “treatment” refer to reducing severity, eliminating, or a combination thereof, with respect to a particular disease, condition, or injury. Thus, in the context of the disclosed methods of treatment of COVID-19, the disclosed methods are intended to: (i) reduce severity, (ii) eliminate, or (iii) reduce severity and eliminate COVID-19. As described, common symptoms of COVID-19 include dry cough, difficulty breathing (e.g. shortness of breath), fever (e.g. body temperature of 100.4° Fahrenheit or more), fatigue, and others. Thus, the disclosed methods for treating COVID-19 may reduce and/or eliminate some of these symptoms of COVID-19 over a specified period of time.

In some embodiments, the API used in conjunction with the disclosed MDI's includes an anti-viral therapeutic agent for treating a pulmonary disease, for example HCQ, a free base thereof, or a pharmaceutically acceptable salt thereof. For brevity throughout this disclosure, “HCQ pharmaceutical formulation” or “HCQ formulation” refers to a pharmaceutical formulation having HCQ, a free base thereof, or a pharmaceutically acceptable salt thereof, as the API.

In some embodiments, the API used in conjunction with the disclosed MDI's includes an inhalable steroid or bronchodilator for treating a pulmonary disease. Non-limiting examples of inhalable steroids include flunisolide, fluticasone furoate, fluticasone propionate, triamcinolone acetonide, beclomethasone dipropionate, budesonide, mometasone furoate, ciclesonide, or pharmaceutically acceptable salts thereof. Non-limiting examples of bronchodilators include albuterol, levosalbutamol, pirbuterol, epinephrine, racemic epinephrine, ephedrine, terbutaline, salmeterol, formoterol, bambuterol, indacaterol or pharmaceutically acceptable salts thereof. In some embodiments, the API is therapeutically effective in treating asthma, chronic obstructive pulmonary disease (COPD), sarcoidosis, eosinophilic pneumonia, pneumonia, interstitial lung disease, bronchiolitis, bronchiectasis, or restrictive lung diseases.

In some embodiments, the API further includes a propellant, where the API is dissolved in the propellant at a pre-determined ratio, with or without a co-solvent, and where the pharmaceutical formulation is for administration by inhalation. In some embodiments, the formulation further includes a co-solvent, such as an alcohol.

In some embodiments, the therapeutically effective dose of the API is the dose per treatment that is therapeutically effective in treating a pulmonary disease, for example COVID-19. As will be described further below, the therapeutically effective dose of the API, such as HCQ, can be dispensed in one or more metered doses of the pharmaceutical formulation from the MDI. A single metered dose is the dose of the API dispensed per actuation (or per spray) from the MDI using an MDI actuator.

Thus, in some embodiments, the pharmaceutical formulation further comprises 0.25% to 1.50% (w/w) HCQ; 3.00% to 15.00% (w/w) of a co-solvent, such as an alcohol; and 80.00% to 97.00% (w/w) of a propellant; wherein “w/w” denotes weight by weight. In further embodiments, the pharmaceutical formulation further comprises 0.25% to 1.50% (w/w) HCQ; 3.00% to 15.00% (w/w) ethanol; 80.00% to 97.00% (w/w) of a propellant, wherein the propellant is HFA 134a (“w/w” denotes weight by weight).

In some embodiments, the pharmaceutical formulation further comprises 0.40% to 0.50% (w/w) of an HCQ free base; 4.00% to 8.00% (w/w) ethanol; and 93.00% to 96.00% (w/w) HFA propellant; wherein the formulation is a true solution. In further embodiments, the pharmaceutical formulation further comprises a propellant, wherein the propellant is HFA; and the HCQ is dissolved in the HFA at a pre-determined ratio, with or without co-solvent. In some embodiments, the formulation further comprises a surfactant. Non-limiting examples of surfactants include polyethylene glycol (PEG), PEG 300, PEG 600, PEG 1000, Brij 30, Brij 35, Brij 56, Brij 76, Brij 97, polysorbate (Tween), Tween 20, Tween 60, Tween 80, polypropylene glycol (PPG), PPG 2000, Pluronic 10-R5, Pluronic 17-R2, Pluronic 25-R4, Pluronic F-68, Pluronic F-127, Pluronic L-43, Pluronic L-44 NF, Pluronic L-62, Pluronic L-64, Pluronic L-101, polyvinyl pyrrolidone K25, polyvinylalcohol, aerosol OT (sodium dioctyl sulfosuccinate), oleic acid, oligolactic acid, lecithin, Span 20, Span 80, Span 85, and combinations thereof.

In some embodiments, the therapeutically effective dose of the API is in the range of 0.5 mg to 5.0 mg, including subranges, such as 0.5 mg to 4.5 mg, 0.5 mg to 4.0 mg, 0.5 mg to 3.5 mg, 0.5 mg to 3.0 mg, 0.5 mg to 2.5 mg, 0.5 mg to 2.0 mg, 0.5 mg to 1.5 mg, 0.5 mg to 4.5 mg, 0.5 mg to 4.0 mg, 0.5 mg to 3.5 mg, 0.5 mg to 3.0 mg, 0.5 mg to 2.5 mg, 0.5 mg to 2.0 mg, 0.5 mg to 1.5 mg, 1.0 mg to 5.0 mg, 1.0 mg to 4.5 mg, 1.0 mg to 4.0 mg, 1.0 mg to 3.5 mg, 1.0 mg to 3.0 mg, 1.0 mg to 2.5 mg, 1.0 mg to 2.0 mg, 1.0 mg to 1.5 mg, 1.5 mg to 5.0 mg, 1.5 mg to 4.5 mg, 1.5 mg to 4.0 mg, 1.5 mg to 3.5 mg, 1.5 mg to 3.0 mg, 1.5 mg to 2.5 mg, 1.5 mg to 2.0 mg, 2.0 mg to 5.0 mg, 2.0 mg to 4.5 mg, 2.0 mg to 4.0 mg, 2.0 mg to 3.5 mg, 2.0 mg to 3.0 mg, 2.0 mg to 2.5 mg, 2.5 mg to 5.0 mg, 2.5 mg to 4.5 mg, 2.5 mg to 4.0 mg, 2.5 mg to 3.5 mg, 2.0 mg to 3.0 mg, 3.0 mg to 5.0 mg, 3.0 mg to 4.5 mg, 3.0 mg to 4.0 mg, 3.0 mg to 3.5 mg, 3.5 mg to 5.0 mg, 3.5 mg to 4.5 mg, 3.5 mg to 4.0 mg, 4.0 mg to 5.0 mg, 4.0 mg to 4.5 mg, or 4.5 mg to 5.0 mg, In some embodiments, the therapeutically effective dose of the API, such as HCQ, is 0.5 mg to 2.5 mg, 1.0 mg to 2.0 mg, including about 1.0 mg or about 2.0 mg.

In some embodiments, where the patient has at least a mild COVID-19 infection, such as a mild to moderate COVID-19 infection, the therapeutically effective dose is 0.5 mg to 3.0 mg of the anti-viral therapeutic agent, for example HCQ. In some embodiments, a patient having at least a mild COVID-19 infection does not require airway support for breathing. In some embodiments, for a patient having at least a mild COVID-19 infection, the therapeutically effective dose is about 1.0 mg of the anti-viral therapeutic agent, for example HCQ.

In some embodiments, where the patient has a severe COVID-19 infection, the therapeutically effective dose is in the range of 1.5 mg to 5.0 mg of the anti-viral therapeutic agent, such as HCQ. In some embodiments, the therapeutically effective dose is in the range of 1.5 mg to 4.0 mg. In some embodiments, the patient having COVID-19 is operatively connected to a ventilator. In other embodiments, the patient having COVID-19 does not require airway support for breathing. In some embodiments, the patient has severe COVID-19, and the therapeutically effective dose is about 2.0 mg of the anti-viral therapeutic agent, such as HCQ.

In some embodiments, the therapeutically effective dose of the API, such as HCQ, is administered in one or more metered dose. A single metered is the dose of the API dispensed per actuation (or per spray) from the MDI using an MDI actuator. Thus, in some embodiments, a single metered dose of the API, such as HCQ, is 0.05 mg to 1.00 mg, or any range, including subranges, such as 0.10 mg to 0.90 mg, 0.10 mg to 0.80 mg, 0.10 mg to 0.70 mg, 0.10 mg to 0.60 mg, 0.10 mg to 0.50 mg, 0.10 mg to 0.40 mg, 0.10 mg to 0.30 mg, 0.10 mg to 0.20 mg, 0.20 mg to 1.00 mg, 0.20 mg to 0.90 mg, 0.20 mg to 0.80 mg, 0.20 mg to 0.70 mg, 0.20 mg to 0.60 mg, 0.20 mg to 0.50 mg, 0.20 mg to 0.40 mg, 0.20 mg to 0.30 mg, 0.30 mg to 1.00 mg, 0.30 mg to 0.90 mg, 0.30 mg to 0.80 mg, 0.30 mg to 0.70 mg, 0.30 mg to 0.60 mg, 0.30 mg to 0.50 mg, 0.30 mg to 0.40 mg, 0.40 mg to 1.00 mg, 0.40 mg to 0.90 mg, 0.40 mg to 0.80 mg, 0.40 mg to 0.70 mg, 0.40 mg to 0.60 mg, 0.40 mg to 0.50 mg, 0.50 mg to 1.00 mg, 0.50 mg to 0.90 mg, 0.50 mg to 0.80 mg, 0.50 mg to 0.70 mg, 0.50 mg to 0.60 mg, 0.60 mg to 1.00 mg, 0.60 mg to 0.90 mg, 0.60 mg to 0.80 mg, 0.60 mg to 0.70 mg, 0.70 mg to 0.90 mg, 0.70 mg to 0.80 mg, 0.80 mg to 0.90 mg, or 0.90 mg to 1.0 mg,

In some embodiments, a single metered dose of the API, such as HCQ, is 0.05 mg to 1.00 mg, or about 0.40 mg.

In still other embodiments, a single metered dose of the API, such as HCQ, is at least about 0.10 mg, at least about 0.20 mg, at least about 0.30 mg, at least about 0.40 mg, at least about 0.50 mg, at least about 0.60 mg, at least about 0.70 mg, at least about 0.80 mg, at least about 0.90 mg, or at least about 1.00 mg. In some embodiments, a single metered dose of the API, such as HCQ, is at least about 0.20 mg.

In some embodiments, the therapeutically effective dose of the API, such as HCQ, can be dispensed in one or more metered doses. Thus, in some embodiments, the therapeutically effective dose of the API, such as HCQ, can be dispensed in 2, 3, 4, 5, 6, 7, 8, 9, 10, 11, 12, 13, 14, 15, or more metered doses to arrive at the desired therapeutically effective dose.

In some embodiments of the HCQ pharmaceutical formulation, the HCQ includes a HCQ free base. In further embodiments, based on the total weight of the formulation, the HCQ, such as HCQ free base, is 0.25% to 1.50% (w/w), including subranges, such as 0.25% to 1.25% (w/w), 0.25% to 1.00% (w/w), 0.25% to 0.75% (w/w), 0.25% to 0.50/0 (w/w), 0.30% to 1.50% (w/w), 0.30% to 1.25% (w/w), 0.30% to 1.00% (w/w), 0.30% to 0.75% (w/w), 0.30% to 0.50% (w/w), 0.35% to 1.50% (w/w), 0.35% to 1.25% (w/w), 0.35% to 1.00% (w/w), 0.35% to 0.75% (w/w), 0.35% to 0.50% (w/w), 0.40% to 1.50% (w/w), 0.40% to 1.25% (w/w), 0.40% to 1.00% (w/w), 0.40% to 0.75% (w/w), 0.40% to 0.50% (w/w), 0.45% to 1.50% (w/w), 0.45% to 1.25% (w/w), 0.45% to 1.00% (w/w), 0.45% to 0.75% (w/w), 0.45% to 0.50% (w/w), 0.50% to 1.50% (w/w), 0.50% to 1.25% (w/w), 0.50% to 1.00% (w/w), 0.50% to 0.75% (w/w), 0.60% to 1.50% (w/w), 0.60% to 1.25% (w/w), 0.60% to 1.00% (w/w), 0.60% to 0.75% (w/w), 0.65% to 1.50% (w/w), 0.65% to 1.25% (w/w), 0.65% to 1.00% (w/w), 0.65% to 0.75% (w/w), 0.70% to 1.50% (w/w), 0.70% to 1.25% (w/w), 0.70% to 1.00% (w/w), 0.75% to 1.50% (w/w), 0.75% to 1.25% (w/w), 0.75% to 1.00% (w/w), 0.80% to 1.50% (w/w), 0.80% to 1.25% (w/w), 0.80% to 1.00% (w/w), 0.85% to 1.50% (w/w), 0.85% to 1.25% (w/w), 0.85% to 1.00% (w/w), 0.90% to 1.50% (w/w), 0.90% to 1.25% (w/w), or 0.90% to 1.00% (w/w). In further embodiments, based on the total weight of the formulation, the HCQ, such as HCQ free base, is about 0.38% (w/w), about 0.44% (w/w), about 0.54% (w/w), about 0.60% (w/w), about 0.76% (w/w), or about 1.08% (w/w).

In some embodiments, the HCQ includes HCQ free base, and, the HCQ, such as HCQ free base, is 0.30% to 1.25% (w/w) based on the total weight of the formulation, including but not limited to about 0.38% (w/w), about 0.44% (w/w), about 0.54% (w/w), about 0.60% (w/w), about 0.76% (w/w), or about 1.08% (w/w).

In some embodiments of the pharmaceutical formulation, the formulation further includes a co-solvent, such as an alcohol, the alcohol includes ethanol. In further embodiments, based on the total weight of the formulation, the co-solvent, such as ethanol, is 3.00% to 15.00% (w/w), including subranges, such as 3.00% to 12.50% (w/w), 3.00% to 10.0% (w/w), 3.00% to 8.50% (w/w), 3.00% to 7.50% (w/w), 3.00% to 6.50% (w/w), 3.00% to 6.25% (w/w), 3.00% to 5.75% (w/w), 3.00% to 5.25% (w/w), 3.00% to 4.75% (w/w), 3.00% to 4.50% (w/w), 3.00% to 4.25% (w/w), 3.00% to 4.00% (w/w), 3.50% to 12.50% (w/w), 3.50% to 10.0% (w/w), 3.50% to 8.50% (w/w), 3.50% to 7.50% (w/w), 3.50% to 6.50% (w/w), 3.50% to 6.25% (w/w), 3.50% to 5.75% (w/w), 3.50% to 5.25% (w/w), 3.50% to 4.75% (w/w), 3.50% to 4.50% (w/w), 3.50% to 4.25% (w/w), 3.50% to 4.00% (w/w), 4.00% to 12.50% (w/w), 4.00% to 10.0% (w/w), 4.00% to 8.50% (w/w), 4.00% to 7.50% (w/w), 4.00% to 6.50% (w/w), 4.00% to 6.25% (w/w), 4.00% to 5.75% (w/w), 4.00% to 5.25% (w/w), 4.00% to 4.75% (w/w), 4.00% to 4.50% (w/w), 4.00% to 4.25% (w/w), 4.50% to 12.50% (w/w), 4.50% to 10.0% (w/w), 4.50% to 8.50% (w/w), 4.50% to 7.50% (w/w), 4.50% to 6.50% (w/w), 4.50% to 6.25% (w/w), 4.50% to 5.75% (w/w), 4.50% to 5.25% (w/w), 4.50% to 4.75% (w/w), 5.00% to 12.50% (w/w), 5.00% to 10.0% (w/w), 5.00% to 8.50% (w/w), 5.00% to 7.50% (w/w), 5.00/6 to 6.50% (w/w), 5.00% to 6.25% (w/w), 5.00% to 5.75% (w/w), 5.00% to 5.25% (w/w), 5.50% to 12.50% (w/w), 5.50% to 10.0% (w/w), 5.50% to 8.50% (w/w), 5.50% to 7.50% (w/w), 5.50% to 6.50% (w/w), 5.50% to 6.25% (w/w), 5.50% to 5.75% (w/w), 6.00% to 12.50% (w/w), 6.00% to 10.0% (w/w), 6.00% to 8.50% (w/w), 6.00% to 7.50% (w/w), 6.00% to 6.50% (w/w), 6.00% to 6.25% (w/w), 7.50% to 12.50% (w/w), 7.50% to 10.0% (w/w), 7.50% to 8.50% (w/w), 10.0% to 15.00% (w/w), or 10.00% to 13.0% (w/w). In further embodiments, based on the total weight of the formulation, the co-solvent, such as alcohol and ethanol, is about 4.00% (w/w), about 4.50% (w/w), about 5.00% (w/w), about 5.50% (w/w), about 6.00% (w/w), about 8.00% (w/w), or about 12.00% (w/w).

In some embodiments, the co-solvent includes alcohol, such as ethanol, and the ethanol is 3.50% to 12.50% (w/w) based on the total weight of the formulation, including but not limited to about 4.00 (w/w), about 4.50% (w/w), about 5.00% (w/w), about 5.50% (w/w), about 6.00% (w/w), about 8.00% (w/w), or about 12.00% (w/w).

In some embodiments of the pharmaceutical formulation, the propellant includes HFA 134a. In further embodiments, based on the total weight of the formulation, the propellant, such as HFA 134a, is 80.00% to 97.00% (w/w), including subranges, such as 80.00% to 95.00% (w/w), 80.00% to 94.50% (w/w), 80.00% to 94.00% (w/w), 80.00% to 93.50% (w/w), 80.00% to 93.00% (w/w), 80.00% to 92.50% (w/w), 80.00% to 92.00% (w/w), 80.00% to 91.50% (w/w), 80.00% to 90.0% (w/w), 85.00% to 95.00% (w/w), 85.00% to 94.50% (w/w), 85.00% to 94.00% (w/w), 85.00% to 93.50% (w/w), 85.00% to 93.00% (w/w), 85.00% to 92.50% (w/w), 85.00% to 92.00% (w/w), 85.00% to 91.50% (w/w), 85.00% to 90.00% (w/w), 90.00% to 95.00% (w/w), 90.00% to 94.50% (w/w), 90.00% to 94.00% (w/w), 90.00% to 93.50% (w/w), 90.00% to 93.00% (w/w), 90.00% to 92.50% (w/w), 90.00% to 92.00% (w/w), 90.00% to 91.50% (w/w), 93.50% to 95.00% (w/w), 93.50% to 94.50% (w/w), or 93.50% to 94.00% (w/w). In further embodiments, based on the total weight of the formulation, the propellant, such as HFA 134a, is about 86.92% (w/w), about 91.24% (w/w), about 93.40% (w/w), about 94.06% (w/w), about 94.46% (w/w), about 94.56% (w/w), about 94.57% (w/w), about 94.62% (w/w), about 95.06% (w/w), or about 95.62% (w/w).

In some embodiments, the propellant is HFA 134a, and the HFA 134a is 85.00% to 95.00% (w/w) based on the total weight of the formulation, including but not limited to about 86.92% (w/w), about 91.24% (w/w), about 93.36% (w/w), about 94.06% (w/w), about 94.46% (w/w), about 94.56% (w/w), about 94.62% (w/w), about 95.06% (w/w), or about 95.62% (w/w).

In some embodiments, the propellant is HFA 152a, isobutane, HFO, HFO 1234ze (Solstice™), HFO 1234yf (Opteon™), HFA 227, a mixture of HFA 134a and HFA 227, or a combination thereof.

In some embodiments, the HCQ is dissolved in the propellant at a pre-determined ratio. The various pre-determined ratios can be ascertained based on the aforementioned described weights of the HCQ and the propellant. In some embodiments, based on the total weight of the formulation, the HCQ, such as HCQ free base, is 0.43% (w/w) and the propellant, such as HFA 134a, is 94.57% (w/w), and thus, the pre-determined ratio of the propellant to HFA about 219.93 to 1.

In some embodiments, the total weight of the pharmaceutical formulation is about 10.0-15.0 grams. In some embodiments, the total weight of the pharmaceutical formulation is about 11.7 grams.

In some embodiments of the pharmaceutical formulation, the formulation includes a true solution. In one embodiment, the formulation includes a true solution.

In some embodiments, the pharmaceutical formulation is in a MDI, and each metered-dose, per actuation, of the API is 150 μg to 600 μg, including subranges, such as 150 μg to 550 μg, 150 μg to 525 μg, 150 μg to 450 μg, 150 μg to 400 μg, 150 μg to 375 μg, 150 μg to 350 μg, 150 μg to 325 μg, 150 μg to 280 μg, 150 μg to 260 μg, 150 μg to 240 μg, 150 μg to 220 μg, 150 μg to 210 μg, 150 μg to 190 μg, 170 μg to 550 μg, 170 μg to 525 μg, 170 μg to 450 μg, 170 μg to 400 μg, 170 μg to 375 μg, 170 μg to 350 μg, 150 μg to 325 μg, 170 μg to 280 μg, 170 μg to 260 μg, 170 μg to 240 μg, 170 μg to 220 μg, 170 μg to 210 μg, 170 μg to 190 μg, 190 μg to 550 μg, 190 μg to 525 μg, 190 μg to 450 μg, 190 μg to 400 μg, 190 μg to 375 μg, 190 μg to 350 μg, 190 μg to 325 μg, 190 μg to 280 μg, 190 μg to 260 μg, 190 μg to 240 μg, 190 μg to 220 μg, 190 μg to 210 μg, 200 μg to 550 μg, 200 μg to 525 μg, 200 μg to 450 μg, 200 μg to 400 μg, 200 μg to 375 μg, 200 μg to 350 μg, 200 μg to 325 μg, 200 μg to 280 μg, 200 μg to 260 μg, 200 μg to 240 μg, 200 μg to 220 μg, 200 μg to 210 μg, 225 μg to 550 μg, 225 μg to 525 μg, 225 μg to 450 μg, 225 μg to 400 μg, 225 μg to 375 μg, 225 μg to 350 μg, 225 μg to 325 μg, 225 μg to 280 μg, 225 μg to 260 μg, 225 μg to 240 μg, 240 μg to 550 μg, 240 μg to 525 μg, 240 μg to 450 μg, 240 μg to 400 μg, 240 μg to 375 μg, 240 μg to 350 μg, 240 μg to 325 μg, 240 μg to 280 μg, 240 μg to 260 μg, 250 μg to 550 μg, 250 μg to 525 μg, 250 μg to 450 μg, 250 μg to 400 μg, 250 μg to 375 μg, 250 μg to 350 μg, 250 μg to 325 μg, 250 μg to 280 μg, 250 μg to 260 μg, 270 μg to 550 μg, 270 μg to 525 μg, 270 μg to 450 μg, 270 μg to 400 μg, 270 μg to 375 μg, 270 μg to 350 μg, 270 μg to 325 μg, 270 μg to 280 μg, 300 μg to 550 μg, 300 μg to 525 μg, 300 μg to 450 μg, 300 μg to 400 μg, 300 μg to 375 μg, 270 μg to 350 μg, 300 μg to 325 μg, 350 μg to 550 μg, 350 μg to 525 μg, 350 μg to 450 μg, 350 μg to 400 μg, 350 μg to 375 μg, 400 μg to 550 μg, 400 μg to 525 μg, or 400 μg to 450 μg. In further embodiments, the pharmaceutical formulation is in a MDI, and each metered-dose, per actuation, of the API is about 150 μg, about 175 μg, about 200 μg, about 205 μg, about 225 μg, about 250 μg, about 275 μg, about 300 μg, about 325 μg, about 350 μg, about 375 μg, about 400 μg, about 425 μg, about 450 μg, about 475 μg, or about 500 μg.

In some embodiments, the pharmaceutical formulation is in a MDI, and each metered-dose, per actuation, of the API is 170 μg to 525 μg, including but not limited to, about 175 μg, about 200 μg, about 205 μg, about 250 μg, about 275 μg, about 350 μg, about 400 μg, about 450 μg, or about 500 μg.

In some embodiments, the pharmaceutical formulation is in a MDI, and each metered-dose, per actuation, of the API is 600 μg to 850 μg, including subranges, such as 600 μg to 625 μg, 600 μg to 650 μg, 600 μg to 675 μg, 600 μg to 700 μg, 600 μg to 725 μg, 600 μg to 750 μg, 600 μg to 775 μg, 600 μg to 800 μg, 600 μg to 825 μg, 600 μg to 850 μg, 625 μg to 650 μg, 625 μg to 675 μg, 625 μg to 700 μg, 625 μg to 725 μg, 625 μg to 750 μg, 625 μg to 775 μg, 625 μg to 800 μg, 625 μg to 825 μg, 625 μg to 850 μg, 650 μg to 675 μg, 650 μg to 700 μg, 650 μg to 725 μg, 650 μg to 750 μg, 650 μg to 775 μg, 650 μg to 800 μg, 650 μg to 825 μg, 650 μg to 850 μg, 675 μg to 700 μg, 675 μg to 725 μg, 675 μg to 750 μg, 675 μg to 775 μg, 675 μg to 800 μg, 675 μg to 825 μg, 675 μg to 850 μg, 700 μg to 725 μg, 700 μg to 750 μg, 700 μg to 775 μg, 700 μg to 800 μg, 700 μg to 825 μg, 700 μg to 850 μg 725 μg to 750 μg, 725 μg to 775 μg, 725 μg to 800 μg, 725 μg to 825 μg, 725 μg to 850 μg, 750 μg to 775 μg, 750 μg to 800 μg, 750 μg to 825 μg, 750 μg to 850 μg, 775 μg to 800 μg, 775 μg to 825 μg, 775 μg to 850 μg, 800 μg to 825 μg, 800 μg to 850 μg, or 825 μg to 850 μg. In further embodiments, the pharmaceutical formulation is in a MDI, and each metered-dose, per actuation, of the API is about 625 μg, about 650 μg, about 675 μg, about 700 μg, about 725 μg, about 750 μg, about 775 μg, about 800 μg, about 825 μg, or about 850 μg.

In some embodiments, the dose, such as the therapeutically effective dose, of HCQ, is 0.5 mg to 5.0 mg, including subranges, such as 0.5 mg to 4.5 mg, 0.5 mg to 4.0 mg, 0.5 mg to 3.5 mg, 0.5 mg to 3.0 mg, 0.5 mg to 2.5 mg, 0.5 mg to 2.0 mg, 0.5 mg to 1.5 mg, 0.5 mg to 4.5 mg, 0.5 mg to 4.0 mg, 0.5 mg to 3.5 mg, 0.5 mg to 3.0 mg, 0.5 mg to 2.5 mg, 0.5 mg to 2.0 mg, 0.5 mg to 1.5 mg, 1.0 mg to 5.0 mg, 1.0 mg to 4.5 mg, 1.0 mg to 4.0 mg, 1.0 mg to 3.5 mg, 1.0 mg to 3.0 mg, 1.0 mg to 2.5 mg, 1.0 mg to 2.0 mg, 1.0 mg to 1.5 mg, 1.5 mg to 5.0 mg, 1.5 mg to 4.5 mg, 1.5 mg to 4.0 mg, 1.5 mg to 3.5 mg, 1.5 mg to 3.0 mg, 0.5 mg to 2.5 mg, 1.5 mg to 2.0 mg, 2.0 mg to 5.0 mg, 2.0 mg to 4.5 mg, 2.0 mg to 4.0 mg, 2.0 mg to 3.5 mg, 2.0 mg to 3.0 mg, 2.0 mg to 2.5 mg, 2.5 mg to 5.0 mg, 2.5 mg to 4.5 mg, 2.5 mg to 4.0 mg, 2.5 mg to 3.5 mg, 2.0 mg to 3.0 mg, 3.0 mg to 5.0 mg, 3.0 mg to 4.5 mg, 3.0 mg to 4.0 mg, 3.0 mg to 3.5 mg, 3.5 mg to 5.0 mg, 3.5 mg to 4.5 mg, 3.5 mg to 4.0 mg, 4.0 mg to 5.0 mg, 4.0 mg to 4.5 mg, or 4.5 mg to 5.0 mg, In some embodiments, the dose, such as the therapeutically effective dose, of HCQ is 0.5 mg to 2.5 mg and 1.0 mg to 2.0 mg.

In other embodiments, the dose, such as the therapeutically effective dose, of HCQ, is about 0.50 mg, about 0.75 mg, about 1.00 mg, about 1.25 mg, about 1.50 mg, about 1.75 mg, about 2.00 mg, about 2.25 mg, about 2.50 mg, about 3.00 mg, about 3.25 mg, about 3.50 mg, about 3.75 mg, about 4.00 mg, about 4.25 mg, about 4.50 mg, about 4.75 mg, or about 5.00 mg. In some embodiments, the dose, such as the therapeutically effective dose, of HCQ is about 1.0 mg or about 2.0 mg.

In some embodiments, the dose or therapeutically effective dose of the API, such as HCQ, can be dispensed in one or more actuations (or sprays). Thus, in some embodiments, the dose or therapeutically effective dose of HCQ can be dispensed in 2, 3, 4, 5, 6, 7, 8, 9, 10, 11, 12, 13, 14, 15, or more actuations in order to arrive at the desired dose.

In some embodiments of the HCQ pharmaceutical formulations, the therapeutically effective dose for treating mild to moderate COVID-19 patients is about 1.0 mg of HCQ, which can be dispensed by one or more actuations. In some embodiments of the HCQ pharmaceutical formulations, the therapeutically effective dose for treating mild to moderate COVID-19 patients is about 1.0 mg of HCQ, which is dispensed in 5 actuations, and each actuation dispenses about 0.2 mg of HCQ.

In some embodiments of the HCQ pharmaceutical formulations, the therapeutically effective dose for treating severe COVID-19 patients is about 2.0 mg of HCQ, which can be dispensed by one or more actuations. In some embodiments of the HCQ pharmaceutical formulations, the therapeutically effective dose for treating a severe COVID-19 patient by administering, via inhalation, a dose of about 2.0 mg of HCQ, which is dispensed in 10 actuations, and each actuation dispenses about 0.2 mg of HCQ.

In some embodiments, the weights of the various ingredients of the formulation and the total weight of the formulation is determined at the time of the release of the formulation for use, sale, or distribution. As background, a drug product, which includes its pharmaceutical formulation, has certain release specifications that a manufactured drug product, including its pharmaceutical formulation, must pass in order to be released for sale, distribution, or use.

In some embodiments, the pharmaceutical formulation provides a long shelf-life due to the formulation being highly stable. Thus, in some embodiments, the disclosed formulation can have a shelf-life including, but not limited to, 3-months, 6-months, 9-months, 12-months, 15-months, 18-months, 21-months, 24-months, or longer after the release of the drug product for sale, distribution, or use.

In some embodiments of the HCQ pharmaceutical formulation, the formulation is efficient in terms of the number of total formulation components. Thus, in some embodiments, the formulation includes only four components, namely HCQ as the API, alcohol, a surfactant, and the propellant.

For brevity, other corresponding embodiments of the disclosed methods have already been described in detail with respect to the description of the disclosed MDI actuators and its various functions.

In some embodiments, the therapeutically effective dose of the anti-viral therapeutic agent is intended for substantially non-systemic delivery to lower systemic exposure of the anti-viral therapeutic agent, and cause less adverse drug events (“ADE”) compared to a same or a different anti-viral therapeutic agent using a different route of administration, as will be described further below.

In some embodiments, wherein the therapeutically effective dose of the anti-viral therapeutic agent is intended for substantially non-systemic delivery to lower systemic exposure of the anti-viral therapeutic agent, and lower risk of overdose toxicity compared to a same or a different anti-viral therapeutic agent using a different route of administration, as will be described further below.

In some embodiments, the lower systemic exposure of the anti-viral therapeutic agent is compared to an oral administration of a tablet comprising an API, wherein the API is HCQ or chloroquine (“CQ”).

In some embodiments, the anti-viral therapeutic agent is hydroxychloroquine (“HCQ”), a free base thereof, or a pharmaceutically acceptable salt thereof, as will be described further below. In some embodiments, the HCQ has a favorable half maximal effective concentration (“EC50”) compared to other anti-viral therapeutic agents including HCQ oral tablet, CQ oral tablet, ribavirin, and remdesivir.

Comparison of EC50 of HCQ with that of Other Anti-Viral Agents

As will be shown in Table 7, below, an “EC50” of a drug represents the anti-viral capability of that drug. More specifically, EC50 is the half maximal effective concentration, which refers to the concentration of a drug, antibody or toxicant which induces a response halfway between the baseline and maximum after a specified exposure time. Therefore, EC50 represents the concentration of a compound where 50% of its maximal effect is observed. Table 7 lists EC50's of a group of anti-viral agents that have been recently discussed in the literature and clinical studies to combat COVID-19.

TABLE 7
EC50 Towards SARS-COV-2 with HCQ, CQ and
Other Drug Products for Oral Tablet Treatment

| | | EC 50, for SARS-Cov2, μM | | | | |
|---|---|---|---|---|---|---|
| | Drug | | | Prophylactic | | |
| | Product | Treatment | | Treatment | | |
| No. | Name | 24 hrs | 48 hrs | 24 hrs | 48 hrs | Reference |
| 1 | Ribavirin | — | 105.9 | — | — | [4] |
| 2 | Remdesivir | — | 0.77 | — | — | |
| 3 | CQ | — | 1.13 | — | — | |
| 4 | CQ | 23.9 | 5.47 | >100 | 18.0 | [3] |
| 5 | HCQ | 6.14 | 0.72 | 6.25 | 5.85 | |
| Ratio of | | 3.9 | 7.6 | >16 | 3.1 | |
| CQ:HCQ | | | | | | |

As demonstrated in Table 7, HCQ possesses a favorable EC50 compared to other anti-viral agents. Inhibition of DNA and RNA polymerase reaction by CQ has been described as the ability of chloroquine to bind to both DNA and RNA in vitro, suggesting a possible mechanism by which this drug interferes with cellular processes in malarial parasites. Accordingly, as shown in Table 7, the less toxic HCQ attracted more attention than CQ. The primary anti-viral mechanism of HCQ is the premature termination of RNA transcription of CoV2, resulting in a disabling of CoV2 replication process.

HCQ has a stronger inhibition ability for SARS-CoV-2 (EC50=6.14 μM, 24 hrs test) than that for CQ (EC50=23.9 μM, 24 hrs test) and other potential anti-viral drugs, such as Ribavirin and Remdesivir, as summarized in Table 7.

Table 7 includes two sets of EC50 data conducted by two studies. When different methods are used, the EC50 data may not be same. However, within one study, the EC50 for different drugs can be compared to find the relative anti-viral activity.

The EC50 data in Table 7 provides results of multiple potential anti-viral drugs that were tested against CoV2 and demonstrate that HCQ is one of the drugs with the strongest anti-viral activity towards CoV2. For example, CQ has an EC50 that is comparable to Remdesivir, and our study indicated that HCQ has an EC50 that is 3.9 times lower than CQ, namely HCQ's anti-viral ability towards CoV2 is 3.9 or 7.6 times stronger than CQ for in vitro treatment after 24 and 48 hrs, respectively.

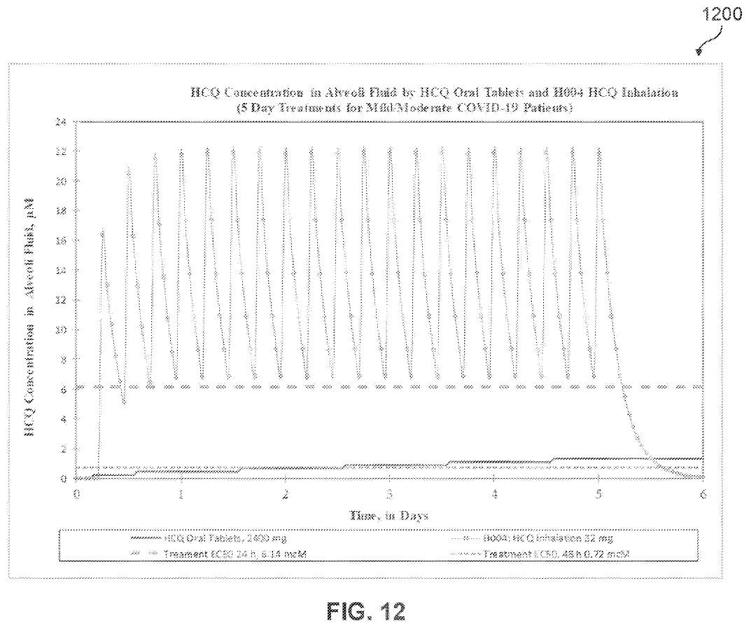

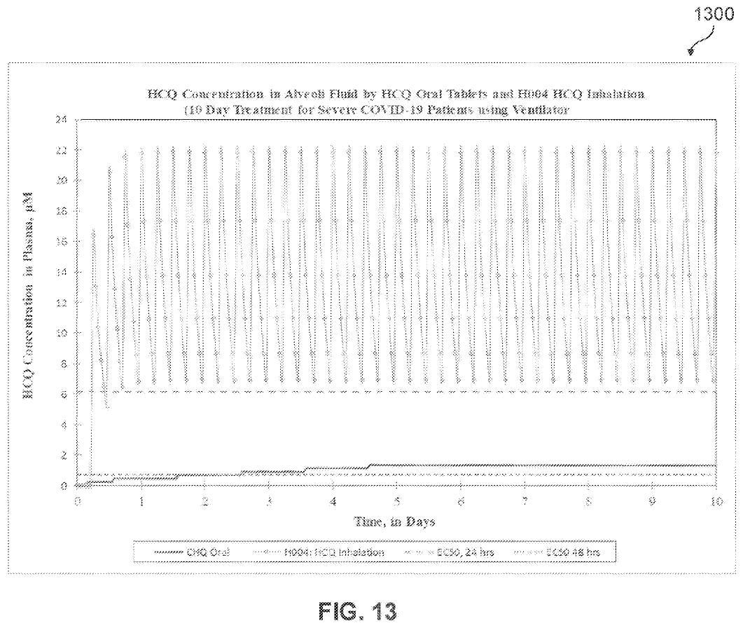

The study indicated that, as a result of taking HCQ oral tablets, the concentration of HCQ in the alveolar fluid (where a significant amount of CoV2 incubates) is estimated to be 0.45 μM at Day-1 (800 mg dose) and 1.3 μM at Day-5 (total dose of 2,400 mg), as demonstrated in plot 100 in FIG. 1. The estimated curve in FIG. 1 for HCQ concentration in human plasma is based on (i) Cmax in plasma of HCQ with 200 mg oral tablet dose, (ii) the corresponding tmax, (iii) HCQ's half-life in human plasma, and (iv) dose used by the treatment for COVID-19 in 5 days. A similar analysis was performed for HCQ administered by inhalation. As shown in plots 1200 and 1300, shown in FIGS. 12 and 13, respectively, the concentration of HCQ in the alveolar fluid was estimated to reach up to 22 μM after administration of HCQ by inhalation.

The known HCQ EC50 for inhibition of CoV2 is 6.14 μM for 24 hrs and 0.72 μM for 48 hrs. However, in first two (2) treatment days, the HCQ concentrations (Day-1 0.23 and 0.45 μM after the 1st and 2nd 400 mg dose in Day-1, respectively, and Day-2 0.67 μM) are below the EC50s. This explains why the low HCQ concentration in alveolar fluid contributed by HCQ oral tablets may be insufficient and therefore likely suboptimal for anti-viral treatment against this respiratory Virus.

The dosing regimen for the off-label use of oral HCQ tablets may not be sufficient to reach the therapeutic threshold for combating COVID-19. However, the dose of oral tablet HCQ cannot be further increased. Clinical experience has shown that higher doses are likely to be excessively toxic. This is one of the reasons why HCQ oral tablet therapy remains controversial and, perhaps, is the reason for its unproven efficacy against CoV2.

### Cascade Impactor Results for Inhalable HCQ

As discussed above, the particle size of an inhalation drug, namely the API, can be measured by a Cascade Impactor (Westech Instruments), which consists of multiple stages (0-7). The particle sizes at each stage are listed in Table 8, which represents the drug delivery to different portions of the entire respiratory tract using a stand-alone MDI actuator, for example actuator 500, discussed above.

TABLE 8
Particle Sizes at Different Stages of Respiratory
Airway Portion Measured by Cascade Impactor

| | Typical Particle | Respiratory |
|---|---|---|
| Stage # | Size Range | Airway Portion |
| 0 | 9.0 μm-10.0 μm | Nasopharynx |
| 1 | 5.8 μm-9.0 μm | |
| 2 | 4.7 μm-5.8 μm | Pharynx |
| 3 | 3.3 μm-4.7 μm | Trachea & Primary Bronchi |
| 4 | 2.1 μm-3.3 μm | Secondary Bronchi |
| 5 | 1.1 μm-2.1 μm | Terminal Bronchi |
| 6 | 0.65 μm-1.1 μm | Alveoli |
| 7 | 0.43 μm-0.65 μm | |

The Cascade Impactor data of the disclosed inventions were analyzed, and these results demonstrate that the drug particle delivery percentage throughout the upper and lower airway tract, as well as in the deep lung portion, such as the alveolus, were as follows: (i) 45% of particles residing on stages 6 and 7 can reach alveoli to combat CoV2 in the Alveoli; and (ii) 51% of particles residing on stages 3 to 5 deliver HCQ from trachea to terminal bronchi in the upper and lower respiratory to fight against CoV2 that may be located there.

After selection of actuator, more formulations with strength from 175 mcg to 850 mcg and with ethanol concentration from 4.5 to 8% w/w were studied by using MDI Actuator C. By comparison both the delivery rate on plate 3-5 and plate 6-filter of these formulations in Tables 9, 10 and 11, as well as the bar charts, it indicated that formulation 5 (200 mcg strength, 5% EtOH) would be efficient for HCQ delivery to the lung and it is selected to be HCQ formulation.

### HCQ Pharmaceutical Formulations for Inhalation Administration

A series of HCQ aerosol formulations were studied, each containing the HCQ free base in a strength ranging from 175 mcg to 850 mcg (i.e., ˜0.38 to 0.75 percent), an ethanol (“EtOH”) concentration ranging from 4% to 12%, and a HFA propellant concentration ranging from 91 to about 96 percent by weight as summarized in Table 9. The Andersen performance of these formulations showed that formulation 5 was a viable choice according to the delivery efficiency of stage 3-5 and stage 6-filter.

TABLE 9
Formulations of Examples 1-5

| Materials | Function | Example 1 | Example 2 | Example 3 | Example 4 | Example 5 |
|---|---|---|---|---|---|---|
| Hydroxychloroquine | API | 0.38% w/w | 0.38% w/w | 0.443% w/w | 0.62% w/w | 1.242% w/w |
| (HCQ) free base | | | | | | |
| Ethanol | Co-solvent | 5% w/w | 5% w/w | 5.5% w/w | 6% w/w | 7% w/w |
| Poloxamer 124 or 182 | Surfactant | — | — | — | Poloxamer 124, | Poloxamer 182, |
| | | | | | 0.02%, w/w | 0.2% w/w |
| HFA 134a | Propellant | 94.62% w/w | 94.62% w/w | 94.057% w/w | 93.36% | HFA 227, |
| | | | | | | 91.76% w/w |

### Example 1 (Formulation 1 in Table 7)

In one embodiment, the formulation contains 0.38% w/w HCQ free base, 5% w/w EtOH, and 94.62% w/w HFA 134a, which was prepared by:
i) adding 0.63 g mixture of HCQ base and EtOH into an aerosol canister and crimping a 50 μL valve onto it. The mixture is HCQ base solution from NaOH method and Anhydrous EtOH (1:1.828 w/w ratio)
ii) Pressurized filling ˜11.07 g HFA 134a into the canister and mixing well.
iii) Cascade Impactor tests showed that FPM (3-filter) 161.5 μg (92.3%) and EPM (6-filter) is 86.8 μg (49.6%) per actuation.

### Example 2 (Formulation 2 in Table 7)

In one embodiment, the formulation contains 0.38% w/w HCQ free base, 5% w/w EtOH, and 94.62% w/w HFA 134a, which was prepared by:
i) adding 44.5 mg HCQ base from EtOAc method and 0.585 g anhydrous ethanol into an aerosol canister and crimping a 50 μL valve onto it.
ii) Pressurized filling ˜11.07 g HFA 134a into the canister and mixing well.
iii) Cascade Impactor tests showed that FPM (3-filter) 168.3 μg (93.2% delivery rate) and EPM (6-filter) is 94.3 μg (53.9% delivery rate) per actuation.

### Example 3 (Formulation 10 in Table 7)

In one embodiment, the formulation contains 0.443% w/w HCQ free base, 5.5% w/w EtOH and 94.057% w/w HFA 134a, which was prepared by:
i) adding 51.8 mg HCQ base from EtOAc method and 0.644 g anhydrous ethanol into an aerosol canister and crimping a 50 μL valve onto it.
ii) Pressurized filling ˜11.0 g HFA 134a into the canister and mixing well.
iii) Cascade Impactor tests showed that FPM (3-filter) 199.1 μg (97.1%) and EPM (6-filter) is 94.3 μg (46.0%) per actuation.

### Example 4 (Formulation 11 in Table 7)

In one embodiment, the formulation contains 0.620% w/w HCQ free base, 5.5% w/w EtOH and 94.057% w/w HFA 134a, which is prepared by:
i) adding 72.5 mg HCQ base from EtOAc method and 0.703 g anhydrous ethanol into an aerosol canister and crimping a 50 μL valve onto it.
ii) Pressurized filling ˜11.0 g HFA 134a into the canister and mixing well.

### Example 5 (Formulation 12 in Table 7)

In one embodiment, the formulation contains 1.242% w/w HCQ free base, 7% w/w EtOH and 91.558% w/w HFA 227, which is prepared by:
iii) adding 145.2 mg HCQ base from EtOAc method and 0.820 g anhydrous ethanol into an aerosol canister and crimping a 50 μL valve onto it.
iv) Pressurized filling ˜11.0 g HFA 227 into the canister and mixing well.

TABLE 10
HCQ Pharmaceutical Formulations 1-12

| Formulation | HCQ-base | EtOH | HFA 134a | Surfactant | API |
|---|---|---|---|---|---|
| No. | % w/w | % w/w | % w/w | % w/w | Strength |
| 1 | 0.380% | 4.000% | 95.620% | — | 175 mcg |
| 2 | 0.380% | 5.000% | 94.620% | — | 175 mcg |
| 3 | 0.540% | 5.000% | 94.460% | — | 250 mcg |
| 4 | 1.080% | 12.000% | 86.920% | — | 500 mcg |
| 5 | 0.430% | 5.000% | 94.570% | — | 200 mcg |
| 6 | 0.600% | 6.000% | 93.400% | — | 275 mcg |
| 7 | 0.760% | 8.000% | 91.240% | — | 350 mcg |
| 8 | 0.443% | 4.500% | 95.057% | — | 205 mcg |
| 9 | 0.443% | 5.000% | 94.557% | — | 205 mcg |
| 10 | 0.443% | 5.500% | 94.057% | — | 205 mcg |
| 11 | 0.620% | 6.000% | 93.360% | Poloxamer | 400 mcg |
| | | | | 124, | |
| | | | | 0.02% | |
| 12 | 1.242% | 7.000% | HFA 227, | Poloxamer | 850 mcg |
| | | | 91.558% | 182, 0.2% | |

In Table 10, above, Formulations 1-12 are exemplary embodiments of the disclosed HCQ pharmaceutical formulations for treating a pulmonary disease, such as COVID-19. In particular, as shown further below, Formulation 5 advantageously provided the most effective results in terms of delivery to a patient's upper respiratory track and a deep portion of the lung where a plurality of alveoli are located.

Initially, several formulations with API strengths from 175 mcg to 500 mcg and with ethanol concentration from 4% to 12% w/w were evaluated by Andersen tests using MDI Actuator A. With higher strength, higher concentration of ethanol is necessary for dissolving the API completely. Results showed that with higher strength of 500 mcg, the delivery rate of stage 3-5 and stage 6-filter is 9% and 10%, respectively, which is quite low. The lower strength of 175 mcg (formulation 2), the delivery rate of stage 3-5 and stage 6-filter is 21% and 22%, respectively, which is better than high API strength.

### Examples of Nozzle Size Selection for MDI Actuators Configured for Stand-Alone Use

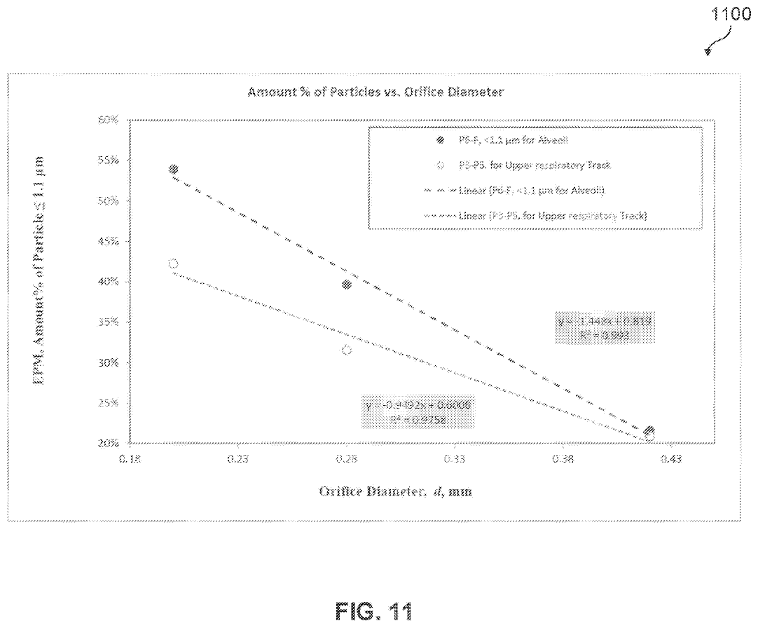

As described above, the disclosed MDI actuator nozzles may include an inner diameter that is optimized to dispense fine API particle sizes, such as API particles having a diameter of about 1.1 μm or less. Table 12, below, outlines a study of the amount percentage (%) of fine API particles having particle diameters of less than about 1.1 μm versus nozzle inner diameter size of the disclosed MDI actuators, for example MDI actuator 500. These results are also outlined in graph 1100 of FIG. 11. The general components of MDI actuator 500 was utilized, but different nozzle inner diameter sizes were tested in this study. In particular, 3 different MDI actuators were tested: (1) MDI actuator having 0.42 mm nozzle inner diameter (“MDI Actuator A”), (2) MDI actuator having 0.28 mm nozzle inner diameter (“MDI Actuator B”), and (3) MDI actuator having 0.20 mm nozzle inner diameter (“MDI Actuator C”). These 3 MDI Actuators (A-C) all had a nozzle with a jet length of about 0.7 mm.

MDI Actuator A, MDI Actuator B, and MDI Actuator C were tested using the same pharmaceutical formulation, in particular an HCQ pharmaceutical formulation having a strength of 0.175 mg (or 175 mcg) of HCQ. The HCQ was HCQ free base and was 0.38% (w/w), 5.0% ethanol alcohol (w/w), 94.62% propellant HFA 134a (w/w) (“w/w” denotes weight by weight).

As shown in Table 12, the Items represent the different Cascade Impactor particle size distribution (in μm) of a respiratory tract, as was described and shown in FIG. 2. As discussed above, the alveoli are primarily located in at least Stage 6, which has a particle diameter of 0.65 μm to 1.1 μm.

“EPM (6-filter)” represents the total amount and delivery efficiency rate, per actuation, of HCQ particles having a diameter of less than about 1.1 μm. The delivery efficiency rate was determined by dividing (i) a total amount, per actuation, of HCQ particles having a diameter of less than about 1.1 μm, by (ii) an expected API metered dose per actuation. In the tests outlined in Table 12, the expected API metered dose per actuation was 175 mcg, and the total amount is the total amount, per actuation, of HCQ particles having a diameter of less than about 1.1 μm.

Table 13 shows that using MDI Actuator C (nozzle 0.20 mm), the delivery rate of stage 3-5 and stage 6-filter is 42% and 54%, respectively, which is much higher than the one with MDI Actuator A (nozzle 0.42 m) and the one with actuator B (0.28 mm). Table 12 and Plot 1100, shown in FIG. 11, demonstrate the relationship between the actuator nozzle size and the delivery rate. It demonstrated that the delivery rate is approximately linear with actuator nozzle size:

TABLE 11
HCQ Actuators

| Actuators | A | B | C |
|---|---|---|---|
| Nozzle Size (mm) | 0.42 | 0.28 | 0.20 |

As shown in Table 13, MDI Actuator C, which had an nozzle inner diameter of about 0.20 mm, provided the strongest results in terms of delivery efficiency rate, as compared to MDI Actuator A or MDI Actuator B. In particular, MDI Actuator C provided a delivery efficiency rate of about 53.9% for “P6-F, <1.1 μm for Alveoli,” meaning that about 53.9% of the HCQ particles dispensed, per actuation, by MDI Actuator C had particle diameters of less than 1.1 μm. As discussed above, this particle size is advantageous in delivering HCQ to a portion of the lungs in Stage 6, and is therefore effective in treating pulmonary diseases, such as COVID-19, within the alveoli. Therefore, with respect to “P6-F, <1.1 μm for Alveoli,” MDI Actuator C, with a delivery efficiency rate of 53.9% represents a significant improvement of MDI Actuators A-B having delivery rates of 21.6% and 39.7%, respectively. Accordingly, MDI Actuator C was selected for HCQ.

TABLE 12
Formulation 2 with MDI Actuator A, MDI Actuator B, and MDI Actuator C

| | | Formulation 2, | | Formulation 2, | | Formulation 3, | |
|---|---|---|---|---|---|---|---|
| | | Actuator A | | Actuator B | | Actuator C | |
| | | 175 mcg | | 175 mcg | | 175 mcg | |
| | | | Compared | | Compared | | |
| Items | | | to | | to | | Compared |
| Stage # | Spec | 1 test | strength | 1 test | strength | 1 test | to strength |
| Ada/Thr | | 82.3 | 47.0% | 24.3 | 13.9% | 11.8 | 6.8% |
| 0 | >9.0 | 1.4 | 0.8% | 0.6 | 0.3% | 0.4 | 0.2% |
| 1 | p1 5.8-9 | 0.8 | 0.4% | 0.6 | 0.3% | 0.5 | 0.3% |
| 2 | p2 4.7-5.8 | 1.5 | 0.8% | 1.3 | 0.7% | 0.8 | 0.5% |
| 3 | p3 3.3-4.7 | 8.6 | 4.9% | 7.7 | 4.4% | 6.5 | 3.7% |
| 4 | p4 2.1-3.3 | 13.7 | 7.8% | 19.4 | 11.1% | 25.7 | 14.7% |
| 5 | p5 1.1-2.1 | 14.2 | 8.1% | 28.2 | 16.1% | 41.7 | 23.9% |
| 6 | p6 0.65-1.1 | 4.4 | 2.5% | 13.4 | 7.7% | 23.2 | 13.3% |
| 7 | p7 0.43- | 2.0 | 1.2% | 4.7 | 2.7% | 6.5 | 3.7% |
| | 0.65 | | | | | | |
| Filter | <0.43 | 31.5 | 18.0% | 51.3 | 29.3% | 64.6 | 36.9% |

| | | | | | | |
|---|---|---|---|---|---|---|
| Total | 160.3 | 91.6% | 151.6 | 86.6% | 181.9 | 103.9% |
| % Recovery | 92% | 0.5% | 87% | 0.5% | 104% | 0.6% |
| Total 3~5 | 36.5 | 20.9% | 55.4 | 31.6% | 74.0 | 42.3% |
| CPM (Adaptor/Throat-2) | 86.0 | 49.1% | 26.8 | 15.3% | 13.6 | 7.8% |
| EPM (6-filter) | 37.9 | 21.6% | 69.4 | 39.7% | 94.3 | 53.9% |
| ISM (0-filter) | 78.0 | 44.6% | 127.2 | 72.7% | 170.1 | 97.2% |
| FPM (3-filter) | 74.4 | 42.5% | 124.8 | 71.3% | 168.3 | 96.2% |

TABLE 13
Assessment of Amount of Small Particles
That Can Reach Alveoli for HCQ MDI

| Actuators | A | B | C |
|---|---|---|---|
| Nozzle Size (mm) | 0.42 | 0.28 | 0.20 |
| Amount % of Particle | | | |
| P6-F, <1.1 μm for Alveoli | 21.6% | 39.7% | 53.9% |
| P3-P5, for Upper respiratory Track | 20.9% | 31.6% | 42.3% |

TABLE 14
Different Formulations with MDI Actuator A

| | | Formulation 1, | | Formulation 2, | | Formulation 3, | | Formulation 4, | |
|---|---|---|---|---|---|---|---|---|---|
| | | Actuator A | | Actuator A | | Actuator A | | Actuator A | |
| | | 175 mcg | | 175 mcg | | 250 mcg | | 500 mcg | |
| | | | Compared | | Compared | | Compared | | Compared |
| Items | | | to | | to | | to | | to |
| Stage # | Spec | 1 test | strength | 1 test | strength | 1 test | strength | 1 test | strength |
| Ada/Thr | | 84.1 | 48.1% | 82.3 | 47.0% | 126.2 | 50.5% | 339.1 | 67.8% |
| 0 | >9.0 | 0.9 | 0.5% | 1.4 | 0.8% | 2.3 | 0.9% | 10.1 | 2.0% |
| 1 | pl 5.8-9 | 0.6 | 0.4% | 0.8 | 0.4% | 1.5 | 0.6% | 2.9 | 0.6% |
| 2 | p2 4.7-5.8 | 1.1 | 0.7% | 1.5 | 0.8% | 3.0 | 1.2% | 6.4 | 1.3% |
| 3 | p3 3.3-4.7 | 7.9 | 4.5% | 8.6 | 4.9% | 15.3 | 6.1% | 18.2 | 3.6% |
| 4 | p4 2.1-3.3 | 14.7 | 8.4% | 13.7 | 7.8% | 19.4 | 7.8% | 16.2 | 3.2% |
| 5 | p5 1.1-2.1 | 17.5 | 10.0% | 14.2 | 8.1% | 17.7 | 7.1% | 10.6 | 2.1% |
| 6 | p6 0.65-1.1 | 5.6 | 3.2% | 4.4 | 2.5% | 7.0 | 2.8% | 2.9 | 0.6% |
| 7 | p7 0.43-0.65 | 2.1 | 1.2% | 2.0 | 1.2% | 2.9 | 1.1% | 2.4 | 0.5% |
| Filter | <0.43 | 28.5 | 16.3% | 31.5 | 18.0% | 30.6 | 12.2% | 43.8 | 8.8% |

| | | | | | | | | |
|---|---|---|---|---|---|---|---|---|
| Total | 163.1 | 93.2% | 160.3 | 91.6% | 225.8 | 90.3% | 452.7 | 90.5% |
| % Recovery | 93% | NA | 92% | NA | 90% | NA | 91% | NA |
| Total 3~5 | 40.1 | 22.9% | 36.5 | 20.9% | 52.3 | 20.9% | 45.0 | 9.0% |
| CPM (Adaptor/Throat-2) | 86.8 | 49.6% | 86.0 | 49.1% | 133.0 | 53.2% | 358.5 | 71.7% |
| EPM (6-filter) | 36.2 | 20.7% | 37.9 | 21.6% | 40.5 | 16.2% | 49.1 | 9.8% |
| ISM (0-filter) | 78.9 | 45.1% | 78.0 | 44.6% | 99.6 | 39.8% | 113.5 | 22.7% |
| FPM (3-filter) | 76.3 | 43.6% | 74.4 | 42.5% | 92.8 | 37.1% | 94.1 | 18.8% |

TABLE 15
Different Formulations with Actuator C

| | | Formulation 2, | | Formulation 5, | | Formulation 6, | | Formulation 7, | | Formulation 8, | |
|---|---|---|---|---|---|---|---|---|---|---|---|
| | | Actuator C | | Actuator C | | Actuator C | | Actuator C | | Actuator C | |
| | | 175 mcg | | 200 mcg | | 275 mcg | | 350 mcg | | 205 mcg | |
| | | | Compare | | Compare | | Compare | | Compare | | Compare |
| Items | | | to | | to | | to | | to | | to |
| Stage # | Spec | 1 test | strength | 1 test | strength | 1 test | strength | 1 test | strength | 1 test | strength |
| Ada/ | | 11.8 | 6.8% | 11.1 | 5.6% | 20 | 7.3% | 39.8 | 11.4% | 14.8 | 7.2% |
| Thr | | | | | | | | | | | |
| 0 | >9.0 | 0.4 | 0.2% | 0.5 | 0.2% | 0.8 | 0.3% | 1.3 | 0.4% | 0.8 | 0.4% |
| 1 | p1 | 0.5 | 0.3% | 1.7 | 0.3% | 1 | 0.4% | 1.7 | 0.5% | 1.2 | 0.6% |
| | 5.8-9 | | | | | | | | | | |
| 2 | p2 | 0.8 | 0.5% | 1.5 | 0.7% | 2.5 | 0.9% | 3.8 | 1.1% | 2.1 | 1.0% |
| | 4.7-5.8 | | | | | | | | | | |
| 3 | p3 | 6.5 | 3.7% | 8.1 | 4.0% | 14.7 | 5.4% | 26.1 | 7.5% | 12.3 | 6.0% |
| | 3.3-4.7 | | | | | | | | | | |
| 4 | p4 | 25.7 | 14.7% | 30.1 | 15.0% | 53.3 | 19.4% | 72.9 | 20.8% | 42.6 | 20.8% |
| | 2.1-3.3 | | | | | | | | | | |
| 5 | p5 | 41.7 | 23.9% | 59.9 | 30.0% | 74.8 | 27.2% | 89.4 | 25.5% | 64.4 | 31.4% |
| | 1.1-2.1 | | | | | | | | | | |
| 6 | p6 | 23.2 | 13.3% | 24.6 | 12.3% | 29.1 | 10.6% | 29.2 | 8.3% | 26.9 | 13.1% |
| | 0.65-1.1 | | | | | | | | | | |
| 7 | p7 | 6.5 | 3.7% | 7.8 | 3.9% | 8.2 | 3.0% | 8.8 | 2.5% | 8.2 | 4.0% |
| | 0.43-0.65 | | | | | | | | | | |
| Filter | <0.43 | 64.6 | 36.9% | 67.1 | 33.5% | 56.3 | 20.5% | 89.7 | 25.6% | 65 | 31.7% |
| Total % | | 181.9 | 103.9% | 211.3 | 105.6% | 260.8 | 94.8% | 362.7 | 103.6% | 238.1 | 116.2% |
| Recovery | | 104% | NA | 106% | NA | 95% | NA | 104% | NA | 116% | NA |
| Plate 3~5 | | 74 | 42.3% | 98.1 | 49.0% | 142.8 | 51.9% | 188.4 | 53.8% | 119.3 | 58.2% |
| CPM | | 13.6 | 7.8% | 13.8 | 6.9% | 24.3 | 8.8% | 46.6 | 13.3% | 18.8 | 9.2% |
| (Adaptor/Throat-2) | | | | | | | | | | | |
| EPM (6-filter) | | 94.3 | 53.9% | 99.4 | 4 9.7% | 93.7 | 34.1% | 128 | 36.5% | 100 | 48.8% |
| ISM (0-filter) | | 170.1 | 97.2% | 200.1 | 100.1% | 240.8 | 87.6% | 322.9 | 92.3% | 223.4 | 109.0% |
| FPM (3-filter) | | 168.3 | 96.2% | 197.5 | 98.7% | 236.5 | 86.0% | 316.1 | 90.3% | 219.4 | 107.0% |
| | | | | Formulation 9, | | Formulation 10, | | Formulation 11, | | Formulation 12, | |
| | | | | Actuator C | | Actuator C | | Actuator C | | Actuator C | |
| | | | | 205 mcg | | 205 mcg | | 400 mcg | | 850 mcg | |
| | | | | | Compare | | Compare | | Compare | | Compare |
| | | Items | | | to | | to | | to | | to |
| | | Stage # | Spec | 1 test | strength | 1 test | strength | 1 test | strength | 1 test | strength |
| | | Ada/Thr | | 15.4 | 7.5% | 17.7 | 8.6% | 55.6 | 13.9% | 158.9 | 18.7% |
| | | 0 | >9.0 | 0.6 | 0.3% | 0.6 | 0.3% | 1.3 | 0.3% | 16.6 | 2.0% |
| | | 1 | p1 | 0.6 | 0.3% | 1.2 | 0.6% | 1.6 | 0.4% | 30.5 | 3.6% |
| | | | 5.8-9 | | | | | | | | |
| | | 2 | p2 | 1.6 | 0.8% | 1.6 | 0.8% | 4.5 | 1.1% | 60.9 | 7.2% |
| | | | 4.7-5.8 | | | | | | | | |
| | | 3 | p3 | 9.6 | 4.7% | 9.5 | 4.6% | 29.5 | 7.4% | 181.0 | 21.3% |
| | | | 3.3-4.7 | | | | | | | | |
| | | 4 | p4 | 33.8 | 16.5% | 36.3 | 17.7% | 72.6 | 18.1% | 182.0 | 21.4% |
| | | | 2.1-3.3 | | | | | | | | |
| | | 5 | p5 | 64.8 | 31.6% | 59 | 28.8% | 103.1 | 25.8% | 126.9 | 14.9% |
| | | | 1.1-2.1 | | | | | | | | |
| | | 6 | p6 | 24.9 | 12.2% | 21.7 | 10.6% | 40.2 | 10.0% | 35.0 | 4.1% |
| | | | 0.65-1.1 | | | | | | | | |
| | | 7 | p7 | 7.8 | 3.8% | 7.3 | 3.6% | 11.7 | 2.9% | 14.8 | 1.7% |
| | | | 0.43-0.65 | | | | | | | | |
| | | Filter | <0.43 | 65.4 | 31.9% | 65.3 | 31.8% | 83.3 | 20.8% | 85.5 | 10.1% |

| | | | | | | | | |
|---|---|---|---|---|---|---|---|---|
| Total % | 224.6 | 109.5% | 220.2 | 107.4% | 403.3 | 100.8% | 892.2 | 105.0% |
| Recovery | 110% | NA | 107% | NA | 101% | NA | 105.0% | NA |
| Plate 3~5 | 108.2 | 52.8% | 104.8 | 51.1% | 205.1 | 51.3% | 489.9 | 57.6% |
| CPM | 18.2 | 8.9% | 21.1 | 10.3% | 62.9 | 15.7% | 266.9 | 31.4% |
| (Adaptor/Throat-2) | | | | | | | | |
| EPM (6-filter) | 98.2 | 47.9% | 94.3 | 46.0% | 135.3 | 33.8% | 135.3 | 15.9% |
| ISM (0-filter) | 209.2 | 102.0% | 202.5 | 98.8% | 347.8 | 86.9% | 733.3 | 86.3% |
| FPM (3-filter) | 206.4 | 100.7% | 199.1 | 97.1% | 340.4 | 85.1% | 625.2 | 73.6% |

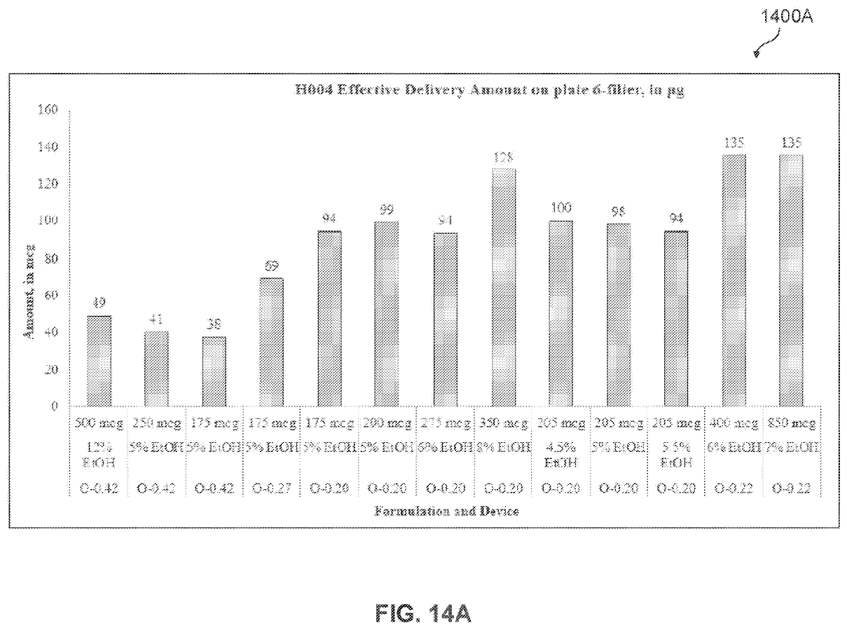

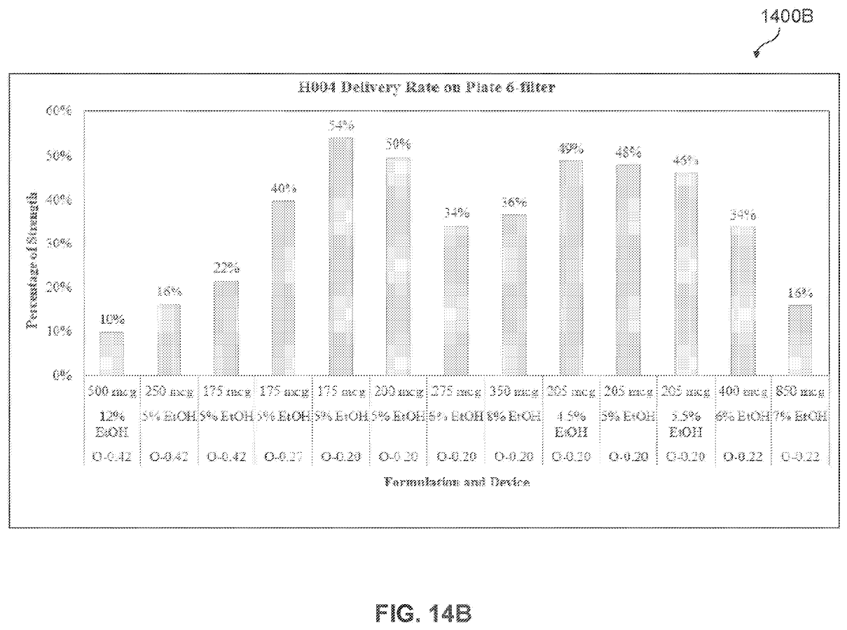

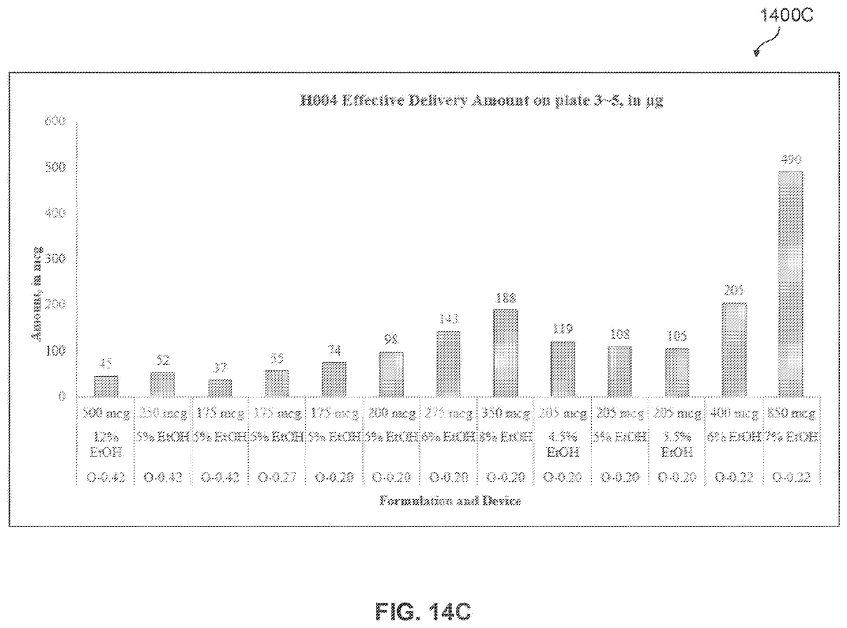

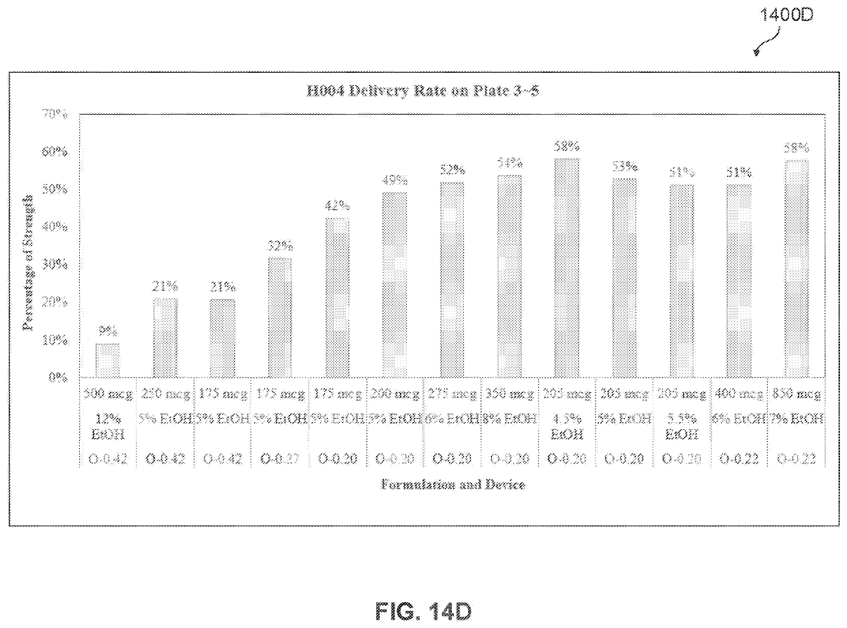

Tables 16 and 17, as well as bar charts 1400A-1400D, shown in FIGS. 14A-14D, respectively, show HCQ delivery amount and delivery rate on plate 6-filter and plate 3-5, respectively, in different formulations (API strength from 175 mcg to 500 mcg and ethanol concentration from 4.5% to 12% w/w) and different actuators configured for stand-alone use (nozzle having an inner diameter from 0.20 mm to 0.42 mm). The results shown in Table 16, below, evidence that formulation 5 with MDI Actuator C was a viable choice for HCQ.

TABLE 16
HCQ Delivery Efficiency for Deep Lung (Alveoli) (Plate 6-filter)

| | | | | | Amount of | Deep Lung |
|---|---|---|---|---|---|---|
| | | | | | API | (Alveoli) |
| | | | | API | Delivered | API |
| Formulation | Formulation and Device | | | Strength | <1.1 mcm | Delivery |
| No. | | | API Strength | Per 1 | Per 1 | Rate Per 1 |
| (From | | | Per 1 Metered | Metered | Metered | Metered |
| Table 1) | Actuator | EtOH | Dose | Dose | Dose | Dose |
| 4 | O-0.42 | 12% EtOH | 500 mcg | 500 | 49.1 | 9.8% |
| 3 | O-0.42 | 5% EtOH | 250 mcg | 250 | 40.5 | 16.2% |
| 2 | O-0.42 | 5% EtOH | 175 mcg | 175 | 37.9 | 21.7% |
| 2 | O-0.27 | 5% EtOH | 175 mcg | 175 | 69.4 | 39.7% |
| 2 | O-0.20 | 5% EtOH | 175 mcg | 175 | 94.3 | 53.9% |
| 5 | O-0.20 | 5% EtOH | 200 mcg | 200 | 99.4 | 49.7% |
| 6 | O-0.20 | 6% EtOH | 275 mcg | 275 | 93.7 | 34.1% |
| 7 | O-0.20 | 8% EtOH | 350 mcg | 350 | 127.7 | 36.5% |
| 8 | O-0.20 | 4.5% EtOH | 205 mcg | 205 | 100 | 48.8% |
| 9 | O-0.20 | 5% EtOH | 205 mcg | 205 | 98.2 | 47.9% |
| 10 | O-0.20 | 5.5% EtOH | 205 mcg | 205 | 94.3 | 46.0% |
| 11 | O-0.22 | 6% EtOH | 400 mcg | 400 | 135.3 | 33.8% |
| 12 | O-0.22 | 7% EtOH | 850 mcg | 850 | 135.3 | 15.9% |

TABLE 17
HCQA Delivery Efficiency for Upper Respiratory Tract (Plate 3-5)

| | | | | | | Upper |
|---|---|---|---|---|---|---|
| | Formulation and Device | | | | | Respiratory |
| | | | API | API | Amount API | Tract API |
| Formulation | | | Strength | Strength | Delivered | Delivery |
| No. | | | Per 1 | Per 1 | <5 mcm Per | Rate Per 1 |
| (From | | | Metered | Metered | 1 Metered | Metered |
| Table 1) | Actuator | EtOH | Dose | Dose | Dose | Dose |
| 4 | O-0.42 | 12% EtOH | 500 mcg | 500 | 45 | 9.0% |
| 3 | O-0.42 | 5% EtOH | 250 mcg | 250 | 52.3 | 20.9% |
| 2 | O-0.42 | 5% EtOH | 175 mcg | 175 | 36.5 | 20.9% |
| 2 | O-0.27 | 5% EtOH | 175 mcg | 175 | 55.4 | 31.7% |
| 2 | O-0.20 | 5% EtOH | 175 mcg | 175 | 74 | 42.3% |
| 5 | O-0.20 | 5% EtOH | 200 mcg | 200 | 98.1 | 49.1% |
| 6 | O-0.20 | 6% EtOH | 275 mcg | 275 | 142.8 | 51.9% |
| 7 | O-0.20 | 8% ELOH | 350 mcg | 350 | 188.4 | 53.8% |
| 8 | O-0.20 | 4.5% EtOH | 205 mcg | 205 | 119.3 | 58.2% |
| 9 | O-0.20 | 5% EtOH | 205 mcg | 205 | 108.2 | 52.8% |
| 10 | O-0.20 | 5.5% EtOH | 205 mcg | 205 | 104.8 | 51.1% |
| 11 | O-0.22 | 6% EtOH | 400 mcg | 400 | 205.1 | 51.3% |
| 12 | O-0.22 | 7% EtOH | 850 mcg | 850 | 489.9 | 57.6% |

### Examples of Delivery Efficiencies for Inhalable HCQ Delivered Via MDI Actuators Configured for Use with an Auxiliary Delivery Component

Described below are examples of Andersen evaluation results for MDI actuators which may be configured for use with an auxiliary delivery component, for example a ventilator.

### Example 1

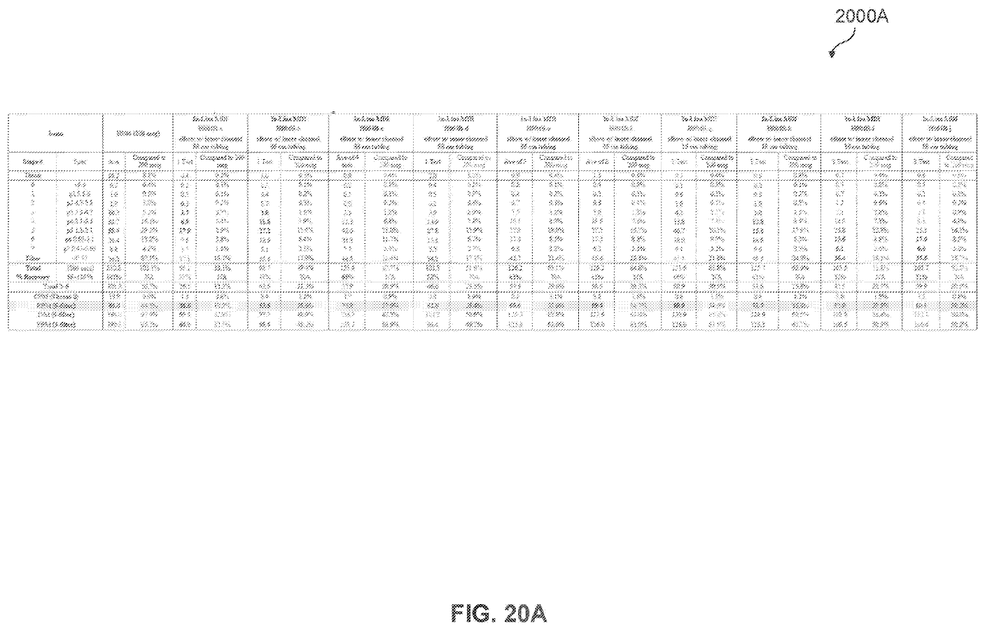

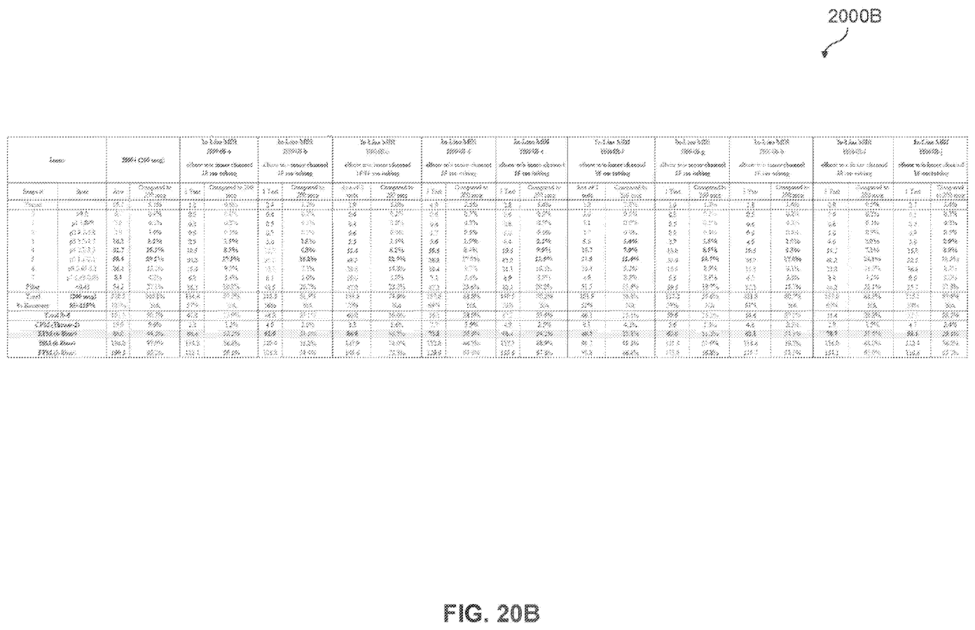

Actuator H004B-a was applied for HCQ in-line Andersen evaluation. Prime HCQ valve by discharging a predetermined number of actuations to waste. Discharge 10 actuations with actuator H004B-a into the cascade impaction sampling apparatus through an elbow connection w/inner channel and an in-line tubing (55 cm long). The air flow rate for the Andersen test is set to 28.3 L/min. As shown in table 2000A, shown in FIG. 20A, with actuator H004B-a, FPM of HCQ was 64.9 μg (delivery efficiency rate is 32.5%) and EPM was 38.5 μg (delivery efficiency rate is 19.2%).

### Example 2

Actuator H004B-c was applied for HCQ in-line Andersen evaluation. Prime HCQ valve by discharging a predetermined number of actuations to waste. Discharge 10 actuations with actuator H004B-c into the cascade impaction sampling apparatus through an elbow connection with an inner channel and an in-line tubing (55 cm long). As shown in table 2000A, With actuator H004B-c, FPM of HCQ was 133.7 μg (66.9%) and EPM was 75.8 μg (37.9%).

### Example 3

Actuator H004B-i was applied for HCQ in-line Andersen evaluation. Prime HCQ valve by discharging a predetermined number of actuations to waste. Discharge 10 actuations with actuator H004B-i into the cascade impaction sampling apparatus through an elbow connection without an inner channel and an in-line tubing (15 cm long). As shown in table 2000B, shown in FIG. 20B, with actuator H004B-i, FPM of HCQ was 134.1 μg (67.0%) and EPM was 75.7 μg (37.8%).

Tables 19 and 20, below, as well as bar charts 1500A-1500F, shown in FIGS. 15A-15F, respectively, show HCQ delivery amount and delivery rate for various MDI actuators configured to connect to auxiliary delivery components, for example ventilators, via an elbow connection.

TABLE 18
HCQ Formulation (200 mcg HCQ base from actuator)

| | HCQ-base | EtOH | HFA | Total | HCQ-base | EtOH % | HFA % |
|---|---|---|---|---|---|---|---|
| Formulation | (g) | (g) | (g) | Weight-g | % w/w | w/w | w/w |
| Each | 0.0503 | 0.5850 | 11.065 | 11.7 | 0.4299% | 5.0000% | 94.5701% |
| Canister | | | | | | | |

TABLE 19
HCQB Delivery Efficiency for Alveoli (Elbow Connection with Inner Channel)

| | Formulation and Device | | | | | Delivered | Delivery |
|---|---|---|---|---|---|---|---|
| # | API | Actuator | EtOH | Strength | Strength | <1.1 mcm | Rate |
| 1 | HCQ Base | O-0.20 | 5% EtOH | 200 mcg | 200 | 89 | 44.5% |
| 2 | HCQ Base | in-Line Tube (55 cm), HCQB-a, | 5% EtOH | 200 mcg | 200 | 38.5 | 19.3% |
| | | connection w/inner channel | | | | | |
| 3 | HCQ Base | in-Line Tube (55 cm), HCQB-b, | 5% EtOH | 200 mcg | 200 | 53.8 | 26.9% |
| | | connection w/inner channel | | | | | |
| 4 | HCQ Base | in-Line Tube (55 cm), HCQB-c, | 5% EtOH | 200 mcg | 200 | 75.8 | 37.9% |
| | | connection w/inner channel, Ave | | | | | |
| 5 | HCQ Base | in-Line Tube (55 cm), HCQB-d, | 5% EtOH | 200 mcg | 200 | 52.8 | 26.4% |
| | | connection w/inner channel | | | | | |
| 6 | HICQ Base | in-Line Tube (55 cm), HCQB-e, | 5% EtOH | 200 mcg | 200 | 66.0 | 33.0% |
| | | connection w/inner channel, Ave | | | | | |
| 7 | HCQ Base | in-Line Tube (55 cm), HCQB-f, | 5% EtOH | 200 mcg | 200 | 69.4 | 34.7% |
| | | connection w/inner channel, Ave | | | | | |
| 8 | HCQ Base | in-Line Tube (15 cm), HCQB-g, | 5% EtOH | 200 mcg | 200 | 68.0 | 34.0% |
| | | connection w/inner channel | | | | | |
| 9 | HCQ Base | in-Line Tube (15 cm), HCQB-h, | 5% EtOH | 200 mcg | 200 | 71.7 | 35.9% |
| | | connection w/ inner channel | | | | | |
| 10 | HCQ Base | in-Line Tube (15 cm), HCQB-i, | 5% EtOH | 200 mcg | 200 | 55.0 | 27.5% |
| | | connection w/inner channel | | | | | |
| 11 | HCQ Base | in-Line Tube (55 cm), HCQB-j, | 5% EtOH | 200 mcg | 200 | 60.5 | 30.3% |
| | | connection w/ inner channel | | | | | |

TABLE 20
HCQB Delivery Efficiency on Plate 3~5 (Elbow Connection w/ Inner Channel)

| | Formulation and Device | | | | | Delivered | Delivery |
|---|---|---|---|---|---|---|---|
| # | API | Actuator | EtOH | Strength | Strength | <4.7 mcm | Rate |
| 1 | HCQ Base | O-0.20 | 5% EtOH | 200 mcg | 200 | 101.3 | 50.7% |
| 2 | HCQ Base | in-Line Tube (55 cm), HCQB-a, | 5% EtOH | 200 mcg | 200 | 26.5 | 13.3% |
| | | connection w/inner channel | | | | | |
| 3 | HCQ Base | in-Line Tube (55 cm), HCQB-b, | 5% EtOH | 200 mcg | 200 | 42.6 | 21.3% |
| | | connection w/inner channel | | | | | |
| 4 | HCQ Base | in-Line Tube (55 cm), HCQB-c, | 5% EtOH | 200 mcg | 200 | 57.9 | 29.0% |
| | | connection w/inner channel | | | | | |
| 5 | HCQ Base | in-Line Tube (55 cm), HCQB-d, | 5% EtOH | 200 mcg | 200 | 46.6 | 23.3% |
| | | connection w/inner channel | | | | | |
| 6 | HCQ Base | in-Line Tube (55 cm), HCQB-e, | 5% EtOH | 200 mcg | 200 | 57.9 | 29.0% |
| | | connection w/inner channel | | | | | |
| 7 | HCQ Base | in-Line Tube (55 cm), HCQB-f, | 5% EtOH | 200 mcg | 200 | 56.5 | 28.3% |
| | | connection w/inner channel | | | | | |
| 8 | HCQ Base | in-Line Tube (15 cm), HCQB-g, | 5% EtOH | 200 mcg | 200 | 60.9 | 30.5% |
| | | connection w/inner channel | | | | | |
| 9 | HCQ Base | in-Line Tube (15 cm), HCQB-h, | 5% EtOH | 200 mcg | 200 | 51.6 | 25.8% |
| | | connection w/inner channel | | | | | |
| 10 | HCQ Base | in-Line Tube (15 cm), HCQB-i, | 5% EtOH | 200 mcg | 200 | 45.5 | 22.8% |
| | | connection w/inner channel | | | | | |
| 11 | HCQ Base | in-Line Tube (55 cm), HCQB-j, | 5% EtOH | 200 mcg | 200 | 39.9 | 20.0% |
| | | connection w/inner channel | | | | | |

TABLE 21
HCQB Delivery Efficiency for Alveoli (Elbow Connection w/o Inner Channel)

| | Formulation and Device | | | | | Delivered | Delivery |
|---|---|---|---|---|---|---|---|
| # | API | Actuator | EtOH | Strength | Strength | <1.1 mcm | Rate |
| 1 | HCQ Base | O-0.20 | 5% EtOH | 200 mcg | 200 | 89 | 44.5% |
| 2 | HCQ Base | in-Line Tube (15 cm), HCQB-a, | 5% EtOH | 200 mcg | 200 | 64.4 | 32.2% |
| | | connection w/o inner channel | | | | | |
| 3 | HCQ Base | in-Line Tube (15 cm), HCQB-b, | 5% EtOH | 200 mcg | 200 | 62.6 | 31.3% |
| | | connection w/o inner channel | | | | | |
| 4 | HCQ Base | in-Line Tube (15 cm), HCQB-c, | 5% EtOH | 200 mcg | 200 | 86.6 | 43.3% |
| | | connection w/o inner channel | | | | | |
| 5 | HCQ Base | in-Line Tube (15 cm), HCQB-d, | 5% EtOH | 200 mcg | 200 | 73.8 | 36.9% |
| | | connection w/o inner channel, Ave | | | | | |
| 6 | HCQ Base | in-Linc Tube (15 cm), HCQB-c, | 5% EtOH | 200 mcg | 200 | 68.4 | 34.2% |
| | | connection w/o inner channel | | | | | |
| 7 | HCQ Base | in-Line Tube (15 cm), HCQB-f, | 5% EtOH | 200 mcg | 200 | 46.3 | 23.2% |
| | | connection w/o inner channel | | | | | |
| 8 | HCQ Base | in-Line Tube (15 cm), HCQB-g, | 5% EtOH | 200 mcg | 200 | 63.0 | 31.5% |
| | | connection w/o inner channel | | | | | |
| 9 | HCQ Base | in-Line Tube (15 cm), HCQB-h, | 5% EtOH | 200 mcg | 200 | 62.1 | 31.1% |
| | | connection w/o inner channel | | | | | |
| 10 | HCQ Base | in-Line Tube (15 cm), HCQB-i, | 5% EtOH | 200 mcg | 200 | 75.7 | 37.9% |
| | | connection w/o inner channel | | | | | |
| 11 | HCQ Base | in-Line Tube (15 cm), HCQB-j, | 5% EtOH | 200 mcg | 200 | 58.1 | 29.1% |
| | | connection w/o inner channel | | | | | |

### Examples of In Vivo Testing of Deep Lung Delivery of Inhaled HCQ

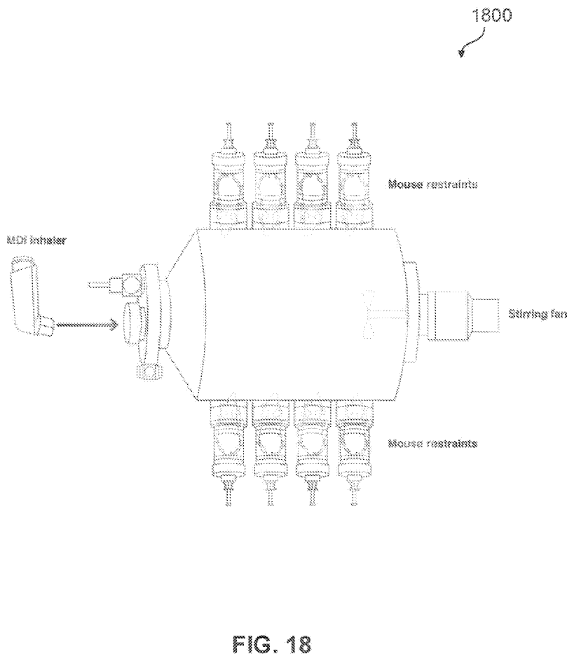

As a proof of concept, an in vivo study was designed to determine if HCQ could be detected in the lungs to demonstrate effective delivery. Mice can breathe the aerosol of drug products. A breathing tank is used for mice to breathe the aerosol of the drug product, such as HCQ. The drug product is administered through the specially designed stainless steel breathing tank 1800, for example as shown in FIG. 18.

The exposure tank size is designed such that the total breathing volume of all eight mice during a 10-minute breathing treatment (1.8 L) is less than 10% of the tank size (21.5 L). The internal wall of the tank is electrically polished to minimize its adsorption of the study drug. Eight mice were mounted to the tank with four mice on each side using small animal restraints. At the start of each treatment session, an effective amount of the drug was administered into pre-cleaned tank. A stirring fan installed inside the tank was set to promote circulation of the pharmaceutical agent. Specifically, the fan was set at 400 RPM in this study and turned on before the pharmaceutical agent was administered. Thirty seconds after the last spray (t=0 minute), eight mice were mounted to the inhalation chamber to breathe the air from inside the breathing tank for 10 minutes, and then were taken off the breathing tank. Samples from the mice were taken, starting immediately after removal from the tank, to perform pharmacokinetic studies.

Pharmacokinetic studies performed after the mice were removed from the tank showed that 28% of HCQ was adsorbed by the wall of the breathing tank. The net HCQ concentration in the tank chamber was calculated to be 58.6 μg/L. The representative tidal volume for mice is 22.5 mL/min with 150 breaths per minute. It was calculated that each mouse breathed 13.2 μg of HCQ. Based on the body weight ratio, this H004 dose corresponds to 12.2 times of the relative dose for humans.

The lungs of the mice were collected and homogenated at eight (8) time points of 10 minutes, 30 minutes, 45 minutes, 1 hour, 2 hour, 3 hour, 4 hour, and 6 hour after cessation of the breathing treatment. In total 32 mice were studied for each time point. The HCQ in the lungs was analyzed using an LC/MS/MS method.

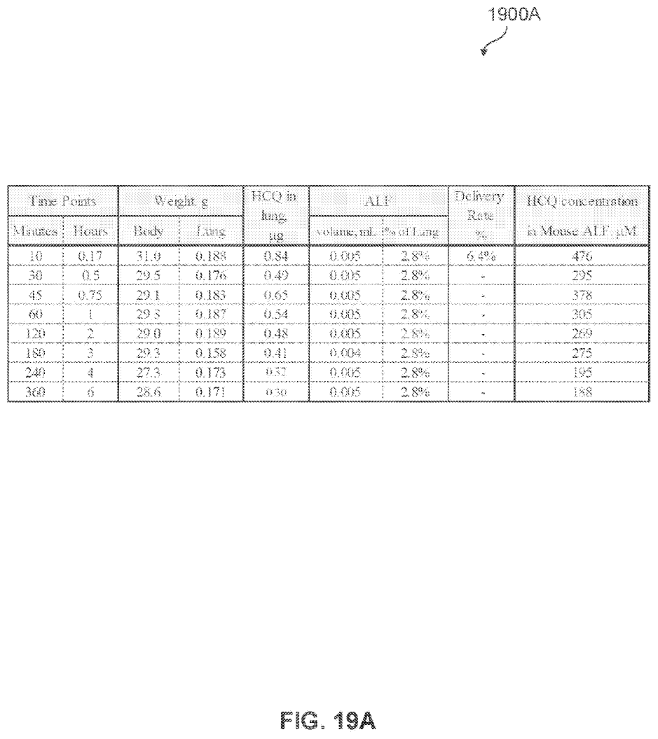

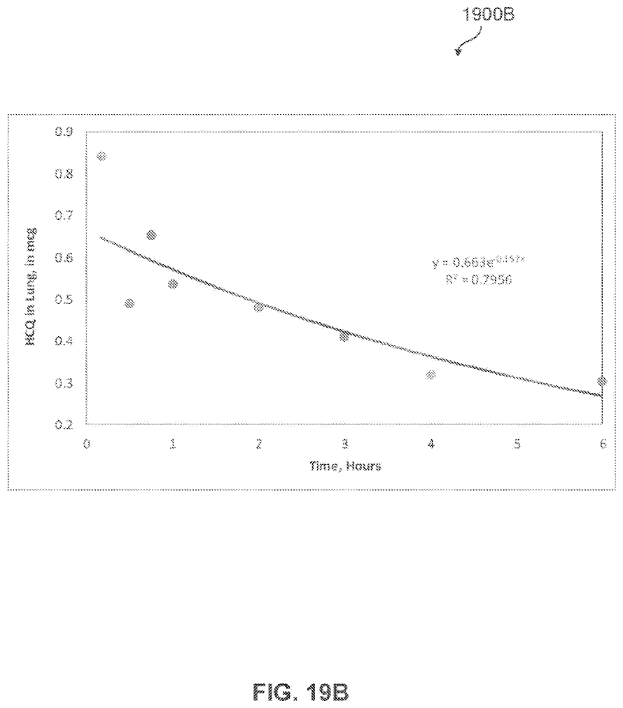

The results from this study are summarized in Table 1900A and Plot 1900B, shown in FIGS. 19A and 19B, respectively. Plot 1900B provides the relation of the HCQ amount in the mouse lungs and time. The study results demonstrated that all 32 mice appeared healthy, with no signs of distress during and after a high dose of the HCQ treatment.

The mouse ALF volumes shown in Table 1900A were estimated based on the typical human ALF volume (36 mL), and the ratio of mouse lung weight to human lung weight (1.3 kg). Because all HCQ quantities in the mouse lung tissues were diffused from ALF, the HCQ concentration in the ALF right after the treatment could be estimated per the HCQ amount in the lung tissues.

While various embodiments have been described herein, they have been presented by way of example, and not limitation. It should be apparent that adaptations and modifications are intended to be within the meaning and range of equivalents of the disclosed embodiments, based on the teaching and guidance presented herein. It therefore will be apparent to one skilled in the art that various changes in form and detail can be made to the embodiments disclosed herein without departing from the spirit and scope of the present disclosure. The elements of the embodiments presented herein are not necessarily mutually exclusive, but may be interchanged to meet various situations as would be appreciated by one of skill in the art.

Embodiments of the present disclosure are described in detail herein with reference to embodiments thereof as illustrated in the accompanying drawings, in which like reference numerals are used to indicate identical or functionally similar elements. References to “one embodiment,” “an embodiment,” “some embodiments,” “in certain embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The examples are illustrative, but not limiting, of the present disclosure. Other suitable modifications and adaptations of the variety of conditions and parameters normally encountered in the field, and which would be apparent to those skilled in the art, are within the spirit and scope of the disclosure.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage or mode of operation.

Unless otherwise defined herein, scientific and technical terms used in connection with embodiments of present disclosure shall have the meanings that are commonly understood by those of ordinary skill in the art. Nomenclatures used in connection with, and techniques described herein are those known and commonly used in the art. Also, descriptions of well-known functions and constructions are omitted for clarity and conciseness.

As used herein, the singular forms “a,” “an,” and “the” are intended to include the plural forms as well, unless the context clearly indicates otherwise. It will be further understood that the terms “comprises,” “comprising,” “includes,” “including,” “have” and/or “having” when used herein, specify the presence of stated features, integers, steps, operations, elements, and/or components, but do not preclude the presence or addition of one or more other features, integers, steps, operations, elements, components, and/or groups thereof.

Where a range of numerical values is recited herein, comprising upper and lower values, unless otherwise explicitly stated, the range is intended to include the endpoints thereof, and all integers and fractions within the range. It is not intended that the scope of the claims be limited to the specific values recited when defining a range. Further, when an amount, concentration, or other value or parameter is given as a range, this is to be understood as specifically disclosing all ranges formed from any pair of any upper range limit and any lower range limit, regardless of whether such pairs are separately disclosed. Finally, when the term “about” is used in describing a value or an end-point of a range, the disclosure should be understood to include the specific value or end-point referred to. Whether or not a numerical value or end-point of a range recites “about,” the numerical value or end-point of a range is intended to include two embodiments: one modified by “about,” and one not modified

As used herein, the terms “about” or “approximately” means plus or minus 10% of the stated numerical value. For example, about 5% means 4.5% to 5.5%.

As used herein, the terms “treating” or “treatment” refer to reducing severity, eliminating, or a combination thereof, with respect to a particular disease, condition, or injury. Thus, in the context of the disclosed methods of treatment of COVID-19, the disclosed methods are intended to: (i) reduce severity, (ii) eliminate, or (iii) reduce severity and eliminate COVID-19. As described, common symptoms of COVID-19 include dry cough, difficulty breathing (e.g. shortness of breath), fever (e.g. body temperature of 100.4° Fahrenheit or more), fatigue, and others. Thus, the disclosed methods for treating COVID-19 may reduce and/or eliminate some of these symptoms of COVID-19 over a specified period of time.

The present embodiment(s) have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

It is to be understood that the phraseology or terminology used herein is for the purpose of description and not of limitation. The breadth and scope of the present disclosure should not be limited by any of the above-described exemplary embodiments, but should be defined in accordance with the following claims and their equivalents.

What is claimed:

1. A metered-dose inhaler (“MDI”) actuator for self-administration of pharmaceutical formulations comprising: a handheld MDI actuator for dispensing, via actuation, a pharmaceutical formulation from a MDI and into a patient, the pharmaceutical formulation having at least one active pharmaceutical ingredient (API); wherein the MDI is capable of administering a portion of the at least one API to a portion of a lung where a plurality of alveoli are located; and the MDI actuator comprises: an nozzle having an inner diameter of 0.15 mm to 0.3 mm.

2. The MDI actuator of claim 1, wherein the inner diameter of the nozzle is about 0.18-0.25 mm.

3. The MDI actuator of claim 1, wherein the inner diameter of the nozzle is about 0.20-0.23 mm.

4. The MDI actuator of claim 1, wherein the portion of the lung where the plurality of alveoli are located includes at least Stage 6 based on a Cascade Impactor particle size distribution of a respiratory tract, wherein Stage 6 has a particle diameter size of about 1.1 μm or less.

5. The MDI actuator of claim 1, wherein the MDI actuator is capable of providing a delivery efficiency rate of at least 25.0%, wherein the delivery efficiency rate is determined by dividing (i) a total amount, per actuation, of an API having a particle diameter of less than 1.1 μm, by (ii) an expected API metered dose per actuation.

6. The MDI actuator of claim 1, wherein the MDI actuator is capable of providing a delivery efficiency rate of at least 25.0%, wherein the delivery efficiency rate is determined by dividing (i) a total amount, per actuation, of an API having a particle diameter of less than 1.1 mih, by (ii) an expected API metered dose per actuation, wherein the API is hydroxychloroquine (HCQ), and the API dose strength per actuation is 400 μg.

7. The MDI actuator of claim 1, wherein the pharmaceutical formulation comprises a pharmaceutical formulation suitable for inhalation.

8. The MDI actuator of claim 1, wherein the pharmaceutical formulation comprises a pharmaceutical formulation suitable for inhalation, and further comprises an API comprising an anti-viral therapeutic agent, wherein the anti-viral therapeutic agent comprises HCQ, a free base thereof, or a pharmaceutically acceptable salt thereof.

9. The MDI actuator of claim 1, wherein the pharmaceutical formulation is indicated for the treatment of a pulmonary disease.

10. The MDI actuator of claim 1, wherein the pharmaceutical formulation is indicated for the treatment or prophylaxis of COVID-19.

11. The MDI actuator of claim 1, wherein the patient has one or more pulmonary diseases.

12. The MDI actuator of claim 1, wherein the patient has one or more pulmonary diseases, including at least COVID-19.

13. The MDI actuator of claim 1, wherein the container is a pressurized canister for dispensing, per actuation, a metered dose of the pharmaceutical formulation.

14. The MDI actuator of claim 1, wherein nozzle further comprises a jet length of 0.5 mm to 1.0 mm.

15. The MDI actuator of claim 1, wherein nozzle further comprises a jet length of about 0.7 mm.

16. The MDI actuator of claim 8, wherein the pharmaceutical formulation further comprises: an alcohol of about 5% (w/w) of the pharmaceutical formulation; a propellant of about 95% (w/w) of the pharmaceutical formulation; and wherein “w/w” denotes weight by weight.

17. The MDI actuator of claim 8, wherein the pharmaceutical formulation further comprises: an alcohol of about 5% (w/w) of the pharmaceutical formulation, wherein the alcohol is ethanol alcohol (“EtOH”); a propellant of about 94.6% (w/w) of the pharmaceutical formulation, wherein the propellant is HFA-134a; the HCQ is about 0.4% (w/w) of the pharmaceutical formulation; the HCQ is free base, the pharmaceutical formulation is a true solution; the pharmaceutical formulation has a total weight of about 8-12.5 grams; wherein “w/w” denotes weight by weight.

18. The MDI actuator of claim 1, wherein the pharmaceutical formulation comprises an inhalable steroid.

19. The MDI actuator of claim 18, wherein the inhalable steroid is selected from the group consisting of flunisolide, fluticasone furoate, fluticasone propionate, triamcinolone acetonide, beclomethasone dipropionate, budesonide, mometasone furoate, ciclesonide, and pharmaceutically acceptable salts thereof.

20. The MDI actuator of claim 1, wherein the pharmaceutical formulation comprises a bronchodilator.

21.-213. (canceled)
